(19) United States
(12) Patent Application Publication
(10) Pub. No.: US 2021/0330678 A1
(43) Pub. Date: Oct. 28, 2021

(54) PAPD5 INHIBITORS AND METHODS OF USE THEREOF

(71) Applicant: Children`s Medical Center Corporation, Boston, MA (US)

(72) Inventors: Suneet Agarwal, Lexington, MA (US); Neha Nagpal, Boston, MA (US)

(21) Appl. No.: 17/273,937

(22) PCT Filed: Sep. 5, 2019
(86) PCT No.: PCT/US2019/049819

§ 371 Date: Mar. 5, 2021

Related U.S. Application Data
(60) Provisional application No. 62/819,147, filed on Mar. 15, 2019.
(60) Provisional application No. 62/727,443, filed on Sep. 5, 2018.

(51) Int. Cl. A61K 31/553; A61K 31/4745; A61K 31/4025; A61K 31/5025; A61K 31/5365; A61K 31/4375; A61P 7/06; C12N 5/074; C12N 5/0783; C12N 5/0735

(52) U.S. Cl. CPC A61K 31/553; A61K 31/4745; A61K 31/4025; A61K 31/5025; A61K 31/5365; C12N 2501/999; A61P 7/06; C12N 5/0696; C12N 5/0636; C12N 5/0606; C12N 5/0646; A61K 31/4375

(57) ABSTRACT

The disclosure relates to compounds that are, e.g., PAP Associated Domain Containing 5 (PAPD5) inhibitors and methods of use thereof.

### CLAIM OF PRIORITY

This application claims priority U.S. Provisional Patent Application Ser. No. 62/727,443, filed on Sep. 5, 2018, and U.S. Provisional Patent Application Ser. No. 62/819,147, filed on Mar. 15, 2019, the entire contents of which are hereby incorporated by reference.

### STATEMENT AS TO FEDERALLY SPONSORED RESEARCH

This invention was made with government support under Grant No. DK107716, awarded by the National Institutes of Health. The government has certain rights in the invention.

### TECHNICAL FIELD

The present disclosure relates to compounds that inhibit PAP Associated Domain Containing 5 (PAPD5), and to methods of using these compounds to treat conditions such as telomere diseases, and aging-related and other degenerative disorders.

### BACKGROUND

A telomere is a region of repetitive nucleotide sequences at each end of a chromosome, which protects the end of the chromosome from deterioration or from fusion with neighboring chromosomes. The length of a telomere is a key determinant of cellular self-renewal capacity. The telomerase ribonucleoprotein maintains telomere length in tissue stem cells, and its function is critical for human health and longevity.

Short telomeres, due to genetic or acquired insults, cause a loss of cellular self-renewal and result in life-threatening diseases, for which there are few if any effective medical therapies. In these diseases involving short telomeres, e.g., aplastic anemia, pulmonary fibrosis, hepatic cirrhosis, bone marrow failure, etc., there is an unmet clinical need for new therapies.

### SUMMARY

Poly(A) ribonuclease (PARN) mutations can result in the accumulation of 3′ oligo-adenylated forms of nascent Telomerase RNA Component (TERC) RNA transcripts, which are targeted for destruction, thus causing telomerase deficiency and telomere diseases. Disruption of the non-canonical poly(A) polymerase PAP Associated Domain Containing 5 (PAPD5; also known as Topoisomerase-related function protein 4-2 (TRF4-2)) may restore TERC levels, telomerase activity, and telomere elongation in PARN-mutant patient cells.

In one general aspect, the disclosure relates to a method of treating a disease or condition selected from:
disorder associated with telomere or telomerase dysfunction; and/or
a disorder associated with aging; and/or
a pre-leukemic or pre-cancerous condition; and/or
neurodevelopmental disorder,
   the method comprising administering to a subject in need thereof a therapeutically effective amount of a compound of Formula (I):

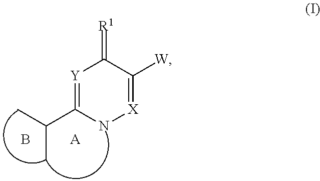

or a pharmaceutically acceptable salt thereof, or a pharmaceutical composition comprising same.

In another general aspect, the disclosure relates to a method of treating a disease or condition selected from:
disorder associated with telomere or telomerase dysfunction; and/or
a disorder associated with aging; and/or
a pre-leukemic or pre-cancerous condition; and/or
neurodevelopmental disorder,
   the method comprising administering to a subject in need thereof a therapeutically effective amount of a compound of Formula (II):

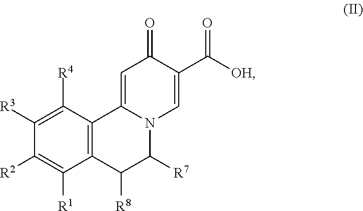

or a pharmaceutically acceptable salt thereof, or a pharmaceutical composition comprising same.

In yet another general aspect, the disclosure provides a method of modulating ex vivo expansion of stem cells, the method comprising contacting the cells with an effective amount of a compound of Formula (I) or Formula (II), or a pharmaceutically acceptable salt thereof.

In yet another general aspect, the disclosure provides a method of modulating non-coding RNAs in a cell, the method comprising contacting the cell with an effective amount of a compound of Formula (I) or Formula (II), or a pharmaceutically acceptable salt thereof.

In yet another general aspect, the disclosure provides a method of expanding a cell, the method comprising culturing the cell in the presence of an effective amount of a compound f Formula (I) or Formula (II), or a pharmaceutically acceptable salt thereof.

In some embodiments, the present application provides a compound of Formula (I), or a pharmaceutically acceptable salt thereof. In some embodiments, the present application provides a compound of Formula (II), or a pharmaceutically acceptable salt thereof.

In some embodiments, the present application provides a composition comprising a compound of Formula (I), or a pharmaceutically acceptable salt thereof. In some embodiments, the present application provides a composition comprising a compound of Formula (II), or a pharmaceutically acceptable salt thereof.

In some embodiments, the present application provides a use of a compound of Formula (I), or a pharmaceutically acceptable salt thereof, or a composition comprising same, in the manufacture of a medicament for the treatment of any one of the disease or conditions described herein.

In some embodiments, the present application provides a use of a compound of Formula (II), or a pharmaceutically acceptable salt thereof, or a composition comprising same in the manufacture of a medicament for the treatment of any one of the disease or conditions described herein.

In some embodiments, the present application provides a use of a compound of Formula (I), or a pharmaceutically acceptable salt thereof, or a composition comprising same, for the treatment of any one of the disease or conditions described herein.

In some embodiments, the present application provides a use of a compound of Formula (II), or a pharmaceutically acceptable salt thereof, or a composition comprising same for the treatment of any one of the disease or conditions described herein.

In some embodiments, the present application provides a compound of Formula (I), or a pharmaceutically acceptable salt thereof, or a composition comprising same for use in the treatment of any one of the disease or conditions described herein.

In some embodiments, the present application provides a compound of Formula (II), or a pharmaceutically acceptable salt thereof, or a composition comprising same for use in the treatment of any one of the disease or conditions described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 is a schematic diagram showing an exemplary model for TERC 3′ end maturation by PARN.

FIG. 2 is a schematic diagram showing an exemplary model of reciprocal regulation of TERC maturation by PARN and PAPD5.

FIG. 3A is a schematic diagram showing PAPD5 can polyadenylate RNA oligonucleotides in vitro.

FIG. 3B shows PAPD5 has a strong preference for ATP when PAPD5 polyadenylates RNA oligonucleotides.

FIG. 4A is a schematic diagram showing an assay for determining that a compound is a PAPD5 inhibitor.

FIG. 4B is a graph showing luminescence signal generated in a high throughput screening setting for reactions performed using no enzyme, wildtype PAPD5, and mutant PAPD5 at different input ATP concentrations.

FIG. 5 shows the results of PAPD5 oligonucleotide adenylation assay with wildtype PAPD5, and mutant PAPD5.

FIG. 6 shows activity of DHQ-1 ((S)-6-isopropyl-10-methoxy-9-(3-methoxypropoxy)-2-oxo-6,7-dihydro-2H-pyrido[2,1-a]isoquinoline-3-carboxylic acid) in inhibiting rPAPD5-mediated RNA oligonucleotide extension in vitro.

FIG. 7 shows that DHQ (DHQ-1) and inhibitor 1 restore telomere length in DC patient iPS cells

FIG. 8 shows activity of DHQ-1 ((S)-6-isopropyl-10-methoxy-9-(3-methoxypropoxy)-2-oxo-6,7-dihydro-2H-pyrido[2,1-a]isoquinoline-3-carboxylic acid) in restoring telomerase RNA (TERC) 3′ end processing and TERC RNA steady state levels.

FIG. 9 shows rPAPD5 inhibition in vitro by inhibitor 2, inhibitor 1, and DHQ (DHQ-1).

FIG. 10 shows that inhibitor 2 does inhibit PARN exonuclease whereas inhibitor 1 and DHQ (DHQ-1) do not inhibit PARN.

FIG. 11 shows that compounds DHQ (DHQ-1) and inhibitor 1 do not inhibit multiple poly-nucleotide polymerases.

FIG. 12 shows that inhibitor 1 and DHQ (DHQ-1) restore telomerase RNA (TERC) end processing whereas inhibitor 2 does not.

FIG. 13 shows activity of compounds DHQ-1, 18C and 19C in RNA oligo-adenylation assay.

FIG. 14 shows activity of compounds DHQ-1, 20C and 1C in RNA oligo-adenylation assay.

FIG. 15 shows activity of compounds DHQ-1, 3C and 22C in RNA oligo-adenylation assay.

FIG. 16 shows activity of compounds DHQ-1, 2C, 7C-1, 7C-2, and 12C in RNA oligo-adenylation assay.

FIG. 17 shows activity of compounds DHQ-1, 4C, 5C, 9C, and 10C in RNA oligo-adenylation assay.

FIG. 18. DHQ-1 binds and inhibits rPAPD5, and restores TERC and telomere length in DC patient iPSCs. (a) Inhibition of recombinant PAPD5 (rPAPD5) by DHQ-1 in the low micromolar range. (b) Inhibitory activity of DHQ-1 (100 μM) across a panel of canonical and non-canonical poly-nucleotide polymerases (yeast poly(A) polymerase (PAP), E. coli PAP, rPAPD4, and S. pombe Cid1). (c) Direct binding of DHQ-1 to rPAPD5. Dose-dependent melting temperature shifts as revealed by differential scanning fluorimetry. ATP*=3′-azidomethyl-ATP, non-extendable ATP analog. (d) TERC RNA levels by Northern blot in normal (WT) and PARN-mutant iPSCs treated with DHQ-1 or DMSO for 2 weeks. 18S rRNA is shown as a loading control. (e) TERC 3′ end profiles by RLM-RACE in normal (WT) and PARN-mutant DC patient iPSCs, untreated or treated with DHQ-1 at indicated concentrations versus DMSO for 1 week. (f) Quantitation of cumulative oligo-adenylation of TERC species in PARN-mutant DC patient iPSCs treated with DHQ-1 versus DMSO, by deep sequencing of TERC 3′ RACE amplicons. (g) Southern blot of telomere length by TRF analysis in normal (WT) versus PARN-mutant patient iPSCs cells, untreated or treated with DHQ-1 at various concentrations versus DMSO for 3 weeks.

FIG. 19 PAPD5 inhibitors augment TERC and telomere length in PARN-deficient primary human HSPCs in vitro and in vivo. (a) DHQ-1. (b) Inference of CRISPR edits (ICE) analysis 5 days after CRISPR/Cas9 ribonucleoprotein transfection of human CD34+ cells targeting PARN, showing ˜95% indel formation. Similar results were achieved targeting PAPD5, TERC, and AAVS1 loci. (c) TERC 3¢ end profiles in primary human HSPCs following CRISPR targeting of AAVS1 versus PARN, followed by 5 days in vitro culture with DMSO or DHQ-1. (d) Quantitation of oligo-adenylated TERC by deep sequencing amplicons shown in (c). (e) TERC RNA levels in human HSPCs following inactivation of AAVS1 versus PARN, followed by 5 days in vitro culture with DMSO or RG7834 (1 μM). (f) TERC 3¢ end profiles in primary PARN-mutant patient CD34+ cells following 5 days in vitro culture with small molecules, with quantitation by deep sequencing (right). (g) TERC 3¢ end profiles of human CD34+ cells recovered and sorted from whole bone marrow, 6 weeks after xenotransplantation of CRISPR-engineered human HSPCs into NBSGW mice, treated with DMSO versus DHQ-1 in drinking water, with quantitation by deep sequencing (right). Each data point reflects pooling of human CD34+ cells from 4 mice in each category. (h) TERC 3¢ end profiles in human CD19+ cells recovered 6 weeks after xenotransplantation as in (g). Data points reflect cells from three individual mice in each category. Quantitation by deep sequencing (below) depicts mean and standard error (n=5 mice per category). (i) Flow-FISH telomere length measurement in human CD45+ cells from whole bone marrow, 6 weeks after xenotransplantation (n=3 mice per category). Statistics: one-way ANOVA, Tukey's multiple comparison test (ns: not significant, *P<0.05, **P<0.01, ***P<0.0005).

FIG. 20 Oral bioavailability of DHQ-1 and impact on human HSPC engraftment and differentiation in xenotransplantation. (a) Random plasma concentration of DHQ-1 when DHQ-1 (125 μM) versus DMSO is administered in drinking water to mice xenotransplanted with PARN-targeted human HSPCs (n=5). (b) Deep sequencing profiles of TERC RNA 3′ ends in human CD19+ cells recovered from xenotransplanted mice, as in FIG. 2h. (c) Flow-FISH analysis of total bone marrow cells recovered from xenotransplanted mice. (Upper) Human cells (hCD45+) cells are readily distinguished from mouse cells (hCD45−) due to long telomere length in mouse cells. (Lower) Flow-FISH-based telomere length fluorescence intensity distribution in hCD45+ cells recovered from xenotransplants with AAVS1 versus PARN-targeted HSPCs, treated with DMSO versus RG7834, as in FIG. 2i. One representative trace out of three in each category shown. (d) Human hematopoietic cell engraftment (hCD45+) as a percentage of total mouse plus human CD45+ cells in bone marrow, 6 weeks after xenotransplantation of AAVS1 or PARN-targeted HSPCs, after treatment with DHQ-1 versus DMSO.(n=5 mice per group; ns: not significant). (e) Comparison of human HSPC (CD34+), B-cell (CD19+), and myeloid cell (CD33+) compartments as a percentage of engrafted human CD45+ cells, 6 weeks after xenotransplantation of AAVS1 or PARN-targeted HSPCs, after treatment with DHQ-1 versus DMSO. (n=5 mice per group; ns: not significant).

FIG. 21 shows that DHQ-1 and compounds 18C, 19C restore telomerase RNA (TERC) end processing

FIG. 22 shows that DHQ-1 and compounds 1C, 2C, 3C, 7C-1, 7C-2, and 12C restore telomerase RNA (TERC) end processing.

FIG. 23 shows that DHQ-1 and compounds 4C, 5C, 22C, 9C, and 10C restore telomerase RNA (TERC) end processing.

FIG. 24 shows that compound DHQ-1 and compounds 18C, 19C, 1C, 3C, and 22C elongate telomeres.

FIG. 25 shows that DHQ-1 and compounds 4C, 5C, 22C, 9C, and 10C restore telomerase RNA (TERC) end processing.

FIG. 26 shows that DHQ-1 and compounds 18C, 19C, 1C, 2C, 3C, 4C, 5C, 22C, 12C, 7C-1, 7C-2, 9C, and 10C restore telomerase RNA (TERC) levels

### DETAILED DESCRIPTION

A telomere is a region of repetitive nucleotide sequences at each end of a chromosome. For vertebrates, the sequence of nucleotides in telomeres is TTAGGG. In humans, this sequence of TTAGGG is repeated approximately hundreds to thousands of times. Telomerase is a ribonucleoprotein that adds the telomere repeat sequence to the 3′ end of telomeres. Cells with impaired telomerase function often have limited capacity for self-renewal, i.e., an abnormal state or condition characterized by an inability of cells (e.g., stem cells) to divide sufficiently. This deficiency in cells can, for example, lead to various diseases and disorders.

Telomerase RNA component (TERC) serves at least two functions: (1) it encodes the template sequence used by telomerase reverse transcriptase (TERT) for the addition of hexanucleotide repeats to telomeres, and (2) it is the scaffold that nucleates multiple proteins that target telomerase to the Cajal body, where telomeres are extended.

The disclosure provides compounds and methods to modulate TERC levels, e.g., by using compounds that target TERC, or compounds that modulate the level or activity of PAP Associated Domain Containing 5 (PAPD5) and/or Poly(A) specific ribonuclease (PARN), both of which are involved in the 3′-end maturation of TERC.

Also provided are methods of diagnosing patients and methods of treating patients having various telomere diseases. Various implementations of these compounds and methods are described herein.

Definitions

As used herein, the term “about” means “approximately” (e.g., plus or minus approximately 10% of the indicated value).

The term “telomere disease,” “telomere syndrome,” “disorder associated with telomere dysfunction,” or “disorder associated with telomerase dysfunction” refers to a disorder associated with abnormal telomeres or abnormal telomerase function. They include, but not are limited to, dyskeratosis congenita (DC), Revesz syndrome, Hoyeraal-Hreidarrson syndrome, Coats plus syndrome, and some forms of inherited aplastic anemia, myelodysplastic syndrome, aplastic anemia, pulmonary fibrosis, idiopathic pulmonary fibrosis, bone marrow failure, hematological disorder, hepatic disease (e.g., hepatic fibrosis, chronic liver disease, non-alcoholic steatohepatitis, and hepatic cirrhosis), among others. Telomere diseases also include those affecting the blood and immune systems, lungs, liver, skin, mucosal surfaces, bones, cardiovascular system, endocrine system, and/or gastrointestinal system, as cells with the impaired self-renewal capacity can affect the normal function of organs or systems. Some of these disorders include aplastic anemia, pulmonary fibrosis, hepatic cirrhosis, osteoporosis and osteonecrosis, vascular malformations, diabetes, primary immunodeficiency, and inflammatory bowel disease. This group of diseases is often associated with a cellular state marked with decreased self-renewal capacity that can be attributed to an alteration in telomere length. Telomere disease also includes tissue failure and organ failure. The tissue failure that relates to telomere disease can have various causes, e.g., infection, inflammation, environmental (radiation, chemical, physical insults) causes, medications and chemotherapy, among others. These various causes can all contribute to telomere deficiency.

The term “telomere deficiency” as used herein refers to a cellular state in the body, including stem cells, induced pluripotent cells and fibroblasts, and is often marked by a perturbation in expression or activity of an enzyme that is involved in regulating telomere size. As used herein, the term “telomerase dysfunction” refers to abnormal levels or fabrication of telomerase in a cell or patient. For example, telomerase dysfunction can include telomerase deficiency, such as where telomerase levels are lower than normal due to excess or unwanted telomerase degradation, and telomerase over-activity, such as where telomerase levels are higher than normal due to deficient telomerase degradation.

The terms “subject” and “patient” are used interchangeably throughout the specification and describe an animal, human or non-human. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

The terms “inhibition”, “inhibiting”, “inhibit,” or “inhibitor” refer to the ability of a compound to reduce, slow, halt, and/or prevent activity of a particular biological process in a cell relative to vehicle. In some embodiments, “inhibit”, “block”, “suppress” or “prevent” means that the activity being inhibited, blocked, suppressed, or prevented is reduced by at least 5%, 10%, 15%, 20%, 25%, 30%, 35%, 40%, 45%, 50%, 55%, 60%, 65%, 70%, 75%, 80%, 85%, 90%, 95%, or 100% as compared to the activity of a control (e.g., activity in the absence of the inhibitor).

An “effective amount” refers to an amount sufficient to elicit the desired biological response, i.e., treating cancer. As will be appreciated by those of ordinary skill in this art, the effective amount of the compounds described herein can vary depending on such factors as the desired biological endpoint, the pharmacokinetics of the compound, the condition being treated, the mode of administration, and the age and health of the subject, and the guidance of the treating physician. An effective amount includes that amount necessary to slow, reduce, inhibit, ameliorate or reverse one or more symptoms associated with cancer. For example, in the treatment of cancer, such terms can refer to a reduction in the size of the tumor.

The term “Cₙ₋ₘ alkyl” includes straight-chain alkyl groups (e.g., methyl, ethyl, propyl, butyl, pentyl, hexyl, heptyl, octyl, nonyl, decyl, etc.) and branched-chain alkyl groups (e.g., isopropyl, tert-butyl, isobutyl, etc.). In certain embodiments, a straight chain or branched chain alkyl has twelve or fewer carbon atoms in its backbone (e.g., C₁₋₁₂ for straight chain; C₃₋₁₂ for branched chain). For example, the term C₁₋₁₂ includes alkyl groups containing 1 to 12 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenylene” refers to a divalent alkenyl linking group.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. The term “Cₙ₋ₘ alkynylene” refers to a divalent alkynyl linking group.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each have, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms. As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbamyl” to a group of formula C(O)NH2.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, the term “oxo” refers to an oxygen atom as a divalent substituent, forming a carbonyl group when attached to a carbon (e.g., C═O), or attached to a heteroatom forming a sulfoxide or sulfone group.

As used herein, the term “thioxo” refers to a sulfur atom as a divalent substituent, forming, e.g., a group of formula C═S when attached to a carbon atom, or forming a thiosulfoxide or thiosulfone group, when attached to a heteroatom.

As used herein, the term “thio” refers to a group of formula SH.

As used herein, the term “cyano” refers to a group of formula CN.

As used herein, the term “amino” refers to a group of formula NH₂.

As used herein, the term “carboxy” or “carboxyl” refers to a —C(O)OH group.

As used herein, “halo” or “halogen” refers to F, Cl, Br, or I. In some embodiments, halo is F, Cl, or Br. In some embodiments, halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

The term “n-membered” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the non-aromatic cyclic hydrocarbon, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, or 12 ring-forming atoms. In some embodiments, the cycloalkyl is a 3-12 membered monocyclic or bicyclic cycloalkyl. In some embodiments, the cycloalkyl is a C₃₋₇ monocyclic cycloalkyl. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cyclooctyl, cyclooctenyl, and the like. In some embodiments, cycloalkyl is cyclopropyl, cyclobutyl, cyclopentyl, cyclohexyl, cyclooctyl, or cyclooctenyl. In some embodiments, the cycloalkyl is a cyclooctenyl ring fused with 1 or 2 benzene rings. In some embodiments, the cycloalkyl is a 3-8 membered or 3-7 membered monocyclic cycloalkyl group (e.g., C₃₋₈ or C₃₋₇ cycloalkyl). In some embodiments, the cycloalkyl is a 8-12-membered bicyclic cycloalkyl. In some embodiments, the cycloalkyl is a 8-16-membered bicyclic or tricyclic cycloalkyl (e.g., C₈₋₁₆ cycloalkyl). The term “cycloalkylene” refers to a divalent cycloalkyl linking group.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 membered monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered heteroaryls are pyridyl, pyrazinyl, pyrimidinyl, triazinyl and pyridazinyl. The term “heteroarylene” refers to a divalent heteroaryl linking group.

The term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl and the like. In some embodiments, aryl groups have from 6 to about 20 carbon atoms, from 6 to about 15 carbon atoms, or from 6 to about 10 carbon atoms. In some embodiments, the aryl group is phenyl. The term “arylene” refers to a divalent aryl linking group.

As used herein, “heterocycloalkyl” or “aliphatic heterocycle” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the non-aromatic heterocycle, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. In some embodiments, the heterocycloalkyl is a 8-12-membered heterocycloalkyl (e.g., bicyclic heterocycloalkyl). In some embodiments, the heterocycloalkyl is a 8-16-membered heterocycloalkyl (e.g., bicyclic or tricyclic heterocycloalkyl). In some embodiments, the 8-12 membered bicyclic heterocycloalkyl is a 8-12 membered fused heterocycloalkylaryl group or a 8-12 membered fused heterocycloalkylheteroaryl group. In some embodiments, the heterocycloalkyl is a 9-12 membered bicyclic heterocycloalkyl. In some embodiments, the 9-10 membered bicyclic heterocycloalkyl is a 9-10 membered fused heterocycloalkylaryl group or a 9-10 membered fused heterocycloalkylheteroaryl group. The term “heterocycloalkylene” refers to a divalent heterocycloalkyl linking group.

The term “aromatic” refers to a carbocycle or heterocycle having one or more polyunsaturated rings having aromatic character (i.e., having (4n+2) delocalized π (pi) electrons where n is an integer).

The term “aliphatic” refers to organic compounds (including polymers) in which carbon atoms and heteroatoms form open chains and which do not contain polyunsaturated rings having aromatic character. Aliphatic compounds may be linear or cyclic, saturated or unsaturated, straight chain or branched.

Therapeutic Compounds

Compounds of Formula (I)

In some embodiments, the compound of the present disclosure has Formula

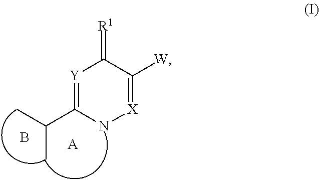

or a pharmaceutically acceptable salt thereof, wherein:

R¹, X, Y, W, ring A, and ring B are as described herein.

Certain embodiments of the Formula (I) are described below.

In some embodiments:

R¹ is selected from O, S, N—OH, N—C₁₋₃ alkoxy, N—NH₂, and N—CN;

W is selected from C(O)OR^{a1}, C(O)NR^{c1}R^{d1}, C(O)NR^{c1}S(O)₂R^{b1}, C(O)NR^{c1}OR^{a1}, NR^{c1}C(O)OR^{a1}, NR^{c1}C(O)NR^{c1}R^{d1}, NR^{c1}C(O)R^{b1}, OR^{a1}, NR^{c1}R^{d1}, NR^{c1}S(O)₂R^{b1}, B(OH)₂, P(═O)(OR^{a1})₂, halo, CN, Cy, and a carboxylic acid bioisostere;

or R¹ and W together with the carbon atoms to which they are attached from a monocyclic 4-7 membered heterocycloalkyl ring or a monocyclic 5-6 membered heteroaryl ring, each of which is optionally substituted with 1, 2, or 3 substituents independently selected from R^{Cy};

X is selected from N and CR²;

Y is selected from N and CR³;

R² is selected from H, Cy, halo, CN, NO₂, OR^{a1}, C₁₋₆alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

R³ is selected from H, Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)²NR^{c1}R^{d1};

ring A, together with N and other atom or atoms that ring A shares with ring B, is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{A};

ring B, together with Y and other atom or atoms that ring B shares with ring A, is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{B};

each R^{A} is independently selected from H, Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

or any two R^{A} groups together with the atom or atoms to which they are attached form ring C, which is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{C};

each R^{B} is independently selected from H, Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

or any two R^{B} groups together with the atom or atoms to which they are attached form ring D, which is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{D};

or any two R^{A} and R^{B} groups together with the atoms to which they are attached form a ring selected from a monocyclic C₃₋₇ cycloalkyl ring, a monocyclic 4-7 membered heterocycloalkyl ring, and a monocyclic 5-6 membered heteroaryl ring, each of which is optionally substituted with 1, 2, 3, 4, or 5 substituents independently selected from R^{Cy};

each R^{C} and R^{D} are independently selected from H, Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

or any two R^{C} groups together with the atom or atoms to which they are attached form a ring selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{Cy};

or any two R^{D} groups together with the atom or atoms to which they are attached form a ring selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{Cy};

Cy is selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{Cy};

each R^{Cy} is independently selected from H, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; each R^{a1}, R^{b1}, R^{c1}, and R^{d1} is independently selected from Cy¹, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy¹, halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2};

or any R^{c1} and R^{d1} together with the N atom to which they are attached form a 4-7 membered heterocycloalkyl, which is optionally substituted with 1, 2, or 3 substituents independently selected from R^{g};

Cy¹ is selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{Cy1};

each R^{Cy1} is independently selected from H, halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, S(O)₂NR^{c2}R^{d2}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, C(O)R_{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2};

each R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c2} and R^{d2} together with the N atom to which they are attached form a 4-7 membered heterocycloalkyl, which is optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆alkyl)aminosulfonyl, aminosulfonylamino, C₁₋₆ alkylaminosulfonylamino, di(C₁₋₆alkyl)aminosulfonylamino, aminocarbonylamino, C₁₋₆ alkylaminocarbonylamino, and di(C₁₋₆alkyl)aminocarbonylamino.

In some embodiments, R¹ is O.

In some embodiments, R¹ is S.

In some embodiments, R¹ is selected from N—OH, N—C₁₋₃ alkoxy, N—NH₂, and N—CN.

In some embodiments, W is C(O)OH.

In some embodiments, W is selected from C(O)NR^{c1}R^{d1}, C(O)NR^{c1}S(O)₂R^{b1}, C(O)NR^{c1}OR^{a1}, NR^{c1}C(O)OR^{a1}, NR^{c1}C(O)NR^{c1}R^{d1}, NR^{c1}C(O)R^{b1}, OR^{a1}, NR^{c1}R^{d1}, NR^{c1}S(O)₂R^{b1}, B(OH)₂, P(═O)(OR^{a1})₂.

In some embodiments, W is selected from halo, CN, and Cy.

In some embodiments, W is a carboxylic acid bioisostere.

In some embodiments, the carboxylic acid bioisostere is any one of chemical groups provided in Ballatore et al. “Carboxylic Acid (Bio)Isosteres in Drug Design”, ChemMedChem, 2013, 8(3), 385-395.

In some embodiments, the carboxylic acid bioisostere has any one of the following formulae:

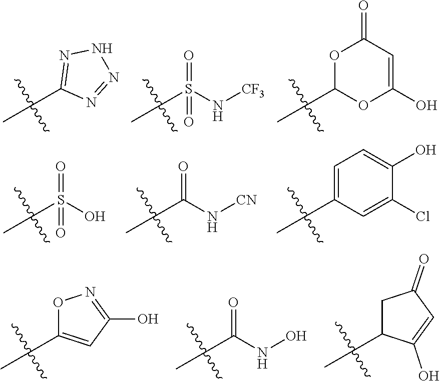

In some embodiments, X is N.

In some embodiments, X is CR².

In some embodiments, R² is selected from H, halo, and C₁₋₆ alkyl.

In some embodiments, R² is H.

In some embodiments, R² is halo (e.g., fluoro or chloro).

In some embodiments, Y is N.

In some embodiments, Y is CR³.

In some embodiments, R³ is selected from H, halo, and C₁₋₆ alkyl.

In some embodiments, R³ is H.

In some embodiments, R³ is halo (e.g., fluoro or chloro).

In some embodiments, ring A is a monocyclic C₃₋₇ cycloalkyl ring.

In some embodiments, ring A is a monocyclic 4-7 membered heterocycloalkyl ring.

In some embodiments, ring A is a phenyl ring.

In some embodiments, ring A is a monocyclic 5-6 membered heteroaryl ring.

In some embodiments, ring A is selected from a monocyclic 5-7 membered heterocycloalkyl ring and a monocyclic 5-6 membered heteroaryl ring.

In some embodiments, ring A is selected from pyridinyl, imidazolyl, pyrazolyl, triazolyl, piperidinyl, and dihydropyrazinyl.

In some embodiments, R^{A} is selected from Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R^{A} is selected from Cy, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R^{A} is Cy.

In some embodiments, R^{A} is C₁₋₆ alkyl, optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, each R^{A} is independently selected from halo, OH, C₁₋₆ alkyl, C₁₋₆ haloalkyl, C₁₋₆ alkoxy, C₁₋₆ haloalkoxy, and Cy, wherein said C₁₋₆ alkyl and C₁₋₆ alkoxy are each optionally substituted with OH, C₁₋₆ alkoxy, or Cy.

In some embodiments, each R^{A} is independently selected from halo, C₁₋₆ alkyl, C₁₋₆ haloalkyl, C₁₋₆ alkoxy, and C₁₋₆ haloalkoxy.

In some embodiments, each R^{A} is independently selected from C₁₋₆ alkyl and C₁₋₆ alkoxy. In some embodiments, R^{A} is halo. In some embodiments, R^{A} is C₁₋₆ alkoxy.

In some embodiments, R^{A} is C₁₋₆ alkyl, optionally substituted with 1, 2, or 3 substituents independently selected from halo and OR^{a1}.

In some embodiments, R^{A} is C₁₋₆ alkyl.

In some embodiments, R^{A} is selected from isopropyl and tert-butyl.

In some embodiments, ring B is selected from a monocyclic C₃₋₇ cycloalkyl ring, a monocyclic 4-7 membered heterocycloalkyl ring, and a phenyl ring.

In some embodiments, ring B is a monocyclic C₃₋₇ cycloalkyl ring.

In some embodiments, ring B is a monocyclic 4-7 membered heterocycloalkyl ring.

In some embodiments, ring B is a phenyl ring.

In some embodiments, ring B is a monocyclic 5-6 membered heteroaryl ring.

In some embodiments, R^{B} is selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}NR^{c1}S(O)₂R^{b1}, S(O)₂R^{b1}, and S(O)₂NR^{c1}R^{d1}.

In some embodiments, R^{B} is selected from Cy, halo, CN, OR^{a1}, NR^{c1}R^{d1}, S(O)₂R^{b1}, C₁₋₆ alkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

R^{B} is selected from Cy, halo, OR^{a1}, C(O)R^{b1}, and C₂₋₆ alkynyl, which is optionally substituted with Cy¹.

In some embodiments, R^{B} is OR^{a1}.

In some embodiments, R^{B} is Cy.

In some embodiments, R^{B} is halo.

In some embodiments, R^{B} is C₁₋₆ alkyl optionally substituted with OH or C₁₋₆ alkoxy.

In some embodiments, each R^{Cy} is independently selected from halo, CN, NO₂, OH, C₁₋₆ alkyl, C₁₋₆ haloalkyl, C₁₋₆ alkoxy, C₁₋₆ haloalkoxy, C(O)NH₂, C(O)OH, NH₂, and S(O)₂NH₂, wherein said C₁₋₆ alkyl is optionally substituted with 1, 2, or 3 substituents independently selected from halo, CN, NO₂, OH, C₁₋₆ alkyl, C₁₋₆ haloalkyl, C₁₋₆ alkoxy, C₁₋₆ haloalkoxy, C(O)NH₂, C(O)OH, NH₂, and S(O)₂NH₂.

In some embodiments, each R^{Cy} is independently selected from halo, CN, NO₂, OH, C₁₋₆ alkyl, C₁₋₆ haloalkyl, C₁₋₆ alkoxy, and C₁₋₆ haloalkoxy.

In some embodiments, R^{Cy} is selected from halo, CN, NO₂, OH, amino, C₁₋₆ alkyl, C₁₋₆ haloalkyl, C₁₋₆ alkoxy, and C₁₋₆ haloalkoxy.

In some embodiments, each R^{a1}, R^{b1}, R^{c1} and R^{d1} is independently selected from Cy¹, C₁₋₆ alkyl, and C₂₋₆ alkynyl, wherein said C₁₋₆ alkyl and C₂₋₆ alkynyl are each optionally substituted with 1, 2, or 3 substituents independently selected from Cy¹, halo, CN, NO₂, OR^{a2}, C(O)R^{b2}, (O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}.

In some embodiments, R^{a1} is selected from C₁₋₆ alkyl, optionally substituted with Cy¹ or OR^{a2}.

In some embodiments, R^{Cy1} is selected from halo, CN, and NR^{c2}R^{d2}.

In some embodiments, Cy¹ is selected from C₃₋₁₀ cycloalkyl, 5-10 membered heteroaryl, and 4-10 membered heterocycloalkyl.

In some embodiments, the compound of Formula (I) has formula:

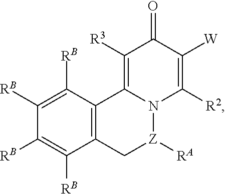

or a pharmaceutically acceptable salt thereof, wherein Z is selected from N and CR^{A}.

In some embodiments, the compound of Formula (I) has formula:

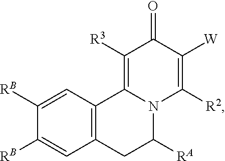

or a pharmaceutically acceptable salt thereof.

In some embodiments, the compound of Formula (I) has formula:

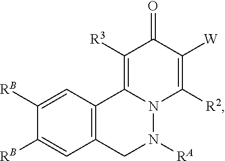

or a pharmaceutically acceptable salt thereof, wherein:

R^{A} is selected from Cy, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R^{A} is selected from Cy and C₁₋₆ alkyl, wherein said C₁₋₆ alkyl is optionally substituted with 1, 2, or 3 substituents independently selected from halo and OR^{a1}.

In some embodiments, the compound of Formula (I) is any one of compounds described in WO2018022282 and US20180170925, the content of which is incorporated herein by reference in their entirety.

In some embodiments, the compound of Formula (I) is:

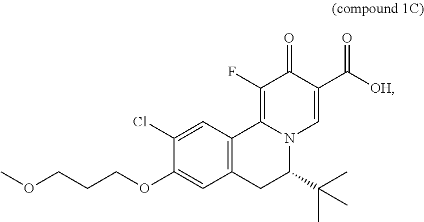 [SMILES: COCCCOc1cc2c(cc1Cl)-c1c(F)c(=O)c(C(=O)O)cn1[C@H](C(C)(C)C)C2]

or a pharmaceutically acceptable salt thereof.

In some embodiments, the compound of Formula (I) is:

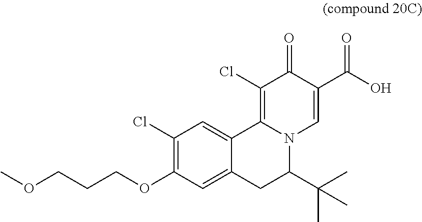 [SMILES: COCCCOc1cc2c(cc1Cl)-c1c(Cl)c(=O)c(C(=O)O)cn1C(C(C)(C)C)C2]

or a pharmaceutically acceptable salt thereof.

In some embodiments, the compound of Formula (I) is any one of compounds described in WO2018219356, the content of which is incorporated herein by reference in their entirety.

In some embodiments, the compound of Formula (I) is selected from any one of the following compounds:

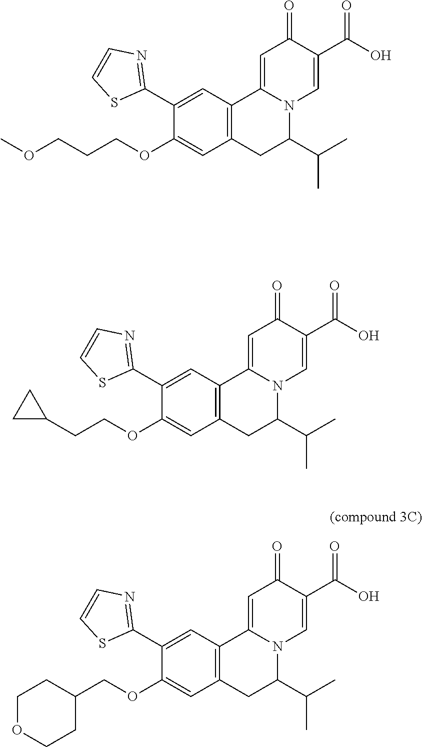 [SMILES: COCCCOc1cc2c(cc1-c1nccs1)-c1cc(=O)c(C(=O)O)cn1C(C(C)C)C2 ; CC(C)C1Cc2cc(OCCC3CC3)c(-c3nccs3)cc2-c2cc(=O)c(C(=O)O)cn21 ; CC(C)C1Cc2cc(OCC3CCOCC3)c(-c3nccs3)cc2-c2cc(=O)c(C(=O)O)cn21]

or a pharmaceutically acceptable salt thereof.

In some embodiments, the compound of Formula (I) is any one of compounds described in US20170342068 and WO2017205115, the content of which is incorporated herein by reference in their entirety.

In some embodiments, the compound of Formula (I) is:

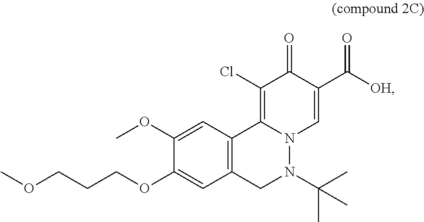 [SMILES: COCCCOc1cc2c(cc1OC)-c1c(Cl)c(=O)c(C(=O)O)cn1N(C(C)(C)C)C2]

or a pharmaceutically acceptable salt thereof.

In some embodiments, the compound of Formula (I) is any one of compounds described in US20160122344, WO2015173164, WO2016128335, WO2017013046, WO2017017043, and WO2017102648, the content of which is incorporated herein by reference in their entirety.

In some embodiments, the compound of Formula (I) has formula:

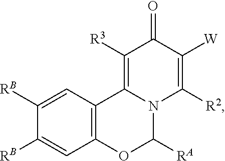

or a pharmaceutically acceptable salt thereof.

In some embodiments, each R^{B} is OR^{a1}.

In some embodiments, each R^{B} is independently selected from Cy, halo, OR^{a1}, and C₁₋₆ alkyl or C₂₋₆ alkynyl, each of which is optionally substituted with Cy¹, OR^{a2}, and S(O)₂R^{b2}.

In some embodiments, the compound of Formula (I) is any one of compounds described in WO2018161960, the content of which is incorporated herein by reference in its entirety.

In some embodiments, the compound of Formula (I) is selected from any one of the following compounds:

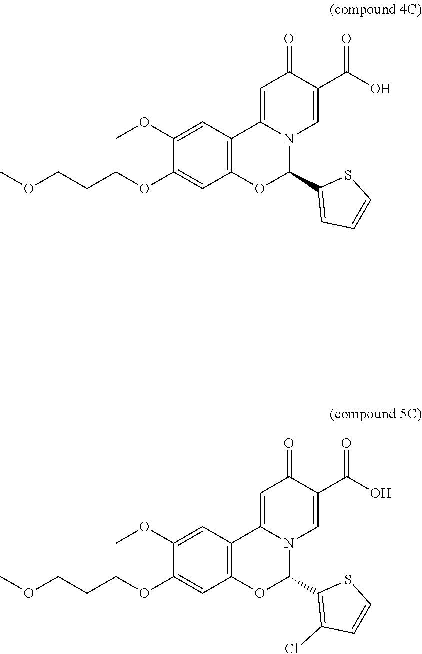 [SMILES: COCCCOc1cc2c(cc1OC)-c1cc(=O)c(C(=O)O)cn1[C@@H](c1cccs1)O2 ; COCCCOc1cc2c(cc1OC)-c1cc(=O)c(C(=O)O)cn1[C@H](c1sccc1Cl)O2]

or a pharmaceutically acceptable salt thereof.

In some embodiments, the compound of Formula (I) is

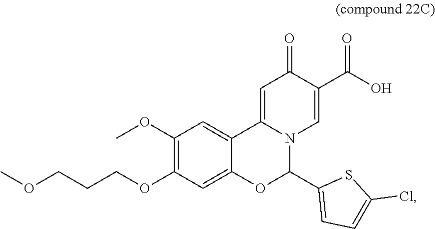 [SMILES: COCCCOc1cc2c(cc1OC)-c1cc(=O)c(C(=O)O)cn1C(c1ccc(Cl)s1)O2]

or a pharmaceutically acceptable salt thereof.

In some embodiments, the compound of Formula (I) has formula:

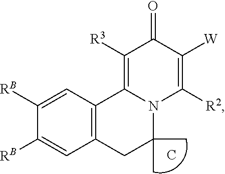

or a pharmaceutically acceptable salt thereof.

In some embodiments, the compound of Formula (I) is any one of compounds described in WO2018154466, the content of which is incorporated herein by reference in its entirety.

In some embodiments, the compound of Formula (I) is any one of compounds described in WO2018019297 and CN108727378, the content of which is incorporated herein by reference in its entirety.

In some embodiments, the compound of Formula (I) has formula:

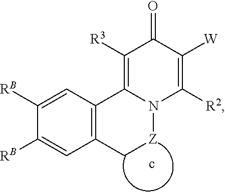

or a pharmaceutically acceptable salt thereof, wherein Z is selected from N and CR^{A}.

In some embodiments, Z is N.

In some embodiments, Z is CH.

In some embodiments, each R^{B} is OR^{a1}.

In some embodiments, each R^{B} is independently selected from Cy, halo, OR^{a1}, and C₁₋₆ alkyl or C₂₋₆ alkynyl, each of which is optionally substituted with Cy¹, OR^{a2}, and S(O)₂R^{b2}.

In some embodiments, ring C is C₃₋₇ cycloalkyl. In some embodiments, ring C is cyclopentyl. In some embodiments, ring C is cyclohexyl. In some aspects of these embodiments, R^{C} is C₁₋₆ alkyl.

In some embodiments, ring C is 4-7 membered heterocycloalkyl. In some embodiments, ring C is tetrahydrofuranyl.

In some embodiments, the compound of Formula (I) is any one of compounds described in US20180251460 and WO2018144605, the content of which is incorporated herein by reference in their entirety.

In some embodiments, the compound of Formula (I) is any one of compounds described in WO2017140821, U.S. Ser. No. 10/093,673, and CN106928245, the content of which is incorporated herein by reference in their entirety.

In some embodiments, the compound of Formula (I) is any one of compounds described in WO2017017042, the content of which is incorporated herein by reference in its entirety.

In some embodiments, the compound of Formula (I) is selected from:

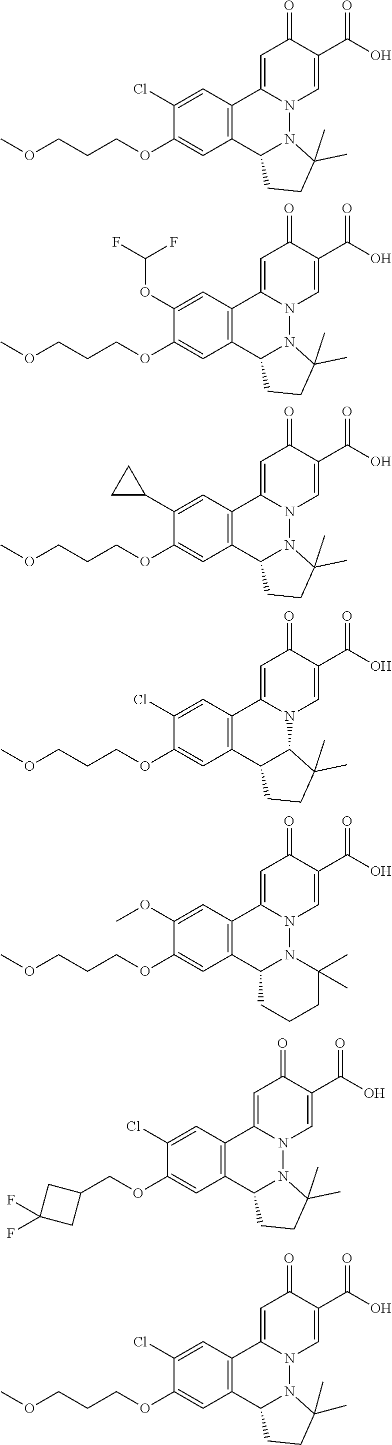

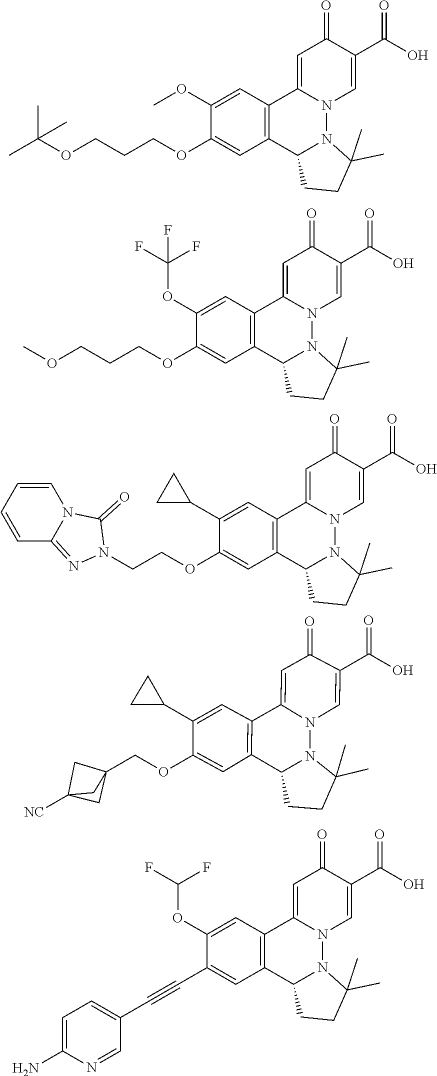

or a pharmaceutically acceptable salt thereof.

In some embodiments, the compound of Formula (I) is selected from:

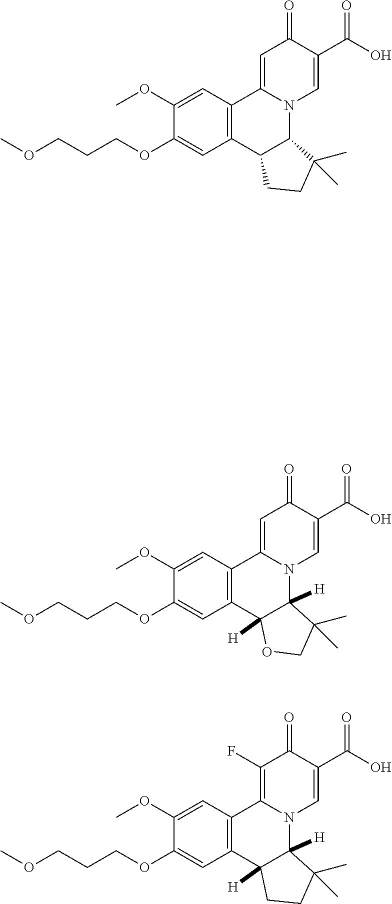 [SMILES: COCCCOc1cc2c(cc1OC)-c1cc(=O)c(C(=O)O)cn1[C@H]1[C@@H]2CCC1(C)C ; COCCCOc1cc2c(cc1OC)-c1cc(=O)c(C(=O)O)cn1[C@H]1[C@@H]2OCC1(C)C ; COCCCOc1cc2c(cc1OC)-c1c(F)c(=O)c(C(=O)O)cn1[C@H]1[C@@H]2CCC1(C)C]

or a pharmaceutically acceptable salt thereof.

In some embodiments, the compound of Formula (I) has formula:

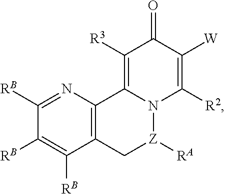

or a pharmaceutically acceptable salt thereof, wherein Z is selected from N and CR^{A}.

In some embodiments, the compound of Formula (I) has formula:

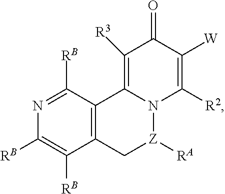

or a pharmaceutically acceptable salt thereof, wherein Z is selected from N and CR^{A}.

In some embodiments, the compound of Formula (I) has formula:

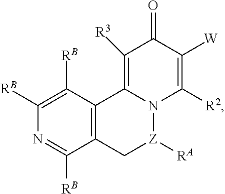

or a pharmaceutically acceptable salt thereof, wherein Z is selected from N and CR^{A}.

In some embodiments, the compound of Formula (I) has formula:

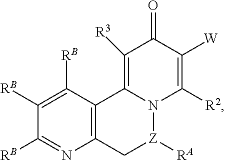

or a pharmaceutically acceptable salt thereof, wherein Z is selected from N and CR^{A}.

In some embodiments, the compound of Formula (I) has formula:

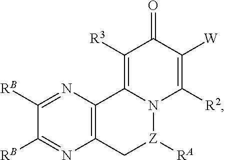

or a pharmaceutically acceptable salt thereof, wherein Z is selected from N and CR^{A}.

In some embodiments, the compound of Formula (I) has formula:

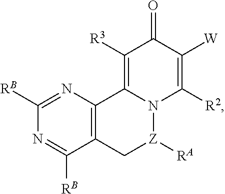

or a pharmaceutically acceptable salt thereof, wherein Z is selected from N and CR^{A}.

In some embodiments, the compound of Formula (I) has formula:

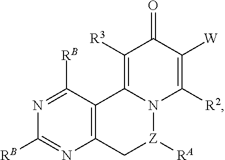

or a pharmaceutically acceptable salt thereof, wherein Z is selected from N and CR^{A}.

In some embodiments, Z is N.

In some embodiments, Z is CR^{A}, and R^{A} is C₁₋₆ alkyl.

In some embodiments, Z is CH.

In some embodiments, R^{A} is selected from Cy, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R^{A} is Cy.

In some embodiments, R^{A} is selected from Cy and C₁₋₆ alkyl, wherein said C₁₋₆ alkyl is optionally substituted with 1, 2, or 3 substituents independently selected from halo and OR^{a1}.

In some embodiments, R^{A} is C₁₋₆ alkyl, substituted with OR^{a1}. In some embodiments, each R^{B} is OR^{a1}.

In some embodiments, each R^{B} is independently selected from Cy, halo, OR^{a1}, and C₁₋₆ alkyl or C₂₋₆ alkynyl, each of which is optionally substituted with Cy¹, OR^{a2}, and S(O)₂R^{b2}.

In some embodiments, R² is H.

In some embodiments, R³ is halo.

In some embodiments, R³ is F.

In some embodiments, the compound of Formula (I) is any one of compounds described in WO2018085619, the content of which is incorporated herein by reference in their entirety.

In some embodiments, the compound of Formula (I) is any one of compounds described in WO2018047109, the content of which is incorporated herein by reference in their entirety.

In some embodiments, the compound of Formula (I) is any one of the following compounds:

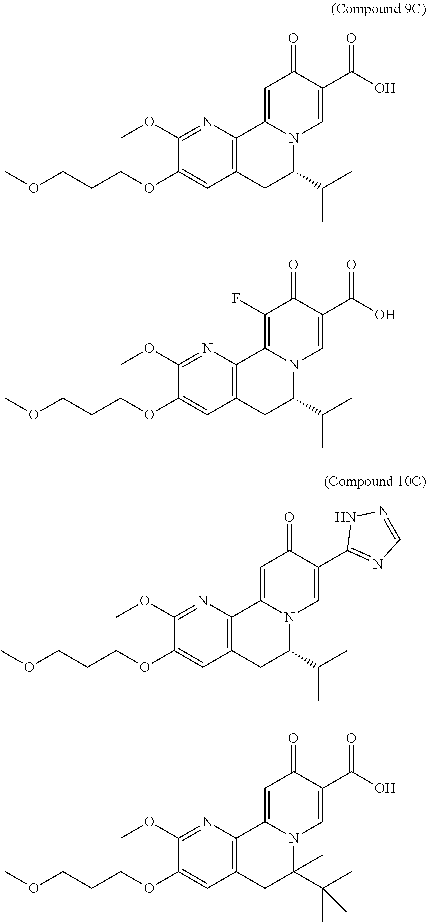

or a pharmaceutically acceptable salt thereof.

In some embodiments, the compound of Formula (I) is:

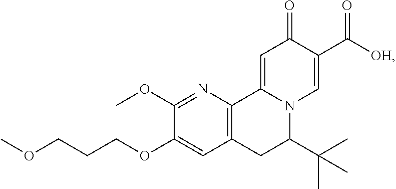 [SMILES: COCCCOc1cc2c(nc1OC)-c1cc(=O)c(C(=O)O)cn1C(C(C)(C)C)C2]

or a pharmaceutically acceptable salt thereof.

In some embodiments, the compound of Formula (I) has formula:

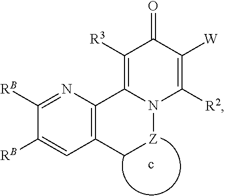

or a pharmaceutically acceptable salt thereof, wherein Z is selected from N and CR^{A}.

In some embodiments, the compound of Formula (I) has formula:

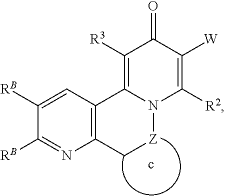

or a pharmaceutically acceptable salt thereof, wherein Z is selected from N and CR^{A}.

In some embodiments, Z is N.

In some embodiments, Z is CH.

In some embodiments, each R^{B} is OR^{a1}.

In some embodiments, each R^{B} is independently selected from Cy, halo, OR^{a1}, and C₁₋₆ alkyl or C₂₋₆ alkynyl, each of which is optionally substituted with Cy¹, OR^{a2}, and S(O)₂R^{b2}.

In some embodiments, ring C is C₃₋₇ cycloalkyl. In some embodiments, ring C is cyclopentyl. In some embodiments, ring C is cyclohexyl. In some aspect of these embodiments, R^{C} is C₁₋₆ alkyl.

In some embodiments, ring C is 4-7 membered heterocycloalkyl. In some embodiments, ring C is tetrahydrofuranyl.

In some embodiments, the compound of Formula (I) is any one of compounds described in WO2018047109, the content of which is incorporated herein by reference in their entirety.

In some embodiments, the compound of Formula (I) is any one of the following compounds:

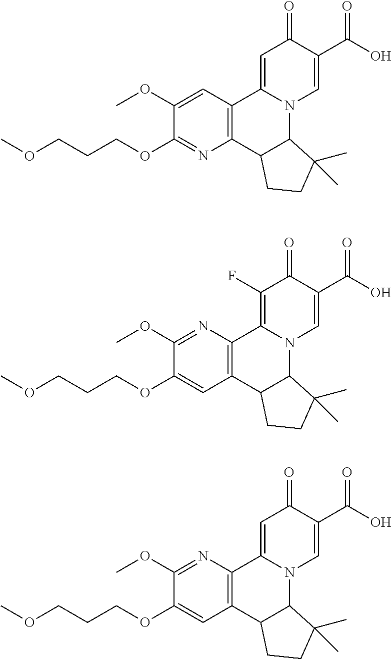

or a pharmaceutically acceptable salt thereof.

In some embodiments, the compound of Formula (I) is any one of the following compounds:

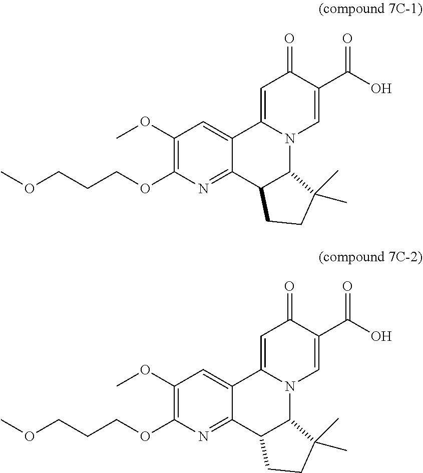

or a pharmaceutically acceptable salt thereof.

In some embodiments, the compound of Formula (I) has formula:

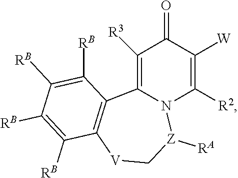

or a pharmaceutically acceptable salt thereof, wherein V is selected from O, NR^{A}, and C(R^{A})₂, and Z is selected from N and CR^{A}.

In some embodiments, the compound of Formula (I) has formula:

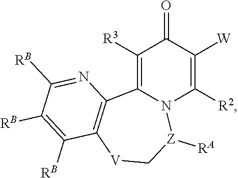

or a pharmaceutically acceptable salt thereof, wherein V is selected from O, NR^{A}, and C(R^{A})₂, and Z is selected from N and CR^{A}.

In some embodiments, the compound of Formula (I) has formula:

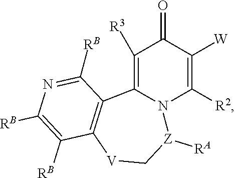

or a pharmaceutically acceptable salt thereof, wherein V is selected from O, NR^{A}, and C(R^{A})₂, and Z is selected from N and CR^{A}.

In some embodiments, the compound of Formula (I) has formula:

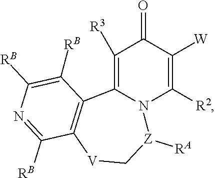

or a pharmaceutically acceptable salt thereof, wherein V is selected from O, NR^{A}, and C(R^{A})₂, and Z is selected from N and CR^{A}.

In some embodiments, the compound of Formula (I) has formula:

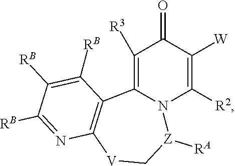

or a pharmaceutically acceptable salt thereof, wherein V is selected from O, NR^{A}, and C(R^{A})₂, and Z is selected from N and CR^{A}.

In some embodiments, the compound of Formula (I) has formula:

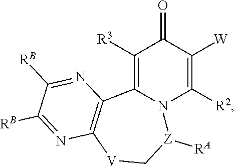

or a pharmaceutically acceptable salt thereof, wherein V is selected from O, NR^{A}, and C(R^{A})₂, and Z is selected from N and CR^{A}.

In some embodiments, the compound of Formula (I) has formula:

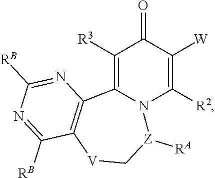

or a pharmaceutically acceptable salt thereof, wherein V is selected from O, NR^{A}, and C(R^{A})₂, and Z is selected from N and CR^{A}.

In some embodiments, the compound of Formula (I) has formula:

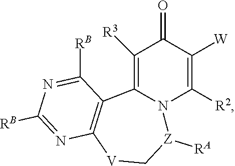

or a pharmaceutically acceptable salt thereof, wherein V is selected from O, NR^{A}, and C(R^{A})₂, and Z is selected from N and CR^{A}.

In some embodiments, V is O.

In some embodiments, V is CH₂.

In some embodiments, V is NR^{A}.

In some embodiments, Z is N.

In some embodiments, Z is CH.

In some embodiments, R^{A} is selected from Cy, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R^{A} is Cy.

In some embodiments, R^{A} is selected from Cy and C₁₋₆ alkyl, wherein said C₁₋₆ alkyl is optionally substituted with 1, 2, or 3 substituents independently selected from halo and OR^{a1}.

In some embodiments, R^{A} is C₁₋₆ alkyl, substituted with OR^{a1}.

In some embodiments, each R^{B} is OR^{a1}.

In some embodiments, each R^{B} is independently selected from Cy, halo, OR^{a1}, and C₁₋₆ alkyl or C₂₋₆ alkynyl, each of which is optionally substituted with Cy¹, OR^{a2}, and S(O)₂R^{b2}.

In some embodiments, R² is H.

In some embodiments, R³ is halo.

In some embodiments, R³ is F.

In some embodiments, the compound of Formula (I) is any one of compounds described in WO2018085619, the content of which is incorporated herein by reference in its entirety.

In some embodiments, the compound of Formula (I) is any one of the following compounds:

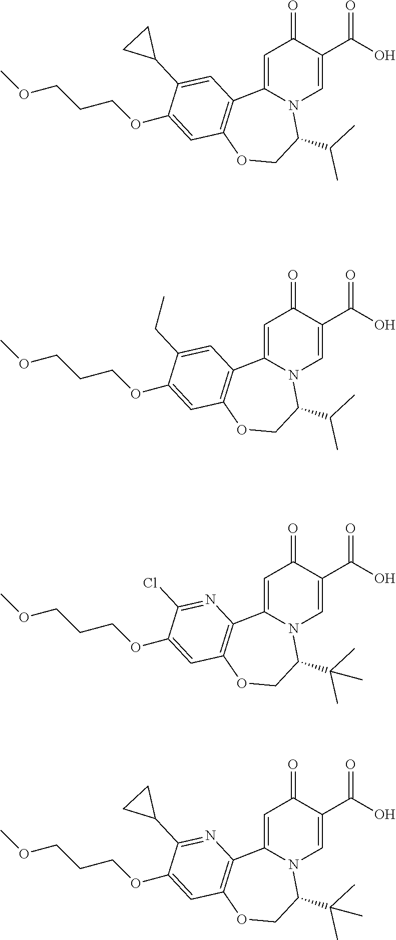 [SMILES: COCCCOc1cc2c(cc1C1CC1)-c1cc(=O)c(C(=O)O)cn1[C@H](C(C)C)CO2 ; CCc1cc2c(cc1OCCCOC)OC[C@@H](C(C)C)n1cc(C(=O)O)c(=O)cc1-2 ; COCCCOc1cc2c(nc1Cl)-c1cc(=O)c(C(=O)O)cn1[C@H](C(C)(C)C)CO2 ; COCCCOc1cc2c(nc1C1CC1)-c1cc(=O)c(C(=O)O)cn1[C@H](C(C)(C)C)CO2]

or a pharmaceutically acceptable salt thereof.

In some embodiments, the compound of Formula (I) is any one of compounds described in WO2018214875, the content of which is incorporated herein by reference in its entirety.

In some embodiments, the compound of Formula (I) is any one of the following compounds:

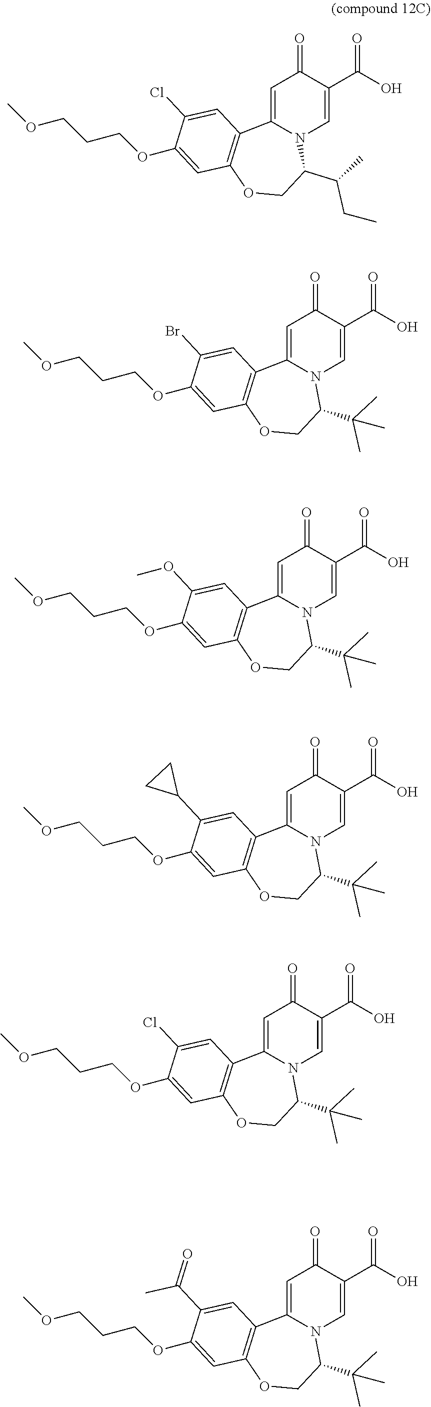 [SMILES: CC[C@@H](C)[C@@H]1COc2cc(OCCCOC)c(Cl)cc2-c2cc(=O)c(C(=O)O)cn21 ; COCCCOc1cc2c(cc1Br)-c1cc(=O)c(C(=O)O)cn1[C@H](C(C)(C)C)CO2 ; COCCCOc1cc2c(cc1OC)-c1cc(=O)c(C(=O)O)cn1[C@H](C(C)(C)C)CO2 ; COCCCOc1cc2c(cc1C1CC1)-c1cc(=O)c(C(=O)O)cn1[C@H](C(C)(C)C)CO2 ; COCCCOc1cc2c(cc1Cl)-c1cc(=O)c(C(=O)O)cn1[C@H](C(C)(C)C)CO2 ; COCCCOc1cc2c(cc1C(C)=O)-c1cc(=O)c(C(=O)O)cn1[C@H](C(C)(C)C)CO2]

or a pharmaceutically acceptable salt thereof.

In some embodiments, the compound of Formula (I) has formula:

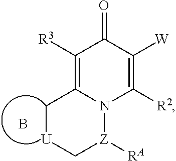

or a pharmaceutically acceptable salt thereof, wherein U is selected from N, C, and CR^{A}, and Z is selected from N and CR^{A}.

In some embodiments, U is N.

In some embodiments, U is C.

In some embodiments, Z is N.

In some embodiments, Z is CH.

In some embodiments, the compound of Formula (I) has formula:

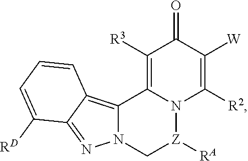

or a pharmaceutically acceptable salt thereof.

In some embodiments, the compound of Formula (I) has formula:

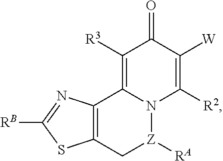

or a pharmaceutically acceptable salt thereof, wherein Z is selected from N and CR^{A}.

In some embodiments, the compound of Formula (I) has formula:

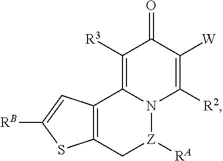

or a pharmaceutically acceptable salt thereof, wherein Z is selected from N and CR^{A}.

In some embodiments, the compound of Formula (I) is any one of compounds described in WO2018198079 and US20180312507, the content of which is incorporated herein by reference in their entirety.

In some embodiments, the compound of Formula (I) is any one of compounds described in WO2018085619, the content of which is incorporated herein by reference in their entirety.

Compounds of Formula (II)

In some embodiments, the compound of Formula (I) has Formula (II):

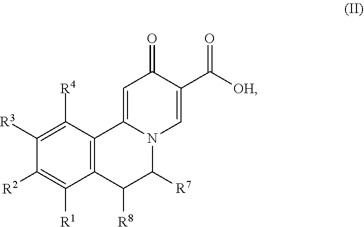

or a pharmaceutically acceptable salt thereof, wherein R¹, R², R³, R⁴, R⁷, and R⁸ are as described herein.

Certain embodiments of the Formula (II) are described below:

In some embodiments:

R¹ is selected from H, C₁₋₆ alkyl, halo, CN, and OR^{a1};

R² is selected from H, C₁₋₆ alkyl, C₁₋₄ haloalkyl, Cy¹, halo, CN, OR^{a1}, and NR^{c1}R^{d1}.

R³ is selected from H, C₁₋₆ alkyl, C₁₋₄ haloalkyl, halo, and OR^{a1};

R⁴ is selected from H, C₁₋₆ alkyl, halo, OR^{a1}, and NR^{c1}R^{d1},

R⁷ is selected from H, C₁₋₆ alkyl, C₁₋₄ haloalkyl, Cy¹, and halo; wherein said C₁₋₆ alkyl is optionally substituted with Cy¹;

R⁸ is selected from H and C₁₋₆ alkyl;

R^{a1}, R^{c1}, and R^{d1} are each independently selected from H, C₁₋₆ alkyl, C₂₋₆ alkenyl, and C₂₋₆ alkynyl; wherein said C₁₋₆ alkyl, C₂₋₆ alkenyl, and C₂₋₆ alkynyl are each optionally substituted with 1, 2, or 3 substituents independently selected from Cy³, halo, CN, OR^{a3}, C(O)R^{b3}, C(O)OR^{a3}, NR^{c3}R^{d3}, NR^{c3}S(O)₂R^{b3}, S(O)R^{b3}, and S(O)₂R^{b3};

R^{a3}, R^{c3}, and R^{d3} are each independently selected from H, C₁₋₆ alkyl, C(O)R^{b4}, and C(O)OR^{a4}; wherein said C₁₋₆ alkyl is optionally substituted with OR^{a4} or NR^{c4}R^{d4};

R^{b3} is selected from C₁₋₆ alkyl and 4-12 membered heterocycloalkyl;

each Cy¹ is independently selected from C₆₋₁₀ aryl, C₃₋₁₀ cycloalkyl, 5-10 membered heteroaryl, and 4-12 membered heterocycloalkyl, each of which is optionally substituted with 1, 2, or 3 substituents independently selected from R^{Cy1};

each Cy³ is independently selected from C₆₋₁₀ aryl, C₃₋₁₀ cycloalkyl, 5-10 membered heteroaryl, and 4-12 membered heterocycloalkyl, each of which is optionally substituted with 1, 2, or 3 substituents independently selected from R^{Cy3};

R^{Cy1} and R^{Cy3} are each independently selected from halo, C₁₋₄ alkyl, CN, and C(O)OR^{a4},

R^{a4}, R^{c4}, and R^{d4} are each independently selected from H and C₁₋₆ alkyl; and

each R^{b4} is C₁₋₆ alkyl.

In some embodiments, R¹ is H. In some embodiments, R¹ is C₁₋₆ alkyl. In some embodiments, R¹ is halo. In some embodiments, R¹ is CN. In some embodiments, R¹ is OR^{a1}. In some embodiments, le is selected from H, C₁₋₆ alkyl, and halo. In some embodiments, R¹ is selected from H and C₁₋₆ alkyl.

In some embodiments, R² is H. In some embodiments, R² is C₁₋₆ alkyl. In some embodiments, R² is C₁₋₄ haloalkyl. In some embodiments, R² is Cy¹. In some embodiments, R² is halo or CN. In some embodiments, R² is OR^{a1}. In some embodiments, R² is NR^{c1}R^{d1}. In some embodiments, R² is selected from H, C₁₋₆ alkyl, and C₁₋₄ haloalkyl. In some embodiments, R² is selected from OR^{a1} and NR^{c1}R^{d1}.

In some embodiments, R³ is H. In some embodiments, R³ is C₁₋₆ alkyl. In some embodiments, R³ is C₁₋₄ haloalkyl. In some embodiments, R³ is halo. In some embodiments, R³ is OR^{a1}.

In some embodiments, R⁴ is H. In some embodiments, R⁴ is C₁₋₆ alkyl. In some embodiments, R⁴ is C₁₋₄ haloalkyl. In some embodiments, R⁴ is halo. In some embodiments, R⁴ is OR^{a1}. In some embodiments, R⁴ is NR^{c1}R^{d1}. In some embodiments, R⁴ is selected from OR^{a1} and NR^{c1}R^{d1}.

In some embodiments, R⁷ is H. In some embodiments, R⁷ is C₁₋₆ alkyl. In some embodiments, R⁷ is Cy¹. In some embodiments, R⁷ is halo. In some embodiments, R⁷ is C₁₋₆ alkyl substituted with Cy¹.

In some embodiments, R⁸ is H. In some embodiments, R⁸ is C₁₋₆ alkyl.

In some embodiments, R^{a1} is H. In some embodiments, R^{a1} is C₁₋₆ alkyl. In some embodiments, R^{a1} is C₂₋₆ alkenyl. In some embodiments, R^{a1} is C₂₋₆ alkynyl. In some embodiments, R^{a1} is C₁₋₆ alkyl substituted with 1, 2, or 3 substituents independently selected from Cy³, halo, CN, OR^{a3}, C(O)R^{b3}, C(O)OR^{a3}, NR^{c3}R^{d3}, NR^{c3}S(O)₂R^{b3}, S(O)R^{b3}, and S(O)₂R^{b3}. In some embodiments, R^{a1} is C₁₋₆ alkyl substituted with Cy³. In some embodiments, R^{a1} is C₁₋₆ alkyl substituted with 1, 2, or 3 halo. In some embodiments, R^{a1} is C₂₋₆ alkenyl substituted with 1, 2, or 3 halo. In some embodiments, R^{a1} is C₂₋₆ alkynyl substituted with 1, 2, or 3 halo. In some embodiments, R^{a1} is C₁₋₆ alkyl substituted OR^{a3}. In some embodiments, R^{a1} is C₁₋₆ alkyl substituted with halo, CN, OR^{a3}, C(O)R^{b3}, C(O)ORa³, NR^{c3}R^{d3}, NR^{c3}S(O)₂R^{b3}, S(O)R^{b3}, or S(O)₂R^{b3}. In some embodiments, R^{a1} is C₁₋₆ alkyl substituted Cy³. In some embodiments, R^{a1} is C₂₋₆ alkenyl or C₂₋₆ alkynyl, substituted OR^{a3}.

In some embodiments, R^{c1} and R^{d1} are each H.

In some embodiments, R^{c1} and R^{d1} are each independently H or C₁₋₆ alkyl. In some embodiments, at least one of R^{c1} and R^{d1} is C₁₋₆ alkyl substituted with 1, 2, or 3 substituents independently selected from Cy³, halo, CN, OR^{a3}, C(O)R^{b3}, C(O)OR^{a3}, NR^{c3}R^{d3}, NR^{c3}S(O)₂R^{b3}, S(O)R^{b3}, and S(O)₂R^{b3}. In some embodiments, at least one of R^{c1} and R^{d1} is C₁₋₆ alkyl substituted with Cy³. In some embodiments, at least one of R^{c1} and R^{d1} is C₁₋₆ alkyl substituted with halo, CN, OR^{a3}, C(O)R^{b3}, C(O)OR^{a3}, NR^{c3}R^{d3}, NR^{c3}S(O)₂R^{b3}, S(O)R^{b3}, or S(O)₂R^{b3}.

In some embodiments, R^{a3} is H. In some embodiments, R^{a3} is C₁₋₆ alkyl. In some embodiments, R^{a3} is C₁₋₆ alkyl substituted with OR^{a4} or NR^{c4}R^{d4.} In some embodiments, R^{a3} is C₁₋₆ alkyl substituted with OR^{a4}. In some embodiments, R^{a3} is C₁₋₆ alkyl substituted with NR^{c4}R^{d4}.

In some embodiments, R^{c3} and R^{d3} are each H.

In some embodiments, R^{c3} and R^{d3} are each independently H or C₁₋₆ alkyl. In some embodiments, at least one of R^{c3} and R^{d3} is C₁₋₆ alkyl substituted with OR^{a4}. In some embodiments, at least one of R^{c3} and R^{d3} is C₁₋₆ alkyl substituted with NR^{c4}R^{d4}. In some embodiments, at least one of R^{c3} and R^{d3} is C(O)R^{b4}. In some embodiments, at least one of R^{c3} and R^{d3} is C(O)OR^{a4}.

In some embodiments, R^{b3} is C₁₋₆ alkyl. In some embodiments, R^{b3} is 4-12 membered heterocycloalkyl (e.g., morpholinyl, piperidinyl, pyrrolidinyl, or piperazinyl).

In some embodiments, Cy¹ is C₆₋₁₀ aryl (e.g., phenyl or naphthyl), optionally substituted with 1, 2, or 3 substituents independently selected from R^{Cy1}.

In some embodiments, Cy¹ is C₃₋₁₀ cycloalkyl (e.g., cyclopropyl, cyclobutyl, cyclopentyl, or cyclohexyl), optionally substituted with 1, 2, or 3 substituents independently selected from R^{Cy1}.

In some embodiments, Cy¹ is 5-10 membered heteroaryl (e.g., pyridinyl, pyrrolidinyl, oxazolyl, isoxazolyl, or pyrazinyl), optionally substituted with 1, 2, or 3 substituents independently selected from R^{Cy1}.

In some embodiments, Cy¹ is 4-12 membered heterocycloalkyl (e.g., morpholinyl, piperidinyl, pyrrolidinyl, or piperazinyl), optionally substituted with 1, 2, or 3 substituents independently selected from R^{Cy1}.

In some embodiments, Cy³ is C₆₋₁₀ aryl (e.g., phenyl or naphthyl), optionally substituted with 1, 2, or 3 substituents independently selected from R^{Cy3}.

In some embodiments, Cy³ is C₃₋₁₀ cycloalkyl (e.g., cyclopropyl, cyclobutyl, cyclopentyl, or cyclohexyl), optionally substituted with 1, 2, or 3 substituents independently selected from R^{Cy3}.

In some embodiments, Cy³ is 5-10 membered heteroaryl (e.g., pyridinyl, pyrrolidinyl, oxazolyl, isoxazolyl, or pyrazinyl), optionally substituted with 1, 2, or 3 substituents independently selected from R^{Cy3}.

In some embodiments, Cy³ is 4-12 membered heterocycloalkyl (e.g., morpholinyl, piperidinyl, pyrrolidinyl, or piperazinyl), optionally substituted with 1, 2, or 3 substituents independently selected from R^{Cy3}.

In some embodiments, R^{Cy1} is halo. In some embodiments, R^{Cy1} is C₁₋₄ alkyl. In some embodiments, R^{Cy1} is CN. In some embodiments, R^{Cy1} is C(O)OR^{a4}.

In some embodiments, R^{Cy3} is halo. In some embodiments, R^{Cy3} is C₁₋₄ alkyl. In some embodiments, R^{Cy3} is CN. In some embodiments, R^{Cy3} is C(O)OR^{a4}.

In some embodiments, R^{a4} is H. In some embodiments, R^{a4} is C₁₋₆ alkyl.

In some embodiments, R^{c4} and R^{d4} are each H. In some embodiments, one of R^{c4} and R^{d4} is H, and the other is C₁₋₆ alkyl.

In some embodiments, R^{b4} is C₁₋₆ alkyl.

In some embodiments, 6-methyl-2-oxo-9-pyrrolidin-1-yl-6,7-dihydrobenzo[a]quinolizine-3-carboxylic acid, 9-fluoro-6-methyl-2-oxo-6,7-dihydrobenzo[a]quinolizine-3-carboxylic acid, and 9,10-difluoro-6-methyl-2-oxo-6,7-dihydrobenzo[a]quinolizine-3-carboxylic acid are excluded from the scope of the compound of Formula (II).

In some embodiments, R¹, R², R³ and R⁴ are not all H simultaneously.

In some embodiments:

R⁴ is hydrogen, fluoro, chloro, bromo, methyl, methylamino, methoxy or ethoxy;

R³ is hydrogen, fluoro, chloro, bromo, methyl, ethyl, trifluoromethyl, methoxy, ethoxy, propoxy, trifluoromethoxy, cyano, cyclopropyl, hydroxy or phenylmethyl-O—;

R² is hydrogen, bromo, methyl, propyl, trifluoromethyl, cyano, phenylmethyl-N(methyl)-, tert-butoxycarbonylpiperazinyl, hydroxy, methoxy, ethoxy, propoxy, isopropoxy, butoxy, isobutoxy, difluoromethylmethyl-O—, difluoromethylethyl-O—, trifluoromethoxy, trifluoromethylmethyl-O—, trifluoromethylethyl-O—, ethyldifluoromethyl-O—, vinyldifluoromethyl-O—, propargyl-O—, hydroxymethylpropargyl-O—, methoxyethyl-O—, methoxypropyl-O—, methoxybutyl-O—, ethoxyethyl-O—, methoxyethyl-O-ethyl-O—, aminoethyl-O—, aminopentyl-O—, aminohexyl-O—, aminooctyl-O—, tert-butoxycarbonylaminopentyl-O—, tert-butoxycarbonylaminohexyl-O—, tert-butoxycarbonylaminooctyl-O—, methylcarbonylaminoethyl-O—, methylcarbonylaminopentyl-O—, methylsulfonylaminoethyl-O—, methylsulfonylaminopentyl-O—, methyl sulfonylethyl-O—, methylsulfonylpropyl-O—, methyl sulfanylpropyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-dimethylpropyl-O—, hydroxy-difluoropropyl-O—, hydroxybutyl-O—, hydroxypentyl-O—, hydroxyhexyl-O—, aminoethyl-O-propyl-O—, ethylamino-ethyl-O-propyl-O—, imidazolylethyl-O—, pyrazolylpropyl-O—, triazolylpropyl-O—, morpholinylethyl-O—, morpholinylpropyl-O—, (2-oxo-pyrrolidinyl)ethyl-O—, (2-oxo-pyrrolidinyl)propyl-O—, phenylmethyl-O—, phenylethyl-O—, pyrrolidinylethyl-O—, pyrrolidinylpropyl-O—, pyrrolidinylcarbonylmethyl-O—, tetrahydropyranylmethyl-O— or carboxypropyl-O—;

R¹ is hydrogen, fluoro, chloro, bromo, methyl or cyano;

R⁸ is hydrogen or methyl; and

R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl.

In some embodiments:

R⁴ is hydrogen, halogen, C₁₋₆ alkylamino or C₁₋₆ alkoxy;

R³ is hydrogen, halogen, C₁₋₆ alkyl, C₁₋₆ alkoxy, C₃₋₇ cycloalkyl, hydroxy or phenyl-CₓH₂ₓ—O—;

R² is hydrogen; halogen; C₁₋₆ alkyl; cyano; phenyl-CₓH₂ₓ—N(C₁₋₆ alkyl)-; C₁₋₆ alkoxycarbonylpiperazinyl; or R^{a1}—O—, wherein R^{a1} is hydrogen; C₁₋₆ alkyl, which is unsubstituted or substituted with one to three substituents independently selected from fluoro, hydroxy and C₂₋₆alkenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; C₃₋₇cycloalkylC₁₋₆alkyl; cyanoC₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; heteroarylC₁₋₆alkyl (e.g., heteroaryl is N-containing monocyclic heteroaryl); or heterocycloalkylC₁₋₆alkyl (e.g., heterocycloalkyl is monocyclic heterocycloalkyl);

R¹ is hydrogen, halogen, C₁₋₆alkyl or cyano;

R⁸ is hydrogen or C₁₋₆alkyl;

R⁷ is hydrogen; C₁₋₆alkyl, which is unsubstituted or once, twice or three times substituted by fluoro; C₃₋₇cycloalkyl; C₁₋₆alkylC₃₋₇cycloalkyl; or phenyl-CₓH₂ₓ—; and

x is 1-6.

In some embodiments, 9-fluoro-6-methyl-2-oxo-6,7-dihydrobenzo[a]quinolizine-3-carboxylic acid and 9,10-difluoro-6-methyl-2-oxo-6,7-dihydrobenzo[a]quinolizine-3-carboxylic acid are excluded from the scope of the compounds of Formula (II).

In some embodiments:

R⁴ is hydrogen, fluoro, chloro, bromo, methylamino, methoxy or ethoxy;

R³ is hydrogen, fluoro, chloro, methyl, ethyl, methoxy, ethoxy, propoxy, cyclopropyl, hydroxy or phenylmethyl-O—;

R² is hydrogen, bromo, methyl, propyl, cyano, phenylmethyl-N(methyl)-, tert-butoxycarbonylpiperazinyl, hydroxy, methoxy, ethoxy, propoxy, isopropoxy, butoxy, isobutoxy, difluoromethylmethyl-O—, difluoromethylethyl-O—, trifluoromethylmethyl-O—, ethyldifluoromethyl-O—, vinyldifluoromethyl-O—, propargyl-O—, hydroxymethylpropargyl-O—, methoxyethyl-O—, methoxypropyl-O—, methoxybutyl-O—, ethoxyethyl-O—, methoxyethyl-O-ethyl-O—, aminoethyl-O—, aminopentyl-O—, aminohexyl-O—, aminooctyl-O—, tert-butoxycarbonylaminopentyl-O—, tert-butoxycarbonylaminohexyl-O—, tert-butoxycarbonylaminooctyl-O—, methylcarbonylaminoethyl-O—, methylcarbonylaminopentyl-O—, methylsulfonylaminoethyl-O—, methylsulfonylaminopentyl-O—, methyl sulfonylethyl-O—, methylsulfonylpropyl-O—, methyl sulfanylpropyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-dimethylpropyl-O—, hydroxy-difluoropropyl-O—, hydroxybutyl-O—, hydroxypentyl-O—, hydroxyhexyl-O—, aminoethyl-O-propyl-O—, ethylamino-ethyl-O-propyl-O—, imidazolylethyl-O—, pyrazolylpropyl-O—, triazolylpropyl-O—, morpholinylethyl-O—, morpholinylpropyl-O—, (2-oxo-pyrrolidinyl)ethyl-O—, (2-oxo-pyrrolidinyl)propyl-O—, phenylmethyl-O—, phenylethyl-O—, pyrrolidinylethyl-O—, pyrrolidinylpropyl-O—, pyrrolidinylcarbonylmethyl-O—, tetrahydropyranylmethyl-O— or carboxypropyl-O—;

R¹ is hydrogen, chloro, bromo, methyl or cyano;

R⁸ is hydrogen or methyl; and

R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl.

In some embodiments, the compound of Formula (II) has Formula (IIB):

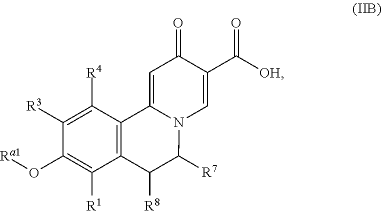

or a pharmaceutically acceptable salt thereof, wherein:

R⁴ is hydrogen, halogen or C₁₋₆alkoxy;

R³ is hydrogen, halogen, C₁₋₆alkyl, C₁₋₆alkoxy, C₃₋₇cycloalkyl, hydroxy or phenyl-CₓH₂ₓ—O—;

R¹ is hydrogen or halogen;

R⁸ is hydrogen or C₁₋₆alkyl;

R⁷ is hydrogen; C₁₋₆alkyl, which is unsubstituted or once, twice or three times substituted by fluoro; C₃₋₇cycloalkyl; C₁₋₆alkylC₃₋₇cycloalkyl; or phenyl-CₓH₂ₓ—;

R^{a1} is hydrogen; C₁₋₆alkyl, which is unsubstituted or substituted with one to three substituents independently selected from fluoro, hydroxy and ethenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; C₃₋₇cycloalkylC₁₋₆alkyl; cyanoC₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; heteroarylC₁₋₆alkyl (e.g., heteroaryl is N-containing monocyclic heteroaryl); or heterocycloalkylC₁₋₆alkyl (e.g., heterocycloalkyl is monocyclic heterocycloalkyl); and

x is 1-6.

In some embodiments:

R⁴ is hydrogen, fluoro, chloro or methoxy;

R³ is hydrogen, fluoro, chloro, methyl, ethyl, methoxy, ethoxy, propoxy, cyclopropyl, hydroxy or phenylmethyl-O—;

R¹ is hydrogen or chloro;

R⁸ is hydrogen or methyl;

R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl; and

R^{a1} is hydrogen, methyl, ethyl, propyl, isopropyl, butyl, isobutyl, difluoromethylmethyl, difluoromethyl ethyl, trifluoromethylmethyl, ethyldifluoromethyl, vinyldifluoromethyl, propargyl, hydroxymethylpropargyl, methoxyethyl, methoxypropyl, methoxybutyl, ethoxyethyl, methoxyethyl-O-ethyl, aminoethyl, aminopentyl, aminohexyl, aminooctyl, tert-butoxycarbonylaminopentyl, tert-butoxycarbonylaminohexyl, tert-butoxycarbonylaminooctyl, methylcarbonylaminoethyl, methylcarbonylaminopentyl, methyl sulfonylaminoethyl, methyl sulfonylaminopentyl, methylsulfonylethyl, methyl sulfonylpropyl, methyl sulfanylpropyl, cyanopropyl, cyanocyclopropylmethyl, cyclopropylmethyl, cyclohexylethyl, hydroxyethyl, hydroxypropyl, hydroxy-dimethylpropyl, hydroxy-difluoropropyl, hydroxybutyl, hydroxypentyl, hydroxyhexyl, aminoethyl-O-propyl, ethylamino-ethyl-O-propyl-, imidazolylethyl, pyrazolylpropyl, triazolylpropyl, morpholinyl ethyl, morpholinylpropyl, (2-oxo-pyrrolidinyl)ethyl, (2-oxo-pyrrolidinyl)propyl, phenylmethyl, phenylethyl, pyrrolidinylethyl, pyrrolidinylpropyl, pyrrolidinylcarbonylmethyl, tetrahydropyranylmethyl or carboxypropyl.

In some embodiments:

R⁴ is hydrogen or halogen;

R³ is C₁₋₆alkyl, halogen or C₃₋₇cycloalkyl;

R¹ is hydrogen;

R⁸ is hydrogen or C₁₋₆alkyl;

R⁷ is C₁₋₆alkyl or C₁₋₆alkylC₃₋₇cycloalkyl; and

R^{a1} is C₁₋₆alkyl, C₁₋₆alkoxyC₁₋₆alkyl or phenylC₁₋₆alkyl.

In some embodiments:

R⁴ is hydrogen, fluoro or chloro;

R³ is methyl, ethyl, fluoro, chloro or cyclopropyl;

R¹ is hydrogen;

R⁸ is hydrogen or methyl;

R⁷ is methyl, ethyl, isopropyl, isobutyl, tert-butyl or methylcyclopropyl; and

R^{a1} is methyl, ethyl, methoxyethyl, methoxypropyl or phenylmethyl.

In some embodiments:

R⁴ is hydrogen;

R³ is C₁₋₆alkoxy;

R¹ is hydrogen or halogen;

R⁸ is hydrogen or C₁₋₆alkyl;

R⁷ is hydrogen; C₁₋₆alkyl, which is unsubstituted or once, twice or three times substituted by fluoro; C₃₋₇cycloalkyl; C₁₋₆alkylC₃₋₇cycloalkyl; or phenyl-CₓH₂ₓ—;

R^{a1} is hydrogen; C₁₋₆alkyl, which is unsubstituted or substituted with one to three substituents independently selected from fluoro, hydroxy and C₂₋₆alkenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; cyanoC₃₋₇cycloalkylC₁₋₆alkyl; C₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; imidazolylC₁₋₆alkyl; pyrazolylC₁₋₆alkyl; triazolylC₁₋₆alkyl; morpholinylC₁₋₆alkyl; (2-oxo-pyrrolidinyl)C₁₋₆ alkyl; pyrrolidinylC₁₋₆ alkyl; or tetrahydropyranylC₁₋₆ alkyl; and

x is 1-6.

In some embodiments:

R⁴ is hydrogen;

R³ is methoxy, ethoxy or propoxy;

R¹ is hydrogen or chloro;

R⁸ is hydrogen or methyl;

R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl; and

R^{a1} is hydrogen, methyl, ethyl, propyl, isopropyl, butyl, isobutyl, difluoromethylmethyl, difluoromethylethyl, trifluoromethylmethyl, ethyldifluoromethyl, vinyldifluoromethyl, propargyl, hydroxymethylpropargyl, methoxyethyl, methoxypropyl, methoxybutyl, ethoxyethyl, methoxyethyl-O-ethyl, aminoethyl, aminopentyl, aminohexyl, aminooctyl, tert-butoxycarbonylaminopentyl, tert-butoxycarbonylaminohexyl, tert-butoxycarbonylaminooctyl, methylcarbonylaminoethyl, methylcarbonylaminopentyl, methyl sulfonylaminoethyl, methyl sulfonylaminopentyl, methyl sulfonyl ethyl, methyl sulfonylpropyl, methyl sulfanylpropyl, cyanopropyl, cyanocyclopropylmethyl, cyclopropylmethyl, cyclohexylethyl, hydroxyethyl, hydroxypropyl, hydroxy-dimethylpropyl, hydroxy-difluoropropyl, hydroxybutyl, hydroxypentyl, hydroxyhexyl, aminoethyl-O-propyl, ethylamino-ethyl-O-propyl-, imidazolylethyl, pyrazolylpropyl, triazolylpropyl, morpholinylethyl, morpholinylpropyl, (2-oxo-pyrrolidinyl)ethyl, (2-oxo-pyrrolidinyl)propyl, phenylmethyl, phenylethyl, pyrrolidinylethyl, pyrrolidinylpropyl, pyrrolidinylcarbonylmethyl, tetrahydropyranylmethyl or carboxypropyl.

In some embodiments:

R⁴ is hydrogen or halogen;

R³ is halogen, C₁₋₆alkyl, C₁₋₆alkoxy or C₃₋₇cycloalkyl;

R¹ is hydrogen;

R⁸ is hydrogen or C₁₋₆alkyl;

R⁷ is C₁₋₆alkyl, which is unsubstituted or once, twice or three times substituted by fluoro; C₃₋₇cycloalkyl or C₁₋₆alkylC₃₋₇cycloalkyl; and

R^{a1} is C₁₋₆alkyl, which is unsubstituted or substituted with one to three substituents independently selected from fluoro and hydroxy; C₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆ alkylsulfanylC₁₋₆alkyl; C₁₋₆alkylsulfonylC₁₋₆alkyl; C₃₋₇cycloalkylC₁₋₆ alkyl; phenylC₁₋₆alkyl; C₁₋₆alkylaminoC₁₋₆alkoxyC₁₋₆alkyl; C₁₋₆alkoxycarbonylaminoC₁₋₈alkyl; morpholinylC₁₋₆ alkyl or tetrahydropyranylC₁₋₆ alkyl.

In some embodiments:

R⁴ is hydrogen, fluoro, or chloro;

R³ is fluoro, chloro, methyl, ethyl, methoxy, ethoxy or cyclopropyl;

R¹ is hydrogen;

R⁸ is hydrogen or methyl;

R⁷ is methyl, ethyl, isopropyl, isobutyl, tert-butyl, trifluoromethylmethyl, cyclobutyl or methylcyclopropyl; and

R^{a1} is methyl, ethyl, propyl, butyl, isobutyl, cyclopropylmethyl, difluoromethylmethyl, difluoromethylethyl, trifluoromethylmethyl, ethyldifluoromethyl, methoxyethyl, methoxypropyl, ethoxyethyl, aminohexyl, aminooctyl, tert-butoxycarbonylaminopentyl, tert-butoxycarbonylaminooctyl, methylcarbonylaminopentyl, methyl sulfonylaminopentyl, methyl sulfonylpropyl, methyl sulfanylpropyl, hydroxypropyl, hydroxy-dimethylpropyl, hydroxy-difluoropropyl, hydroxybutyl, hydroxypentyl, hydroxyhexyl, ethylamino-ethyl-O-propyl-, morpholinylethyl, morpholinylpropyl, phenylmethyl or tetrahydropyranylmethyl.

In some embodiments, R⁴ is hydrogen. In some embodiments, R³ is halogen or C₁₋₆alkoxy. In some embodiments, R³ is chloro or methoxy. In some embodiments, R⁸ is hydrogen. In some embodiments, R⁷ is C₁₋₆ alkyl or C₁₋₆ alkylC₃₋₇cycloalkyl. In some embodiments, R⁷ is ethyl, isopropyl, tert-butyl or methylcyclopropyl. In some embodiments, R^{a1} is C₁₋₆alkoxyC₁₋₆alkyl, hydroxyC₁₋₆alkyl or aminoC₁₋₆alkyl. In some embodiments, R^{a1} is methoxyethyl, methoxypropyl, hydroxydimethylpropyl, hydroxybutyl, hydroxypentyl, hydroxyhexyl, aminobutyl, aminopentyl or aminohexyl.

In some embodiments:

R⁴ is hydrogen, halogen, C₁₋₆alkylamino or C₁₋₆alkoxy;

R³ is hydrogen, C₁₋₆alkyl or C₁₋₆alkoxy;

R² is hydrogen; halogen; C₁₋₆alkyl; cyano; C₁₋₆alkoxycarbonylpiperazinyl or phenyl-CₓH₂ₓ—N(C₁₋₆alkyl)-, wherein x is 1-8;

R¹ is hydrogen, halogen, C₁₋₆alkyl or cyano;

R⁸ is hydrogen; and

R⁷ is C₁₋₆alkyl;

or a pharmaceutically acceptable salt thereof.

In some embodiments:

R⁴ is hydrogen, bromo, methylamino or ethoxy;

R³ is hydrogen, methyl or methoxy;

R² is hydrogen, bromo, methyl, propyl, cyano, tert-butoxycarbonylpiperazinyl or phenylmethyl-N(methyl)-;

R¹ is hydrogen, bromo, methyl or cyano;

R⁸ is hydrogen; and

R⁷ is methyl or ethyl.

In some embodiments:

R⁴ is hydrogen, halogen, C₁₋₆alkyl, C₁₋₆alkylamino or C₁₋₆alkoxy;

R³ is hydrogen; halogen; C₁₋₆alkyl, which is unsubstituted or once or more times substituted by fluoro; C₁₋₆alkoxy, which is unsubstituted or once or more times substituted by fluoro; cyano; C₃₋₇cycloalkyl; hydroxy or phenyl-CₓH₂ₓ—O—;

R² is hydrogen; halogen; C₁₋₆alkyl, which is unsubstituted or once or more times substituted by fluoro; cyano; morpholinyl; pyrrolidinyl; phenyl-CₓH₂ₓ—N(C₁₋₆alkyl)-; C₁₋₆ alkoxycarbonylpiperazinyl; or R^{a1}—O—; wherein

R^{a1} is hydrogen; C₁₋₆alkyl, which is unsubstituted or once or more times substituted by fluoro; or R^{2A}—CₓH₂ₓ—; wherein R^{2A} is Cy³, halo, CN, OR^{a3}, C(O)R^{b3}, C(O)OR^{a3}, NR^{c3}R^{d3}, NR^{c3}S(O)₂R^{b3}, S(O)R^{b3}, or S(O)₂R^{b3};

R⁷ is hydrogen; C₁₋₆alkyl, which is unsubstituted or once or more times substituted by fluoro; C₃₋₇cycloalkyl or C₃₋₇cycloalkyl-CₓH₂ₓ—.

x is 1-6;

R¹ is hydrogen, halogen, C₁₋₆alkyl or cyano; and

R⁸ is hydrogen or C₁₋₆alkyl.

In some embodiments, R^{2A} is C₁₋₆alkoxy, C₁₋₆alkylcarbonylamino, C₁₋₆alkylsulfonylamino, C₁₋₆alkylsulfonyl, aminocarbonyl, cyano, cyanoC₃₋₇cycloalkyl, C₃₋₇cycloalkyl, diC₁₋₆alkylamino, hydroxy, imidazolyl, morpholinyl, 2-oxo-pyrrolidinyl, phenyl, pyrrolidinyl, pyrrolidinylcarbonyl or tetrahydropyranyl.

In some embodiments, 6-methyl-2-oxo-9-pyrrolidin-1-yl-6,7-dihydrobenzo[a]quinolizine-3-carboxylic acid, 9-fluoro-6-methyl-2-oxo-6,7-dihydrobenzo[a]quinolizine-3-carboxylic acid, and 9,10-difluoro-6-methyl-2-oxo-6,7-dihydrobenzo[a]quinolizine-3-carboxylic acid are excluded from the scope of Formula (II).

In some embodiments:

R⁴ is hydrogen, fluoro, chloro, bromo, methyl, methylamino, methoxy or ethoxy;

R³ is hydrogen, fluoro, chloro, bromo, methyl, ethyl, trifluoromethyl, methoxy, ethoxy, propoxy, trifluoromethoxy, cyano, cyclopropyl, hydroxy or phenylmethyl-O—;

R² is hydrogen, bromo, methyl, propyl, trifluoromethyl, cyano, morpholinyl, pyrrolidinyl, phenylmethyl-N(methyl)-, tert-butoxycarbonylpiperazinyl, hydroxy, methoxy, ethoxy, propoxy, isopropoxy, isobutoxy, butoxy, difluoromethylmethyl-O—, difluoromethylethyl-O—, trifluoromethoxy, trifluoromethylmethyl-O—, trifluoromethylethyl-O—, methoxyethyl-O—, methoxypropyl-O—, ethoxyethyl-O—, methoxyethyl-O-ethyl-O—, methyl carbonyl aminoethyl-O—, methylsulfonylaminoethyl-O—, methylsulfonylethyl-O—, aminocarbonylmethyl-O—, cyanomethyl-O—, cyanopropyl-O—, cyanocyclopropylmethyl-O—, cyclopropylmethyl-O—, cyclohexylethyl-O—, diethylaminoethyl-O—, hydroxyethyl-O—, hydroxypropyl-O—, hydroxy-2,2-dimethylpropyl-O—, imidazolylethyl-O—, morpholinylethyl-O—, 2-oxo-pyrrolidin-1-ylethyl-O—, phenylmethyl-O—, phenylethyl-O—, pyrrolidinylethyl-O—, pyrrolidinylcarbonylmethyl-O— or tetrahydropyran-4-ylmethyl-O—;

R¹ is hydrogen, fluoro, chloro, bromo, methyl or cyano;

R⁸ is hydrogen or methyl; and

R⁷ is hydrogen, methyl, ethyl, propyl, isopropyl, isobutyl, trifluoromethyl, trifluoroethyl, cyclopropyl, cyclobutyl or cyclopropylmethyl.

In some embodiments, 6-methyl-2-oxo-9-pyrrolidin-1-yl-6,7-dihydrobenzo[a]quinolizine-3-carboxylic acid is excluded from the scope of Formula (II).

In some embodiments:

R⁴ is hydrogen, halogen, C₁₋₆alkylamino or C₁₋₆alkoxy;

R³ is hydrogen, halogen, C₁₋₆alkyl, C₁₋₆alkoxy, C₃₋₇cycloalkyl, hydroxy or phenyl-CₓH₂ₓ—O—;

R² is hydrogen; halogen; C₁₋₆alkyl; cyano; phenyl-CₓH₂ₓ—N(C₁₋₆alkyl)-; C₁₋₆alkoxycarbonylpiperazinyl; or R^{a1}—O—; wherein R^{a1} is hydrogen; C₁₋₆ alkyl, which is unsubstituted or once or more times substituted by fluoro; or R^{2A}—CₓH₂ₓ—; wherein R^{2A} is C₁₋₆alkoxy, C₁₋₆alkoxy-CₓH₂ₓ—O—, C₁₋₆alkylcarbonylamino, C₁₋₆alkylsulfonylamino, C₁₋₆alkylsulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl;

R¹ is hydrogen, halogen, C₁₋₆alkyl or cyano;

R⁸ is hydrogen or C₁₋₆alkyl;

R⁷ is hydrogen; C₁₋₆alkyl, which is unsubstituted or once or more times substituted by fluoro; or C₃₋₇cycloalkyl; and

x is 1-6.

In some embodiments, 9-fluoro-6-methyl-2-oxo-6,7-dihydrobenzo[a]quinolizine-3-carboxylic acid and 9,10-difluoro-6-methyl-2-oxo-6,7-dihydrobenzo[a]quinolizine-3-carboxylic acid are excluded from the scope of Formula (II).

In some embodiments:

R⁴ is hydrogen, chloro, bromo, methylamino, methoxy or ethoxy;

R³ is hydrogen, fluoro, chloro, methyl, ethyl, methoxy, ethoxy, propoxy, cyclopropyl, hydroxy or phenylmethyl-O—;

R² is hydrogen, bromo, methyl, propyl, cyano, phenylmethyl-N(methyl)-, tert-butoxycarbonylpiperazinyl, hydroxy, methoxy, ethoxy, propoxy, isopropoxy, isobutoxy, butoxy, difluoromethylmethyl-O—, trifluoromethylmethyl-O—, methoxyethyl-O—, methoxypropyl-O—, ethoxyethyl-O—, methoxyethyl-O-ethyl-O—, methylcarbonylaminoethyl-O—, methyl sulfonyl aminoethyl-O—, methyl sulfonylethyl-O—, cyanomethyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-2,2-dimethylpropyl-O—, imidazolylethyl-O—, morpholinylethyl-O—, 2-oxo-pyrrolidin-1-ylethyl-O—, phenylmethyl-O—, phenylethyl-O—, pyrrolidinylethyl-O—, pyrrolidinylcarbonylmethyl-O— or tetrahydropyran-4-ylmethyl-O—;

R¹ is hydrogen, chloro, bromo, methyl or cyano;

R⁸ is hydrogen or methyl; and

R⁷ is hydrogen, methyl, ethyl, propyl, isopropyl, isobutyl, trifluoromethyl or cyclopropyl.

In some embodiments, the compound of Formula (II) is a compound of Formula (IIB):

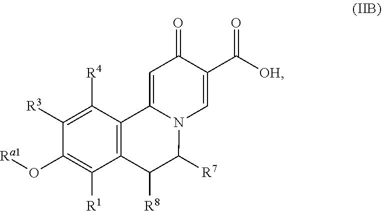

or a pharmaceutically acceptable salt thereof, wherein:

R⁴ is hydrogen, halogen or C₁₋₆alkoxy;

R³ is hydrogen, halogen, C₁₋₆alkyl, C₁₋₆ alkoxy, C₃₋₇cycloalkyl, hydroxy or phenyl-CₓH₂ₓ—O—;

R¹ is hydrogen or halogen;

R⁸ is hydrogen;

R⁷ is C₁₋₆alkyl, which is unsubstituted or once or more times substituted by fluoro; or C₃₋₇cycloalkyl;

R^{a1} is hydrogen; C₁₋₆alkyl, which is unsubstituted or once or more times substituted by fluoro; R^{2A}—CₓH₂ₓ—; wherein R^{2A} is C₁₋₆alkoxy, C₁₋₆alkylcarbonylamino, C₁₋₆alkylsulfonylamino, C₁₋₆alkylsulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl; and

x is 1-6.

In some embodiments:

R⁴ is hydrogen, chloro or methoxy;

R³ is hydrogen, fluoro, chloro, methyl, ethyl, methoxy, ethoxy, propoxy, cyclopropyl, hydroxy or phenylmethyl-O—;

R¹ is hydrogen or chloro;

R⁸ is hydrogen;

R⁷ is methyl, ethyl, propyl, isopropyl, isobutyl, trifluoromethyl or cyclopropyl; and

R^{a1} is hydrogen, methyl, ethyl, propyl, isopropyl, isobutyl, butyl, difluoroethyl, trifluoroethyl, methoxyethyl, methoxypropyl, ethoxyethyl, methoxyethyl-O-ethyl, methylcarbonylaminoethyl, methyl sulfonylaminoethyl, methylsulfonylethyl, cyanomethyl, cyanopropyl, cyanocyclopropylmethyl, cyclopropylmethyl, cyclohexylethyl, hydroxyethyl, hydroxypropyl, hydroxy-2,2-dimethylpropyl, imidazolylethyl, morpholinylethyl, 2-oxo-pyrrolidin-1-ylethyl, phenylmethyl, phenyl ethyl, pyrrolidinylethyl, pyrrolidinylcarbonylmethyl or tetrahydropyran-4-ylmethyl.

In some embodiments:

R⁴ is hydrogen;

R³ is halogen;

R¹ is hydrogen;

R⁸ is hydrogen;

R⁷ is C₁₋₆alkyl;

R^{a1} is C₁₋₆alkyl or C₁₋₆alkoxy-CₓH₂ₓ—; and

x is 1-6.

In some embodiments:

R⁴ is hydrogen;

R³ is C₁₋₆alkyl or C₃₋₇cycloalkyl;

R¹ is hydrogen;

R⁸ is hydrogen;

R⁷ is C₁₋₆alkyl;

R^{a1} is C₁₋₆alkyl or phenyl-CₓH₂ₓ—; and

x is 1-6.

In some embodiments:

R⁴ is hydrogen;

R³ is C₁₋₆alkoxy;

R¹ is hydrogen or halogen;

R⁸ is hydrogen;

R⁷ is C₁₋₆alkyl, which is unsubstituted or once or more times substituted by fluoro; or C₃₋₇cycloalkyl;

R^{a1} is hydrogen; C₁₋₆alkyl, which is unsubstituted or once or more times substituted by fluoro; or R^{2A}—CₓH₂ₓ—;

R^{2A} is C₁₋₆alkoxy, C₁₋₆alkoxy-CₓH₂ₓ—O—, C₁₋₆alkylcarbonylamino, C₁₋₆alkylsulfonylamino, C₁₋₆ alkyl sulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl; and

x is 1-6.

In some embodiments:

R⁴ is hydrogen;

R³ is methoxy, ethoxy or propoxy;

R¹ is hydrogen or chloro;

R⁸ is hydrogen;

R⁷ is methyl, ethyl, propyl, isopropyl, isobutyl, trifluoromethyl or cyclopropyl; and

R^{a1} is hydrogen, methyl, ethyl, propyl, isopropyl, isobutyl, butyl, difluoroethyl, trifluoroethyl, methoxyethyl, methoxypropyl, ethoxyethyl, methoxyethyl-O-ethyl, methylcarbonylaminoethyl, methyl sulfonylaminoethyl, methylsulfonylethyl, cyanomethyl, cyanopropyl, cyanocyclopropylmethyl, cyclopropylmethyl, cyclohexylethyl, hydroxyethyl, hydroxypropyl, hydroxy-2,2-dimethylpropyl, imidazolylethyl, morpholinylethyl, 2-oxo-pyrrolidin-1-ylethyl, phenylmethyl, phenyl ethyl, pyrrolidinylethyl, pyrrolidinylcarbonylmethyl or tetrahydropyran-4-ylmethyl.

In some embodiments:

R⁴ is hydrogen;

R³ is C₁₋₆alkoxy;

R² is C₁₋₆alkoxy;

R¹ is hydrogen;

R⁸ is hydrogen or C₁₋₆alkyl; and

R⁷ is hydrogen.

In some embodiments:

R⁴ is hydrogen, halogen, C₁₋₆alkylamino or C₁₋₆alkoxy;

R³ is hydrogen, C₁₋₆alkyl or C₁₋₆alkoxy;

R² is hydrogen, bromo, C₁₋₆ alkyl, C₁₋₆ alkoxycarbonylpiperazinyl, cyano or phenyl-CₓH₂ₓ—N(C₁₋₆alkyl)-; and

R¹ is hydrogen, halogen, C₁₋₆alkyl or cyano;

R⁸ is hydrogen;

R⁷ is C₁₋₆alkyl; and

x is 1-6.

In some embodiments:

R⁴ is hydrogen, bromo, methylamino or ethoxy;

R³ is hydrogen, methyl or methoxy;

R² is hydrogen, bromo, methyl, propyl, tert-butoxycarbonylpiperazinyl, cyano or phenylmethyl-N(methyl)-;

R¹ is hydrogen, bromo, methyl or cyano;

R⁸ is hydrogen; and

R⁷ is methyl or ethyl.

In some embodiments, the compound of Formula (II) is selected from:

9-Benzyloxy-10-methoxy-6-methyl-2-oxo-6,7-dihydrobenzo[a]quinolizine-3-carboxylic acid;

9-Hydroxy-10-methoxy-6-methyl-2-oxo-6,7-dihydrobenzo[a]quinolizine-3-carboxylic acid;

9,11-Dimethoxy-6-methyl-2-oxo-6,7-dihydrobenzo[a]quinolizine-3-carboxylic acid;

9-Ethoxy-10-methoxy-6-methyl-2-oxo-6,7-dihydrobenzo[a]quinoli zine-3-carboxylic acid;

9,10-Diethoxy-6-ethyl-2-oxo-6,7-dihydrobenzo[a]quinolizine-3-carboxylic acid;

(+)-9,10-Diethoxy-6-ethyl-2-oxo-6,7-dihydrobenzo[a]quinolizine-3-carboxylic acid;

(−)-9,10-Diethoxy-6-ethyl-2-oxo-6,7-dihydrobenzo[a]quinolizine-3-carboxylic acid;

9-Benzyloxy-6-ethyl-10-methoxy-2-oxo-6,7-dihydrobenzo[a]quinolizine-3-carboxylic acid;

(+)-9-Benzyloxy-6-ethyl-10-methoxy-2-oxo-6,7-dihydrobenzo[a]quinolizine-3-carboxylic acid;

(−)-9-Benzyloxy-6-ethyl-10-methoxy-2-oxo-6,7-dihydrobenzo[a]quinolizine-3-carboxylic acid;

(+)-6-Ethyl-10-methoxy-2-oxo-9-(2,2,2-trifluoroethoxy)-6,7-dihydrobenzo[a]quinolizine-3-carboxylic acid;

(−)-6-Ethyl-10-methoxy-2-oxo-9-(2,2,2-trifluoroethoxy)-6,7-dihydrobenzo[a]quinolizine-3-carboxylic acid;

6-Ethyl-9-isopropoxy-10-methoxy-2-oxo-6,7-dihydrobenzo[a]quinolizine-3-carboxylic acid;

6-Ethyl-10-methoxy-2-oxo-9-(2-phenylethoxy)-6,7-dihydrobenzo[a]quinolizine-3-carboxylic acid;

9-Butoxy-6-ethyl-10-methoxy-2-oxo-6,7-dihydrobenzo[a]quinolizine-3-carboxylic acid;

9-(2-Cyclohexylethoxy)-6-ethyl-10-methoxy-2-oxo-6,7-dihydrobenzo[a]quinolizine-3-carboxylic acid;

6-Ethyl-10-methoxy-2-oxo-9-prop-2-ynoxy-6,7-dihydrobenzo[a]quinolizine-3-carboxylic acid;

6-Ethyl-10-methoxy-2-oxo-9-(2-oxo-2-pyrrolidin-1-yl-ethoxy)-6,7-dihydrobenzo[a]quinolizine-3-carboxylic acid;

6-Ethyl-10-methoxy-9-[2-(2-methoxyethoxyl)ethoxy]-2-oxo-6,7-dihydrobenzo[a]quinolizine-3-carboxylic acid;

6-Ethyl-10-methoxy-9-(2-morpholinoethoxy)-2-oxo-6,7-dihydrobenzo[a]quinolizine-3-carboxylic acid;

6-Ethyl-9-(2-hydroxyethoxy)-10-methoxy-2-oxo-6,7-dihydrobenzo[a]quinolizine-3-carboxylic acid;

6-Ethyl-9-(3-hydroxy-2,2-dimethyl-propoxy)-10-methoxy-2-oxo-6,7-dihydrobenzo[a]quinolizine-3-carboxylic acid;

(+)-6-Ethyl-9-(3-hydroxy-2,2-dimethyl-propoxy)-10-methoxy-2-oxo-6,7-dihydrobenzo[a]quinolizine-3-carboxylic acid;

(−)-6-Ethyl-9-(3-hydroxy-2,2-dimethyl-propoxy)-10-methoxy-2-oxo-6,7-dihydrobenzo[a]quinolizine-3-carboxylic acid;

6-Ethyl-9-(3-hydroxypropoxy)-10-methoxy-2-oxo-6,7-dihydrobenzo[a]quinolizine-3-carboxylic acid;

6-Ethyl-9-(2-imidazol-1-ylethoxy)-10-methoxy-2-oxo-6,7-dihydrobenzo[a]quinolizine-3-carboxylic acid;

6-Ethyl-10-methoxy-9-(2-methoxyethoxy)-2-oxo-6,7-dihydrobenzo[a]quinolizine-3-carboxylic acid;

(+)-6-Ethyl-10-methoxy-9-(2-methoxyethoxy)-2-oxo-6,7-dihydrobenzo[a]quinolizine-3-carboxylic acid;

(−)-6-Ethyl-10-methoxy-9-(2-methoxyethoxy)-2-oxo-6,7-dihydrobenzo[a]quinolizine-3-carboxylic acid;

9-(Cyclopropylmethoxy)-6-ethyl-10-methoxy-2-oxo-6,7-dihydrobenzo[a]quinolizine-3-carboxylic acid;

(+)-9-(Cyclopropylmethoxy)-6-ethyl-10-methoxy-2-oxo-6,7-dihydrobenzo[a]quinolizine-3-carboxylic acid;

(−)-9-(Cyclopropylmethoxy)-6-ethyl-10-methoxy-2-oxo-6,7-dihydrobenzo[a]quinolizine-3-carboxylic acid;

6-Ethyl-9-isobutoxy-10-methoxy-2-oxo-6,7-dihydrobenzo[a]quinolizine-3-carboxylic acid;

(+)-6-Ethyl-9-isobutoxy-10-methoxy-2-oxo-6,7-dihydrobenzo[a]quinolizine-3-carboxylic acid;

(−)-6-Ethyl-9-isobutoxy-10-methoxy-2-oxo-6,7-dihydrobenzo[a]quinolizine-3-carboxylic acid;

9-Ethoxy-6-ethyl-10-methoxy-2-oxo-6,7-dihydrobenzo[a]quinolizine-3-carboxylic acid;

(+)-9-Ethoxy-6-ethyl-10-methoxy-2-oxo-6,7-dihydrobenzo[a]quinolizine-3-carboxylic acid;

(−)-9-Ethoxy-6-ethyl-10-methoxy-2-oxo-6,7-dihydrobenzo[a]quinolizine-3-carboxylic acid;

6-Ethyl-10-methoxy-9-(3-methoxypropoxy)-2-oxo-6,7-dihydrobenzo[a]quinolizine-3-carboxylic acid;

(+)-6-Ethyl-10-methoxy-9-(3-methoxypropoxy)-2-oxo-6,7-dihydrobenzo[a]quinolizine-3-carboxylic acid;

(−)-6-Ethyl-10-methoxy-9-(3-methoxypropoxy)-2-oxo-6,7-dihydrobenzo[a]quinolizine-3-carboxylic acid;

9-(2-Ethoxyethoxy)-6-ethyl-10-methoxy-2-oxo-6,7-dihydrobenzo[a]quinolizine-3-carboxylic acid;

(+)-9-(2-Ethoxyethoxy)-6-ethyl-10-methoxy-2-oxo-6,7-dihydrobenzo[a]quinolizine-3-carboxylic acid;

(−)-9-(2-Ethoxyethoxy)-6-ethyl-10-methoxy-2-oxo-6,7-dihydrobenzo[a]quinolizine-3-carboxylic acid;

9-(2,2-Difluoroethoxy)-6-ethyl-10-methoxy-2-oxo-6,7-dihydrobenzo[a]quinolizine-3-carboxylic acid;

(+)-9-(2,2-Difluoroethoxy)-6-ethyl-10-methoxy-2-oxo-6,7-dihydrobenzo[a]quinolizine-3-carboxylic acid;

(−)-9-(2,2-Difluoroethoxy)-6-ethyl-10-methoxy-2-oxo-6,7-dihydrobenzo[a]quinolizine-3-carboxylic acid;

6-Ethyl-10-methoxy-2-oxo-9-(tetrahydropyran-4-ylmethoxy)-6,7-dihydrobenzo[a]quinolizine-3-carboxylic acid;

(+)-6-Ethyl-10-methoxy-2-oxo-9-(tetrahydropyran-4-ylmethoxy)-6,7-dihydrobenzo[a]quinolizine-3-carboxylic acid;

(−)-6-Ethyl-10-methoxy-2-oxo-9-(tetrahydropyran-4-ylmethoxy)-6,7-dihydrobenzo[a]quinolizine-3-carboxylic acid;

6-Ethyl-10-methoxy-2-oxo-9-(2-pyrrolidin-1-ylethoxy)-6,7-dihydrobenzo[a]quinolizine-3-carboxylic acid;

9-(3-Cyanopropoxy)-6-ethyl-10-methoxy-2-oxo-6,7-dihydrobenzo[a]quinolizine-3-carboxylic acid;

6-Ethyl-10-methoxy-9-(2-methylsulfonylethoxy)-2-oxo-6,7-dihydrobenzo[a]quinolizine-3-carboxylic acid;

6-Ethyl-10-methoxy-2-oxo-9-[2-(2-oxopyrrolidin-1-yl)ethoxy]-6,7-dihydrobenzo[a]quinolizine-3-carboxylic acid;

(+)-6-Ethyl-10-methoxy-2-oxo-9-[2-(2-oxopyrrolidin-1-yl)ethoxy]-6,7-dihydrobenzo[a]quinolizine-3-carboxylic acid;

6-Ethyl-9-[2-(methanesulfonamido)ethoxy]-10-methoxy-2-oxo-6,7-dihydrobenzo[a]quinolizine-3-carboxylic acid;

9-[(1-Cyanocyclopropyl)methoxy]-6-ethyl-10-methoxy-2-oxo-6,7-dihydrobenzo[a]quinolizine-3-carboxylic acid;

9-(2-Acetamidoethoxy)-6-ethyl-10-methoxy-2-oxo-6,7-dihydrobenzo[a]quinolizine-3-carboxylic acid;

10-Chloro-9-methoxy-6-methyl-2-oxo-6,7-dihydrobenzo[a]quinolizine-3-carboxylic acid;

9,10-Dimethoxy-6-methyl-2-oxo-6,7-dihydrobenzo[a]quinolizine-3-carboxylic acid;

(+)-9,10-Dimethoxy-6-methyl-2-oxo-6,7-dihydrobenzo[a]quinolizine-3-carboxylic acid;

(−)-9,10-Dimethoxy-6-methyl-2-oxo-6,7-dihydrobenzo[a]quinolizine-3-carboxylic acid;

9,10-Dimethoxy-7-methyl-2-oxo-6,7-dihydrobenzo[a]quinolizine-3-carboxylic acid;

6-Ethyl-9,10-dimethoxy-2-oxo-6,7-dihydrobenzo[a]quinolizine-3-carboxylic acid;

(6R)-(+)-6-Ethyl-9,10-dimethoxy-2-oxo-6,7-dihydrobenzo[a]quinolizine-3-carboxylic acid;

(6S)-(−)-6-Ethyl-9,10-dimethoxy-2-oxo-6,7-dihydrobenzo[a]quinolizine-3-carboxylic acid;

9-Methoxy-6,10-dimethyl-2-oxo-6,7-dihydrobenzo[a]quinolizine-3-carboxylic acid;

9,10-Diethoxy-6-methyl-2-oxo-6,7-dihydrobenzo[a]quinolizine-3-carboxylic acid;

9-Ethoxy-6-methyl-10-hydroxy-2-oxo-6,7-dihydrobenzo[a]quinolizine-3-carboxylic acid;

9,10-Diethoxy-2-oxo-6,7-dihydrobenzo[a]quinolizine-3-carboxylic acid;

2-Oxo-9,10-dipropoxy-6,7-dihydrobenzo[a]quinolizine-3-carboxylic acid;

6-Ethyl-10-methoxy-2-oxo-9-propoxy-6,7-dihydrobenzo[a]quinolizine-3-carboxylic acid;

(+)-6-Ethyl-10-methoxy-2-oxo-9-propoxyl-6,7-dihydrobenzo[a]quinolizine-3-carboxylic acid;

(−)-6-Ethyl-10-methoxy-2-oxo-9-propoxyl-6,7-dihydrobenzo[a]quinolizine-3-carboxylic acid;

8-Chloro-9-methoxy-6-methyl-2-oxo-6,7-dihydrobenzo[a]quinolizine-3-carboxylic acid;

8-Chloro-9,10-dimethoxy-6-methyl-2-oxo-6,7-dihydrobenzo[a]quinolizine-3-carboxylic acid;

10-Benzyloxy-9-methoxy-6-methyl-2-oxo-6,7-dihydrobenzo[a]quinolizine-3-carboxylic acid;

10-Ethoxy-9-methoxy-6-methyl-2-oxo-6,7-dihydrobenzo[a]quinolizine-3-carboxylic acid;

9-Methoxy-6-methyl-2-oxo-10-propoxy-6,7-dihydrobenzo[a]quinolizine-3-carboxylic acid;

6,10-Diethyl-9-methoxy-2-oxo-6,7-dihydrobenzo[a]quinolizine-3-carboxylic acid;

10-Cyclopropyl-6-ethyl-9-methoxy-2-oxo-6,7-dihydrobenzo[a]quinolizine-3-carboxylic acid (compound 19C);

(+)-10-Cyclopropyl-6-ethyl-9-methoxy-2-oxo-6,7-dihydrobenzo[a]quinolizine-3-carboxylic acid;

(−)-10-Cyclopropyl-6-ethyl-9-methoxy-2-oxo-6,7-dihydrobenzo[a]quinolizine-3-carboxylic acid;

9,10-Dimethoxy-2-oxo-6-propyl-6,7-dihydrobenzo[a]quinolizine-3-carboxylic acid;

6-Cyclopropyl-9,10-dimethoxy-2-oxo-6,7-dihydrobenzo[a]quinolizine-3-carboxylic acid;

6-isopropyl-9,10-dimethoxy-2-oxo-6,7-dihydrobenzo[a]quinolizine-3-carboxylic acid;

(+)-6-isopropyl-9,10-dimethoxy-2-oxo-6,7-dihydrobenzo[a]quinolizine-3-carboxylic acid;

(−)-6-isopropyl-9,10-dimethoxy-2-oxo-6,7-dihydrobenzo[a]quinolizine-3-carboxylic acid;

6-isobutyl-9,10-dimethoxy-2-oxo-6,7-dihydrobenzo[a]quinolizine-3-carboxylic acid;

(+)-6-Isobutyl-9,10-dimethoxy-2-oxo-6,7-dihydrobenzo[a]quinolizine-3-carboxylic acid;

(−)-6-Isobutyl-9,10-dimethoxy-2-oxo-6,7-dihydrobenzo[a]quinolizine-3-carboxylic acid;

10-Chloro-6-isobutyl-9-(2-methoxyethoxy)-2-oxo-6,7-dihydrobenzo[a]quinolizine-3-carboxylic acid;

(+)-10-Chloro-6-isobutyl-9-(2-methoxyethoxy)-2-oxo-6,7-dihydrobenzo[a]quinolizine-3-carboxylic acid;

(−)-10-Chloro-6-isobutyl-9-(2-methoxyethoxy)-2-oxo-6,7-dihydrobenzo[a]quinolizine-3-carboxylic acid;

10-Chloro-6-isopropyl-9-(2-methoxyethoxy)-2-oxo-6,7-dihydrobenzo[a]quinolizine-3-carboxylic acid;

(+)-10-Chloro-6-isopropyl-9-(2-methoxyethoxy)-2-oxo-6,7-dihydrobenzo[a]quinolizine-3-carboxylic acid;

(−)-10-Chloro-6-isopropyl-9-(2-methoxyethoxy)-2-oxo-6,7-dihydrobenzo[a]quinolizine-3-carboxylic acid;

10-Fluoro-6-isopropyl-9-(2-methoxyethoxy)-2-oxo-6,7-dihydrobenzo[a]quinolizine-3-carboxylic acid;

11-Chloro-6-isopropyl-9-(2-methoxyethoxy)-2-oxo-6,7-dihydrobenzo[a]quinolizine-3-carboxylic acid;

9,10-Dimethoxy-2-oxo-6-(trifluoromethyl)-6,7-dihydrobenzo[a]quinolizine-3-carboxylic acid;

9-Benzyloxy-6-ethyl-10-methyl-2-oxo-6,7-dihydrobenzo[a]quinolizine-3-carboxylic acid;

(+)-9-Benzyloxy-6-ethyl-10-methyl-2-oxo-6,7-dihydrobenzo[a]quinolizine-3-carboxylic acid;

(−)-9-Benzyloxy-6-ethyl-10-methyl-2-oxo-6,7-dihydrobenzo[a]quinolizine-3-carboxylic acid;

(+)-10-Chloro-9-ethoxy-6-ethyl-2-oxo-6,7-dihydrobenzo[a]quinolizine-3-carboxylic acid;

(−)-10-Chloro-9-ethoxy-6-ethyl-2-oxo-6,7-dihydrobenzo[a]quinolizine-3-carboxylic acid;

10-Chloro-6-isopropyl-9-(3-methoxypropoxy)-2-oxo-6,7-dihydrobenzo[a]quinolizine-3-carboxylic acid;

(+)-10-Chloro-6-isopropyl-9-(3-methoxypropoxy)-2-oxo-6,7-dihydrobenzo[a]quinolizine-3-carboxylic acid;

(−)-10-Chloro-6-isopropyl-9-(3-methoxypropoxy)-2-oxo-6,7-dihydrobenzo[a]quinolizine-3-carboxylic acid;

10-Methoxy-6-isopropyl-9-(3-methoxypropoxy)-2-oxo-6,7-dihydrobenzo[a]quinolizine-3-carboxylic acid;

(+)-10-Methoxy-6-isopropyl-9-(3-methoxypropoxy)-2-oxo-6,7-dihydrobenzo[a]quinolizine-3-carboxylic acid;

(−)-10-Methoxy-6-isopropyl-9-(3-methoxypropoxy)-2-oxo-6,7-dihydrobenzo[a]quinolizine-3-carboxylic acid;

6-Isopropyl-10-methoxy-2-oxo-9-(2,2,2-trifluoroethoxy)-6,7-dihydrobenzo[a]quinolizine-3-carboxylic acid;

(+)-6-Isopropyl-10-methoxy-2-oxo-9-(2,2,2-trifluoroethoxy)-6,7-dihydrobenzo[a]quinolizine-3-carboxylic acid;

(−)-6-Isopropyl-10-methoxy-2-oxo-9-(2,2,2-trifluoroethoxy)-6,7-dihydrobenzo[a]quinolizine-3-carboxylic acid;

(+)-6-Isopropyl-10-methoxy-9-(2-methoxyethoxy)-2-oxo-6,7-dihydrobenzo[a]quinolizine-3-carboxylic acid;

(−)-6-Isopropyl-10-methoxy-9-(2-methoxyethoxy)-2-oxo-6,7-dihydrobenzo[a]quinolizine-3-carboxylic acid;

6-tert-Butyl-10-methoxy-9-(3-methoxypropoxy)-2-oxo-6,7-dihydrobenzo[a]quinolizine-3-carboxylic acid;

(+)-6-tert-Butyl-10-methoxy-9-(3-methoxypropoxy)-2-oxo-6,7-dihydrobenzo[a]quinolizine-3-carboxylic acid;

(−)-6-tert-Butyl-10-methoxy-9-(3-methoxypropoxy)-2-oxo-6,7-dihydrobenzo[a]quinolizine-3-carboxylic acid;

10-Methoxy-9-(2-methoxyethoxy)-6-(1-methylcyclopropyl)-2-oxo-6,7-dihydrobenzo[a]quinolizine-3-carboxylic acid;

(+)-10-Methoxy-9-(2-methoxyethoxy)-6-(1-methylcyclopropyl)-2-oxo-6,7-dihydrobenzo[a]quinolizine-3-carboxylic acid;

(−)-10-Methoxy-9-(2-methoxyethoxy)-6-(1-methylcyclopropyl)-2-oxo-6,7-dihydrobenzo[a]quinolizine-3-carboxylic acid;

11-Chloro-10-fluoro-6-isopropyl-9-(3-methoxypropoxy)-2-oxo-6,7-dihydrobenzo[a]quinolizine-3-carboxylic acid;

(+)-11-Chloro-10-fluoro-6-isopropyl-9-(3-methoxypropoxy)-2-oxo-6,7-dihydrobenzo[a]quinolizine-3-carboxylic acid;

(−)-11-Chloro-10-fluoro-6-isopropyl-9-(3-methoxypropoxy)-2-oxo-6,7-dihydrobenzo[a]quinolizine-3-carboxylic acid;

10-Fluoro-6-isopropyl-9-(3-methoxypropoxy)-2-oxo-6,7-dihydrobenzo[a]quinolizine-3-carboxylic acid;

6-tert-Butyl-9-(2,2-difluoro-3-hydroxy-propoxy)-10-methoxy-2-oxo-6,7-dihydrobenzo[a]quinolizine-3-carboxylic acid;

(+)-9-(2,2-Difluoroethoxy)-6-isopropyl-10-methoxy-2-oxo-6,7-dihydrobenzo[a]quinolizine-3-carboxylic acid;

(−)-9-(2,2-Difluoroethoxy)-6-isopropyl-10-methoxy-2-oxo-6,7-dihydrobenzo[a]quinolizine-3-carboxylic acid;

9-(3-Hydroxy-2,2-dimethyl-propoxy)-6-isopropyl-10-methoxy-2-oxo-6,7-dihydrobenzo[a]quinolizine-3-carboxylic acid;

(+)-9-(3-Hydroxy-2,2-dimethyl-propoxy)-6-isopropyl-10-methoxy-2-oxo-6,7-dihydrobenzo[a]quinolizine-3-carboxylic acid;

(−)-9-(3-Hydroxy-2,2-dimethyl-propoxy)-6-isopropyl-10-methoxy-2-oxo-6,7-dihydrobenzo[a]quinolizine-3-carboxylic acid;

9-(3-Hydroxypropoxy)-6-isopropyl-10-methoxy-2-oxo-6,7-dihydrobenzo[a]quinolizine-3-carboxylic acid;

6-Isopropyl-10-methoxy-9-(4-methoxybutoxy)-2-oxo-6,7-dihydrobenzo[a]quinolizine-3-carboxylic acid;

6-tert-Butyl-9-(3-hydroxy-2,2-dimethyl-propoxy)-10-methoxy-2-oxo-6,7-dihydrobenzo[a]quinolizine-3-carboxylic acid;

(+)-6-tert-Butyl-9-(3-hydroxy-2,2-dimethyl-propoxy)-10-methoxy-2-oxo-6,7-dihydrobenzo[a]quinolizine-3-carboxylic acid;

(−)-6-tert-Butyl-9-(3-hydroxy-2,2-dimethyl-propoxy)-10-methoxy-2-oxo-6,7-dihydrobenzo[a]quinolizine-3-carboxylic acid;

6-tert-Butyl-9-(5-hydroxypentoxy)-10-methoxy-2-oxo-6,7-dihydrobenzo[a]quinolizine-3-carboxylic acid;

(+)-6-tert-Butyl-9-(5-hydroxypentoxy)-10-methoxy-2-oxo-6,7-dihydrobenzo[a]quinolizine-3-carboxylic acid;

(−)-6-tert-Butyl-9-(5-hydroxypentoxy)-10-methoxy-2-oxo-6,7-dihydrobenzo[a]quinolizine-3-carboxylic acid;

6-tert-Butyl-9-(6-hydroxyhexoxy)-10-methoxy-2-oxo-6,7-dihydrobenzo[a]quinolizine-3-carboxylic acid;

(+)-6-tert-Butyl-9-(6-hydroxyhexoxy)-10-methoxy-2-oxo-6,7-dihydrobenzo[a]quinolizine-3-carboxylic acid;

(−)-6-tert-Butyl-9-(6-hydroxyhexoxy)-10-methoxy-2-oxo-6,7-dihydrobenzo[a]quinolizine-3-carboxylic acid;

6-tert-Butyl-9-(4-hydroxybutoxy)-10-methoxy-2-oxo-6,7-dihydrobenzo[a]quinolizine-3-carboxylic acid;

6-tert-Butyl-9-(4-hydroxybut-2-ynoxy)-10-methoxy-2-oxo-6,7-dihydrobenzo[a]quinolizine-3-carboxylic acid;

9-(6-Aminohexoxy)-6-tert-butyl-10-methoxy-2-oxo-6,7-dihydrobenzo[a]quinolizine-3-carboxylic acid;

9-[6-(tert-Butoxycarbonylamino) hexoxy]-6-tert-butyl-10-methoxy-2-oxo-6,7-dihydrobenzo[a]quinolizine-3-carboxylic acid;

(+)-9-[6-(tert-Butoxycarbonylamino)hexoxy]-6-tert-butyl-10-methoxy-2-oxo-6,7-dihydrobenzo[a]quinolizine-3-carboxylic acid;

(−)-9-[6-(tert-Butoxycarbonylamino)hexoxy]-6-tert-butyl-10-methoxy-2-oxo-6,7-dihydrobenzo[a]quinolizine-3-carboxylic acid;

(+)-9-(6-Aminohexoxy)-6-tert-butyl-10-methoxy-2-oxo-6,7-dihydrobenzo[a]quinolizine-3-carboxylic acid hydrochloride;

(−)-9-(6-Aminohexoxy)-6-tert-butyl-10-methoxy-2-oxo-6,7-dihydrobenzo[a]quinolizine-3-carboxylic acid hydrochloride;

9-(8-Aminooctoxy)-6-tert-butyl-10-methoxy-2-oxo-6,7-dihydrobenzo[a]quinolizine-3-carboxylic acid;

9-[8-(tert-Butoxycarbonylamino) octoxy]-6-tert-butyl-10-methoxy-2-oxo-6,7-dihydrobenzo[a]quinolizine-3-carboxylic acid;

(+)-9-[8-(tert-Butoxycarbonylamino)octoxy]-6-tert-butyl-10-methoxy-2-oxo-6,7-dihydrobenzo[a]quinolizine-3-carboxylic acid;

(−)-9-[8-(tert-Butoxycarbonylamino)octoxy]-6-tert-butyl-10-methoxy-2-oxo-6,7-dihydrobenzo[a]quinolizine-3-carboxylic acid;

(+)-9-(8-Aminooctoxy)-6-tert-butyl-10-methoxy-2-oxo-6,7-dihydrobenzo[a]quinolizine-3-carboxylic acid hydrochloride;

(−)-9-(8-Aminooctoxy)-6-tert-butyl-10-methoxy-2-oxo-6,7-dihydrobenzo[a]quinolizine-3-carboxylic acid hydrochloride;

9-[5-(tert-Butoxycarbonylamino)pentoxy]-6-tert-butyl-10-methoxy-2-oxo-6,7-dihydrobenzo[a]quinolizine-3-carboxylic acid;

(+)-9-(5-Aminopentoxy)-6-tert-butyl-10-methoxy-2-oxo-6,7-dihydrobenzo[a]quinolizine-3-carboxylic acid hydrochloride;

(−)-9-(5-Aminopentoxy)-6-tert-butyl-10-methoxy-2-oxo-6,7-dihydrobenzo[a]quinolizine-3-carboxylic acid hydrochloride;

9-(5-Acetamidopentoxy)-6-tert-butyl-10-methoxy-2-oxo-6,7-dihydrobenzo[a]quinolizine-3-carboxylic acid;

6-tert-Butyl-9-[5-(methanesulfonamido)pentoxy]-10-methoxy-2-oxo-6,7-dihydrobenzo[a]quinolizine-3-carboxylic acid;

9-(2-Aminoethoxy)-6-tert-butyl-10-methoxy-2-oxo-6,7-dihydrobenzo[a]quinolizine-3-carboxylic acid;

9-[3-(2-Aminoethoxyl)propoxy]-6-tert-butyl-10-methoxy-2-oxo-6,7-dihydrobenzo[a]quinolizine-3-carboxylic acid;

6-tert-Butyl-9-[3-[2-(ethylamino)ethoxy]propoxy]-10-methoxy-2-oxo-6,7-dihydrobenzo[a]quinolizine-3-carboxylic acid;

6-tert-Butyl-9-(3,3-difluoropropoxy)-10-methoxy-2-oxo-6,7-dihydrobenzo[a]quinolizine-3-carboxylic acid;

6-tert-Butyl-9-(1,1-difluoropropoxy)-10-methoxy-2-oxo-6,7-dihydrobenzo[a]quinolizine-3-carboxylic acid; 6-tert-Butyl-9-(1,1-difluoroallyloxy)-10-methoxy-2-oxo-6,7-dihydrobenzo[a]quinolizine-3-carboxylic acid;

6-tert-Butyl-10-methoxy-9-(3-methylsulfanylpropoxy)-2-oxo-6,7-dihydrobenzo[a]quinolizine-3-carboxylic acid;

(+)-6-tert-Butyl-10-methoxy-9-(3-methylsulfanylpropoxy)-2-oxo-6,7-dihydrobenzo[a]quinolizine-3-carboxylic acid;

(−)-6-tert-Butyl-10-methoxy-9-(3-methylsulfanylpropoxy)-2-oxo-6,7-dihydrobenzo[a]quinolizine-3-carboxylic acid;

6-tert-Butyl-10-methoxy-9-(3-methylsulfonylpropoxy)-2-oxo-6,7-dihydrobenzo[a]quinolizine-3-carboxylic acid;

(+)-6-tert-Butyl-10-methoxy-9-(3-methylsulfonylpropoxy)-2-oxo-6,7-dihydrobenzo[a]quinolizine-3-carboxylic acid;

(−)-6-tert-Butyl-10-methoxy-9-(3-methylsulfonylpropoxy)-2-oxo-6,7-dihydrobenzo[a]quinolizine-3-carboxylic acid;

6-tert-Butyl-10-methoxy-9-(2-morpholinoethoxy)-2-oxo-6,7-dihydrobenzo[a]quinolizine-3-carboxylic acid hydrochloride;

(+)-6-tert-Butyl-10-methoxy-9-(2-morpholinoethoxy)-2-oxo-6,7-dihydrobenzo[a]quinolizine-3-carboxylic acid;

(−)-6-tert-Butyl-10-methoxy-9-(2-morpholinoethoxy)-2-oxo-6,7-dihydrobenzo[a]quinolizine-3-carboxylic acid;

6-tert-Butyl-10-methoxy-9-(3-morpholinopropoxy)-2-oxo-6,7-dihydrobenzo[a]quinolizine-3-carboxylic acid hydrochloride;

(+)-6-tert-Butyl-10-methoxy-9-(3-morpholinopropoxy)-2-oxo-6,7-dihydrobenzo[a]quinolizine-3-carboxylic acid;

(−)-6-tert-Butyl-10-methoxy-9-(3-morpholinopropoxy)-2-oxo-6,7-dihydrobenzo[a]quinolizine-3-carboxylic acid;

6-tert-Butyl-10-methoxy-2-oxo-9-[3-(2-oxopyrrolidin-1-yl)propoxy]-6,7-dihydrobenzo[a]quinolizine-3-carboxylic acid;

6-tert-Butyl-10-methoxy-2-oxo-9-(3-pyrrolidin-1-ylpropoxy)-6,7-dihydrobenzo[a]quinolizine-3-carboxylic acid;

6-Cyclobutyl-10-methoxy-9-(3-methoxypropoxy)-2-oxo-6,7-dihydrobenzo[a]quinolizine-3-carboxylic acid;

9,10-Dimethoxy-2-oxo-6-(2,2,2-trifluoroethyl)-6,7-dihydrobenzo[a]quinolizine-3-carboxylic acid;

10-Methoxy-9-(3-methoxypropoxy)-2-oxo-6-(2,2,2-trifluoroethyl)-6,7-dihydrobenzo[a]quinolizine-3-carboxylic acid;

(+)-10-Methoxy-9-(3-methoxypropoxy)-2-oxo-6-(2,2,2-trifluoroethyl)-6,7-dihydrobenzo[a]quinolizine-3-carboxylic acid;

(−)-10-Methoxy-9-(3-methoxypropoxy)-2-oxo-6-(2,2,2-trifluoroethyl)-6,7-dihydrobenzo[a]quinolizine-3-carboxylic acid;

6-tert-Butyl-10-chloro-9-(3-methoxypropoxy)-2-oxo-6,7-dihydrobenzo[a]quinolizine-3-carboxylic acid;

(+)-6-tert-Butyl-10-chloro-9-(3-methoxypropoxy)-2-oxo-6,7-dihydrobenzo[a]quinolizine-3-carboxylic acid;

(−)-6-tert-Butyl-10-chloro-9-(3-methoxypropoxy)-2-oxo-6,7-dihydrobenzo[a]quinolizine-3-carboxylic acid;

10-Chloro-9-(3-methoxypropoxy)-6-(1-methylcyclopropyl)-2-oxo-6,7-dihydrobenzo[a]quinolizine-3-carboxylic acid (compound 18C);

(+)-10-Chloro-9-(3-methoxypropoxy)-6-(1-methylcyclopropyl)-2-oxo-6,7-dihydrobenzo[a]quinolizine-3-carboxylic acid;

(−)-10-Chloro-9-(3-methoxypropoxy)-6-(1-methylcyclopropyl)-2-oxo-6,7-dihydrobenzo[a]quinolizine-3-carboxylic acid;

10-Methoxy-9-(3-methoxypropoxy)-6-(1-methylcyclopropyl)-2-oxo-6,7-dihydrobenzo[a]quinolizine-3-carboxylic acid;

(+)-10-Methoxy-9-(3-methoxypropoxy)-6-(1-methylcyclopropyl)-2-oxo-6,7-dihydrobenzo[a]quinolizine-3-carboxylic acid;

(−)-10-Methoxy-9-(3-methoxypropoxy)-6-(1-methylcyclopropyl)-2-oxo-6,7-dihydrobenzo[a]quinolizine-3-carboxylic acid;

6-Benzyl-9,10-dimethoxy-2-oxo-6,7-dihydrobenzo[a]quinolizine-3-carboxylic acid;

10-Chloro-6-ethyl-9-(2-methoxyethoxy)-7-methyl-2-oxo-6,7-dihydrobenzo[a]quinolizine-3-carboxylic acid;

(6R*,7S*)-10-Chloro-6-ethyl-9-(2-methoxyethoxy)-7-methyl-2-oxo-6,7-dihydrobenzo[a]quinolizine-3-carboxylic acid;

(6R*,7R*)-10-Chloro-6-ethyl-9-(2-methoxyethoxy)-7-methyl-2-oxo-6,7-dihydrobenzo[a]quinolizine-3-carboxylic acid;

10,11-Difluoro-6-isopropyl-9-(3-methoxypropoxy)-2-oxo-6,7-dihydrobenzo[a]quinolizine-3-carboxylic acid;

(+)-10,11-Difluoro-6-isopropyl-9-(3-methoxypropoxy)-2-oxo-6,7-dihydrobenzo[a]quinolizine-3-carboxylic acid;

(−)-10,11-Difluoro-6-isopropyl-9-(3-methoxypropoxy)-2-oxo-6,7-dihydrobenzo[a]quinolizine-3-carboxylic acid;

6-tert-Butyl-10-methoxy-2-oxo-9-(3-pyrazol-1-ylpropoxy)-6,7-dihydrobenzo[a]quinolizine-3-carboxylic acid;

6-tert-Butyl-10-methoxy-2-oxo-9-[3-(1,2,4-triazol-1-yl)propoxy]-6,7-dihydrobenzo[a]quinolizine-3-carboxylic acid;

6-tert-Butyl-9-(3-carboxypropoxy)-10-methoxy-2-oxo-6,7-dihydrobenzo[a]quinolizine-3-carboxylic acid;

9-Bromo-6-methyl-2-oxo-6,7-dihydrobenzo[a]quinolizine-3-carboxylic acid;

11-Bromo-6-methyl-2-oxo-6,7-dihydrobenzo[a]quinolizine-3-carboxylic acid;

(+)-9-Bromo-6-methyl-2-oxo-6,7-dihydrobenzo[a]quinolizine-3-carboxylic acid;

9-(4-tert-Butoxycarbonylpiperazin-1-yl)-6-methyl-2-oxo-6,7-dihydrobenzo[a]quinolizine-3-carboxylic acid;

9-[Benzyl(methyl)amino]-6-methyl-2-oxo-6,7-dihydrobenzo[a]quinolizine-3-carboxylic acid;

6-Methyl-11-(methylamino)-2-oxo-6,7-dihydrobenzo[a]quinolizine-3-carboxylic acid;

6-Ethyl-10-methoxy-2-oxo-9-propyl-6,7-dihydrobenzo[a]quinolizine-3-carboxylic acid;

10-Methoxy-6-methyl-2-oxo-6,7-dihydrobenzo[a]quinolizine-3-carboxylic acid;

9-Bromo-6-ethyl-10-methoxy-2-oxo-6,7-dihydrobenzo[a]quinolizine-3-carboxylic acid;

9-Cyano-6-ethyl-10-methoxy-2-oxo-6,7-dihydrobenzo[a]quinolizine-3-carboxylic acid;

8-Bromo-11-ethoxy-6-ethyl-2-oxo-6,7-dihydrobenzo[a]quinolizine-3-carboxylic acid;

8-Cyano-11-ethoxy-6-ethyl-2-oxo-6,7-dihydrobenzo[a]quinolizine-3-carboxylic acid;

6-Ethyl-9,10-dimethyl-2-oxo-6,7-dihydrobenzo[a]quinolizine-3-carboxylic acid; and

6-Ethyl-8,9-dimethyl-2-oxo-6,7-dihydrobenzo[a]quinolizine-3-carboxylic acid;

or a pharmaceutically acceptable salt thereof

In some embodiments, the compound of Formula (II) is selected from:

9-benzyloxy-10-methoxy-6-methyl-2-oxo-6,7-dihydrobenzo[a]quinolizine-3-carboxylic acid;

9,10-Diethoxy-6-ethyl-2-oxo-6,7-dihydrobenzo[a]quinolizine-3-carboxylic acid;

(+)-6-Ethyl-10-methoxy-2-oxo-9-(2,2,2-trifluoroethoxy)-6,7-dihydrobenzo[a]quinolizine-3-carboxylic acid;

9-Butoxy-6-ethyl-10-methoxy-2-oxo-6,7-dihydrobenzo[a]quinolizine-3-carboxylic acid;

6-Ethyl-9-(3-hydroxy-2,2-dimethyl-propoxy)-10-methoxy-2-oxo-6,7-dihydrobenzo[a]quinolizine-3-carboxylic acid;

6-Ethyl-10-methoxy-9-(2-methoxyethoxy)-2-oxo-6,7-dihydrobenzo[a]quinolizine-3-carboxylic acid;

9-(Cyclopropylmethoxy)-6-ethyl-10-methoxy-2-oxo-6,7-dihydrobenzo[a]quinolizine-3-carboxylic acid;

6-Ethyl-9-isobutoxy-10-methoxy-2-oxo-6,7-dihydrobenzo[a]quinolizine-3-carboxylic acid;

9-Ethoxy-6-ethyl-10-methoxy-2-oxo-6,7-dihydrobenzo[a]quinolizine-3-carboxylic acid;

6-Ethyl-10-methoxy-9-(3-methoxypropoxy)-2-oxo-6,7-dihydrobenzo[a]quinolizine-3-carboxylic acid;

9-(2-Ethoxyethoxy)-6-ethyl-10-methoxy-2-oxo-6,7-dihydrobenzo[a]quinolizine-3-carboxylic acid;

6-Ethyl-10-methoxy-2-oxo-9-(tetrahydropyran-4-ylmethoxy)-6,7-dihydrobenzo[a]quinolizine-3-carboxylic acid;

6-Ethyl-9,10-dimethoxy-2-oxo-6,7-dihydrobenzo[a]quinolizine-3-carboxylic acid;

(6R)-(+)-6-Ethyl-9,10-dimethoxy-2-oxo-6,7-dihydrobenzo[a]quinolizine-3-carboxylic acid;

6-Ethyl-10-methoxy-2-oxo-9-propoxy-6,7-dihydrobenzo[a]quinolizine-3-carboxylic acid;

6,10-Diethyl-9-methoxy-2-oxo-6,7-dihydrobenzo[a]quinolizine-3-carboxylic acid;

10-Cyclopropyl-6-ethyl-9-methoxy-2-oxo-6,7-dihydrobenzo[a]quinolizine-3-carboxylic acid;

6-Isopropyl-9,10-dimethoxy-2-oxo-6,7-dihydrobenzo[a]quinolizine-3-carboxylic acid;

6-Isobutyl-9,10-dimethoxy-2-oxo-6,7-dihydrobenzo[a]quinolizine-3-carboxylic acid;

10-Chloro-6-isobutyl-9-(2-methoxyethoxy)-2-oxo-6,7-dihydrobenzo[a]quinolizine-3-carboxylic acid;

10-Chloro-6-isopropyl-9-(2-methoxyethoxy)-2-oxo-6,7-dihydrobenzo[a]quinolizine-3-carboxylic acid;

10-Fluoro-6-isopropyl-9-(2-methoxyethoxy)-2-oxo-6,7-dihydrobenzo[a]quinolizine-3-carboxylic acid;

9-Benzyloxy-6-ethyl-10-methyl-2-oxo-6,7-dihydrobenzo[a]quinolizine-3-carboxylic acid;

10-Chloro-6-isopropyl-9-(3-methoxypropoxy)-2-oxo-6,7-dihydrobenzo[a]quinolizine-3-carboxylic acid;

10-Methoxy-6-isopropyl-9-(3-methoxypropoxy)-2-oxo-6,7-dihydrobenzo[a]quinolizine-3-carboxylic acid;

(+)-10-Methoxy-6-isopropyl-9-(3-methoxypropoxy)-2-oxo-6,7-dihydrobenzo[a]quinolizine-3-carboxylic acid;

6-Isopropyl-10-methoxy-2-oxo-9-(2,2,2-trifluoroethoxy)-6,7-dihydrobenzo[a]quinolizine-3-carboxylic acid;

(+)-6-Isopropyl-10-methoxy-9-(2-methoxyethoxy)-2-oxo-6,7-dihydrobenzo[a]quinolizine-3-carboxylic acid;

6-tert-Butyl-10-methoxy-9-(3-methoxypropoxy)-2-oxo-6,7-dihydrobenzo[a]quinolizine-3-carboxylic acid;

(+)-6-tert-Butyl-10-methoxy-9-(3-methoxypropoxy)-2-oxo-6,7-dihydrobenzo[a]quinolizine-3-carboxylic acid;

10-Methoxy-9-(2-methoxyethoxy)-6-(1-methylcyclopropyl)-2-oxo-6,7-dihydrobenzo[a]quinolizine-3-carboxylic acid;

(+)-10-Methoxy-9-(2-methoxyethoxy)-6-(1-methylcyclopropyl)-2-oxo-6,7-dihydrobenzo[a]quinolizine-3-carboxylic acid;

11-Chloro-10-fluoro-6-isopropyl-9-(3-methoxypropoxy)-2-oxo-6,7-dihydrobenzo[a] quinolizine-3-carboxylic acid;

10-Fluoro-6-isopropyl-9-(3-methoxypropoxy)-2-oxo-6,7-dihydrobenzo[a]quinolizine-3-carboxylic acid;

6-tert-Butyl-9-(2,2-difluoro-3-hydroxy-propoxy)-10-methoxy-2-oxo-6,7-dihydrobenzo[a]quinolizine-3-carboxylic acid;

(+)-9-(2,2-Difluoroethoxy)-6-isopropyl-10-methoxy-2-oxo-6,7-dihydrobenzo[a]quinolizine-3-carboxylic acid;

9-(3-Hydroxy-2,2-dimethyl-propoxy)-6-isopropyl-10-methoxy-2-oxo-6,7-dihydrobenzo[a]quinolizine-3-carboxylic acid;

9-(3-Hydroxypropoxy)-6-isopropyl-10-methoxy-2-oxo-6,7-dihydrobenzo[a]quinolizine-3-carboxylic acid;

6-Isopropyl-10-methoxy-9-(4-methoxybutoxy)-2-oxo-6,7-dihydrobenzo[a]quinolizine-3-carboxylic acid;

6-tert-Butyl-9-(3-hydroxy-2,2-dimethyl-propoxy)-10-methoxy-2-oxo-6,7-dihydrobenzo[a]quinolizine-3-carboxylic acid;

6-tert-Butyl-9-(5-hydroxypentoxy)-10-methoxy-2-oxo-6,7-dihydrobenzo[a]quinolizine-3-carboxylic acid;

6-tert-Butyl-9-(6-hydroxyhexoxy)-10-methoxy-2-oxo-6,7-dihydrobenzo[a]quinolizine-3-carboxylic acid;

6-tert-Butyl-9-(4-hydroxybutoxy)-10-methoxy-2-oxo-6,7-dihydrobenzo[a]quinolizine-3-carboxylic acid;

9-(6-Aminohexoxy)-6-tert-butyl-10-methoxy-2-oxo-6,7-dihydrobenzo[a]quinolizine-3-carboxylic acid;

9-[8-(tert-Butoxycarbonylamino)octoxy]-6-tert-butyl-10-methoxy-2-oxo-6,7-dihydrobenzo[a]quinolizine-3-carboxylic acid;

9-[5-(tert-Butoxycarbonylamino)pentoxy]-6-tert-butyl-10-methoxy-2-oxo-6,7-dihydrobenzo[a]quinolizine-3-carboxylic acid;

9-(5-Acetamidopentoxy)-6-tert-butyl-10-methoxy-2-oxo-6,7-dihydrobenzo[a]quinolizine-3-carboxylic acid;

6-tert-Butyl-9-[5-(methanesulfonamido)pentoxy]-10-methoxy-2-oxo-6,7-dihydrobenzo[a]quinolizine-3-carboxylic acid;

6-tert-Butyl-9-[3-[2-(ethylamino)ethoxy]propoxy]-10-methoxy-2-oxo-6,7-dihydrobenzo[a]quinolizine-3-carboxylic acid;

6-tert-Butyl-9-(3,3-difluoropropoxy)-10-methoxy-2-oxo-6,7-dihydrobenzo[a]quinolizine-3-carboxylic acid;

6-tert-Butyl-9-(1,1-difluoropropoxy)-10-methoxy-2-oxo-6,7-dihydrobenzo[a]quinolizine-3-carboxylic acid;

6-tert-Butyl-10-methoxy-9-(3-methylsulfanylpropoxy)-2-oxo-6,7-dihydrobenzo[a]quinolizine-3-carboxylic acid;

6-tert-Butyl-10-methoxy-9-(3-methylsulfonylpropoxy)-2-oxo-6,7-dihydrobenzo[a]quinolizine-3-carboxylic acid;

6-tert-Butyl-10-methoxy-9-(2-morpholinoethoxy)-2-oxo-6,7-dihydrobenzo[a]quinolizine-3-carboxylic acid hydrochloride;

6-tert-Butyl-10-methoxy-9-(3-morpholinopropoxy)-2-oxo-6,7-dihydrobenzo[a]quinolizine-3-carboxylic acid hydrochloride;

6-Cyclobutyl-10-methoxy-9-(3-methoxypropoxy)-2-oxo-6,7-dihydrobenzo[a]quinolizine-3-carboxylic acid;

10-Methoxy-9-(3-methoxypropoxy)-2-oxo-6-(2,2,2-trifluoroethyl)-6,7-dihydrobenzo[a]quinolizine-3-carboxylic acid;

6-tert-Butyl-10-chloro-9-(3-methoxypropoxy)-2-oxo-6,7-dihydrobenzo[a]quinolizine-3-carboxylic acid;

10-Chloro-9-(3-methoxypropoxy)-6-(1-methylcyclopropyl)-2-oxo-6,7-dihydrobenzo[a]quinolizine-3-carboxylic acid;

10-Methoxy-9-(3-methoxypropoxy)-6-(1-methylcyclopropyl)-2-oxo-6,7-dihydrobenzo[a]quinolizine-3-carboxylic acid;

(6R*,7S*)-10-Chloro-6-ethyl-9-(2-methoxyethoxy)-7-methyl-2-6,7-dihydrobenzo[a]quinolizine-3-carboxylic acid; and

10,11-Difluoro-6-isopropyl-9-(3-methoxypropoxy)-2-oxo-6,7-dihydrobenzo[a]quinolizine-3-carboxylic acid,

or a pharmaceutically acceptable salt thereof.

In some embodiments, the compound of Formula (II) is 6-isopropyl-10-methoxy-9-(3-methoxypropoxy)-2-oxo-6,7-dihydro-2H-pyrido[2,1-a]isoquinoline-3-carboxylic acid, or a pharmaceutically acceptable salt thereof.

In some embodiments, the compound of Formula (II) is (S)-6-isopropyl-10-methoxy-9-(3-methoxypropoxy)-2-oxo-6,7-dihydro-2H-pyrido[2,1-a]isoquinoline-3-carboxylic acid, or a pharmaceutically acceptable salt thereof.

In some embodiments, the compound of Formula (II) is (R)-6-isopropyl-10-methoxy-9-(3-methoxypropoxy)-2-oxo-6,7-dihydro-2H-pyrido[2,1-a]isoquinoline-3-carboxylic acid, or a pharmaceutically acceptable salt thereof.

In some embodiments, the compound of Formula (II) is:

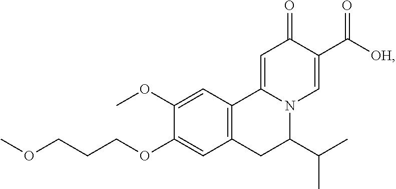 [SMILES: COCCCOc1cc2c(cc1OC)-c1cc(=O)c(C(=O)O)cn1C(C(C)C)C2]

or a pharmaceutically acceptable salt thereof.

In some embodiments, the compound of Formula (II) is:

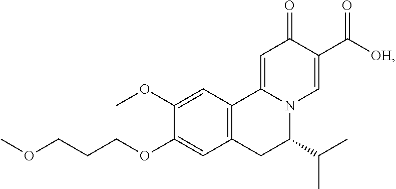 [SMILES: COCCCOc1cc2c(cc1OC)-c1cc(=O)c(C(=O)O)cn1[C@H](C(C)C)C2]

or a pharmaceutically acceptable salt thereof.

In some embodiments, the compound of Formula (II) is:

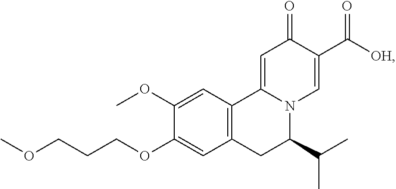 [SMILES: COCCCOc1cc2c(cc1OC)-c1cc(=O)c(C(=O)O)cn1[C@@H](C(C)C)C2]

or a pharmaceutically acceptable salt thereof.

In some embodiments, the compound of Formula (II) is 10-Chloro-9-(3-methoxypropoxy)-6-(1-methylcyclopropyl)-2-oxo-6,7-dihydrobenzo[a]quinolizine-3-carboxylic acid:

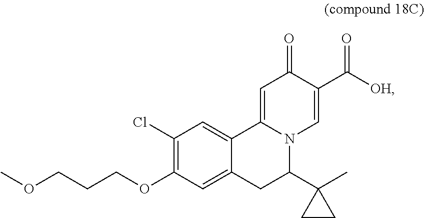 [SMILES: COCCCOc1cc2c(cc1Cl)-c1cc(=O)c(C(=O)O)cn1C(C1(C)CC1)C2]

or a pharmaceutically acceptable salt thereof.

In some embodiments, the compound of Formula (II) is 10-Cyclopropyl-6-ethyl-9-methoxy-2-oxo-6,7-dihydrobenzo[a]quinolizine-3-carboxylic acid:

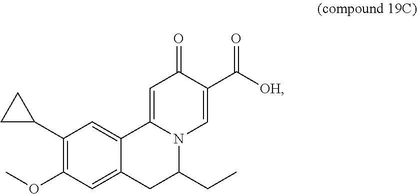 [SMILES: CCC1Cc2cc(OC)c(C3CC3)cc2-c2cc(=O)c(C(=O)O)cn21]

or a pharmaceutically acceptable salt thereof.

In some embodiments, the compound of Formula (II) is any one of compounds described in WO2015113990A1, US20150210682A1, US20160296515A1, U.S. Pat. Nos. 9,458,153B2, 9,949,966B2, WO2015173164A1, US20170057952A1, U.S. Pat. No. 9,920,049B2, US20160122344A1, U.S. Pat. No. 9,637,485B2, WO2016071215A1, WO2016107832A1, US20170298067A1, WO2016128335A1, US20170342069A1, WO2016177655A1, US20160326167A1, U.S. Pat. No. 9,845,322B2, WO2017017042A1, US20180127416A1, WO2017216391A1, WO2017216390A1, and WO2018154466, the content of which is incorporated herein by reference in their entirety.

As used herein, the term “pharmaceutically acceptable salt” refers to a salt that is formed between an acid and a basic group of the compound, such as an amino functional group, or between a base and an acidic group of the compound, such as a carboxyl functional group. In some embodiments, the compound is a pharmaceutically acceptable acid addition salt. In some embodiments, acids commonly employed to form pharmaceutically acceptable salts of the therapeutic compounds described herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, P-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the therapeutic compounds described herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Methods of Making

Compounds of any one of the Formulae disclosed herein, including salts thereof, can be prepared using known organic synthesis techniques and can be synthesized according to any of numerous possible synthetic routes. For example, the compounds described herein can be prepared using methods and procedures similar to those described in Donnelly, A. et al, The Design, Synthesis, and Evaluation of Coumarin Ring Derivatives of the Novobiocin Scaffold that Exhibit Antiproliferative Activity, Journal of Organic Chemistry 2008, 73, 8901-8920, which is incorporated herein by reference in its entirety. A person skilled in the art knows how to select and implement appropriate synthetic protocols, and appreciates that a broad repertoire of synthetic organic reactions is available to be potentially employed in synthesizing compounds provided herein.

Suitable synthetic methods of starting materials, intermediates and products can be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

Methods of Use

### Modulation of Telomerase RNA Component (TERC)

Telomerase has been a therapeutic target of great interest for over two decades, based on its activity in numerous cancers. The telomerase RNA component (TERC) contains a box H/ACA domain at its 3′ end, a motif that is functionally separable from the template domain and dispensable for telomerase activity in vitro. In vivo, the H/ACA motif is bound by a heterotrimer of dyskerin, NOP10, and NHP2 which stabilize TERC, and also by TCAB1, which is responsible for localizing the telomerase complex to Cajal bodies (Venteicher, A. S. et al. A human telomerase holoenzyme protein required for Cajal body localization and telomere synthesis. Science 323, 644-8 (2009)). Disruption of any of these interactions can also compromise telomere maintenance and cause telomere disease (Mitchell, J. R., Wood, E. & Collins, K. A telomerase component is defective in the human disease dyskeratosis congenita. Nature 402, 551-5 (1999); Vulliamy, T. et al. Mutations in the telomerase component NHP2 cause the premature ageing syndrome dyskeratosis congenita. Proceedings of the National Academy of Sciences of the United States of America 105, 8073-8 (2008); Walne, A. J. et al. Genetic heterogeneity in autosomal recessive dyskeratosis congenita with one subtype due to mutations in the telomerase-associated protein NOP10. Human molecular genetics 16, 1619-29 (2007)). The H/ACA motif serve as guides for pseudouridylation of other RNAs by dyskerin (Kiss, T., Fayet-Lebaron, E. & Jady, B. E. Box H/ACA small ribonucleoproteins. Molecular cell 37, 597-606 (2010)).

Increasing telomerase activity can be beneficial in several degenerative and age-related disorders. Conversely, inhibiting telomerase activity would be of significant utility for the treatment of cancer and disorders in which hyper-proliferative cells depend on telomerase for self-renewal.

### Modulation of Poly(A) Specific Ribonuclease (PARN)

PARN is known as a 3′-5′ exoribonuclease responsible for degradation of the poly(A) tails of eukaryotic mRNAs, which is a rate-limiting step in mRNA turnover (Korner, C. G. & Wahle, E. Poly(A) tail shortening by a mammalian poly(A)-specific 3′-exoribonuclease. The Journal of biological chemistry 272, 10448-56 (1997)). PARN is stimulated by presence of a m7G-cap, and requires a minimal substrate of adenosine di- or tri-nucleotides—in other words, oligo(A) rather than strictly poly(A). PARN is a widely-expressed cap-dependent, poly(A) deadenylase with a canonical role in regulating global mRNA levels during development, and additional, more specialized functions including end-trimming of the Dicer-independent microRNA (miR)-451 and deadenylation of small nucleolar (sno)RNAs. PARN loss-of-function mutations are implicated in idiopathic pulmonary fibrosis and dyskeratosis congenita. The disclosure provides methods and agents that modulate the level or activity of human PARN. The nucleotide sequence of human PARN is NM_002582 and the amino acid sequence of PARN is O95453 (Tablet). Variants of the nucleotide sequence and the amino acid sequence are also shown in Table 1.

TABLE 1
Accession numbers for genes, RNA and proteins

| | | Nucleotide | Protein ID(s) |
|---|---|---|---|
| | | sequence(s) and | and variants |
| | | variants therein | therein |
| | | (RefSeq unless | (Uniprot unless |
| | | othewise | otherwise |
| Gene | Ensembl Gene ID | indicated) | indicated) |
| TERC | ENSG00000270141 | NR_001566 | N/A |
| PARN | ENSG00000140694 | NM_002582 | O96453 |
| | | NM_001242992 | |
| | | NM_1134477 | |
| TRF4-2 | ENSG00000121274 | NM_001040284 | Q8NDF8 |
| A.k.a. | | | |
| PAPD5 | | | |
| | | NM_001040285 | H3BQM0 |
| | | FR872509.1 | CCB84642.1 |
| | | (GenBank) | (GenBank) |

### PAP Associated Domain Containing 5 (PAPD5)

PAPD5, also known as Topoisomerase-Related Function Protein 4-2 (TRF4-2), is one of the seven members of the family of noncanonical poly(A) polymerases in human cells. TRF4-2 has been shown to act as a polyadenylase on abnormal pre-ribosomal RNAs in vivo in a manner analogous to degradation-mediating polyadenylation by the non-canonical poly(A) polymerase Trf4p in yeast. PAPD5 is also involved in the uridylation-dependent degradation of histone mRNAs.

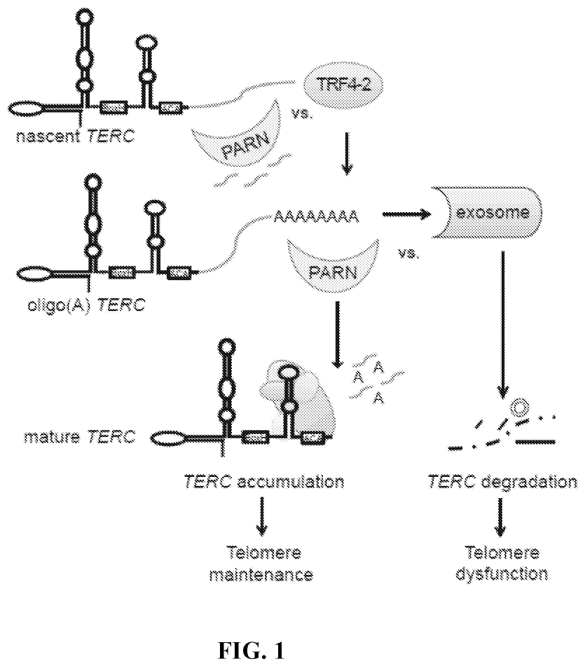

Both PARN and PAPD5 are involved in the 3′-end maturation of the telomerase RNA component (TERC). Patient cells, fibroblast cells as well as converted fibroblasts (iPS cells) in which PARN is disrupted show decreased levels of TERC which can be restored by decreasing levels or activities of PAPD5. Deep sequencing of TERC RNA 3′ termini or ends, reveals that PARN and PAPD5 are critically important for processing of post-transcriptionally acquired oligo(A) tails that target nuclear RNAs for degradation. Diminished TERC levels and the increased oligo(A) forms of TERC are normalized by restoring PARN or inhibiting PAPD5. The disclosure reveals PARN and PAPD5 as important players in the regulation and biogenesis of TERC (FIG. 1). FIG. 1 shows 3′ ends of nascent TERC RNA are subject to PAPD5-mediated oligo-adenylation, which targets transcripts for degradation by the exosome. PARN counteracts the degradation pathway by removing oligo(A) tails and/or trimming genomically-encoded bases (green) of nascent TERC to yield a mature 3′ end. Mature TERC is protected from further oligo-adenylation and exonucleolytic processing, possibly by the dyskerin/NOP10NHP2/GAR1 complex, and assembles into the telomerase holoenzyme to maintain telomeres. PARN deficiency tips the balance in favor of degradation, leading to reduced TERC levels and telomere dysfunction. Thus, the disclosure also provides compounds and methods that modulate the level or activity of human PAPD5. The nucleotide sequence of human PAPD5 used is FR872509.1, and the amino acid sequence is CCB84642.1 (Table 1). Variants of the nucleotide sequence and the amino acid sequence are also shown in Table 1. The amino acid sequence of PAPD5 used is shown below:

PAPD5 (TRF4-2) (CCB84642.1)
(SEQ ID NO: 1)
MYRSGERLLG SHALPAEQRD FLPLETTNNN NNHHQPGAWA
RRAGSSASSP PSASSSPHPS
AAVPAADPAD SASGSSNKRK RDNKASTYGL NYSLLQPSGG
RAAGGGRADG GGVVYSGTPW
KRRNYNQGVV GLHEEISDFY EYMSPRPEEE KMRMEVVNRI
ESVIKELWPS ADVQIFGSFK
TGLYLPTSDI DLVVFGKWEN LPLWTLEEAL RKHKVADEDS
VKVLDKATVP IIKLTDSFTE
VKVDISFNVQ NGVRAADLIK DFTKKYPVLP YLVLVLKQFL
LQRDLNEVFT GGIGSYSLFL MAVSFLQLHP REDACIPNTN
YGVLLIEFFE LYGRHFNYLK TGIRIKDGGS YVAKDEVQKN
MLDGYRPSML YIEDPLQPGN DVGRSSYGAM QVKQAFDYAY
VVLSHAVSPI AKYYPNNETE
SILGRIIRVT DEVATYRDWI SKQWGLKNRP EPSCNGNGVT
LIVDTQQLDK CNNNLSEENE
ALGKCRSKTS ESLSKHSSNS SSGPVSSSSA TQSSSSDVDS
DATPCKTPKQ LLCRPSTGNR
VGSQDVSLES SQAVGKMQST QTTNTSNSTN KSQHGSARLF
RSSSKGFQGT TQTSHGSLMT
NKQHQGKSNN QYYHGKKRKH KRDAPLSDLC R

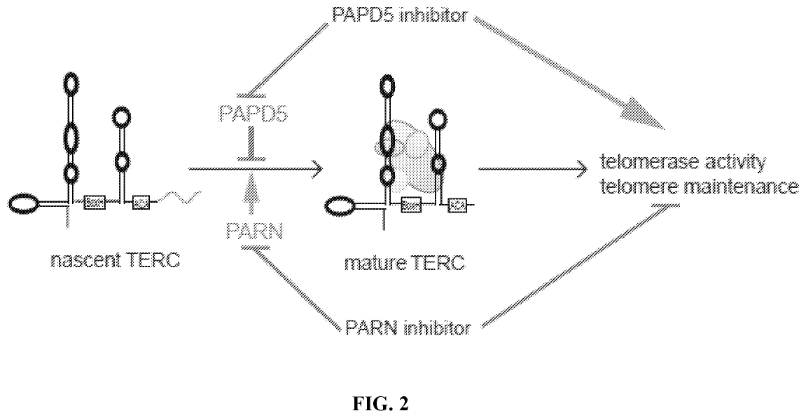

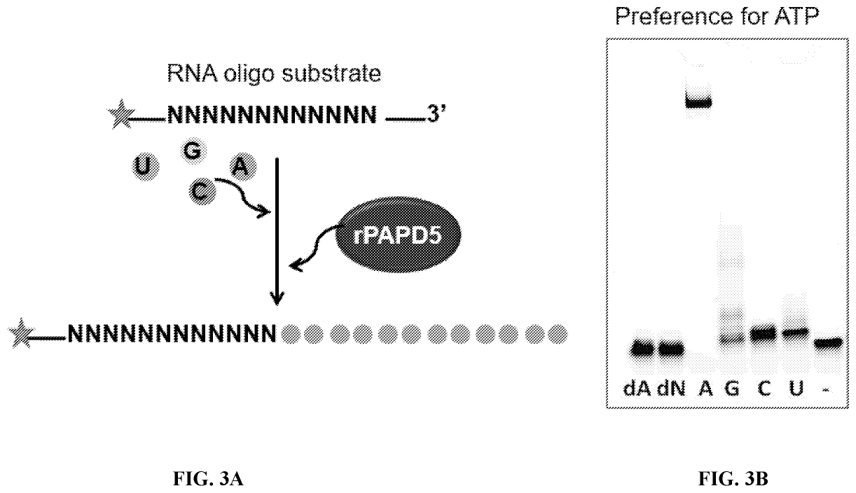

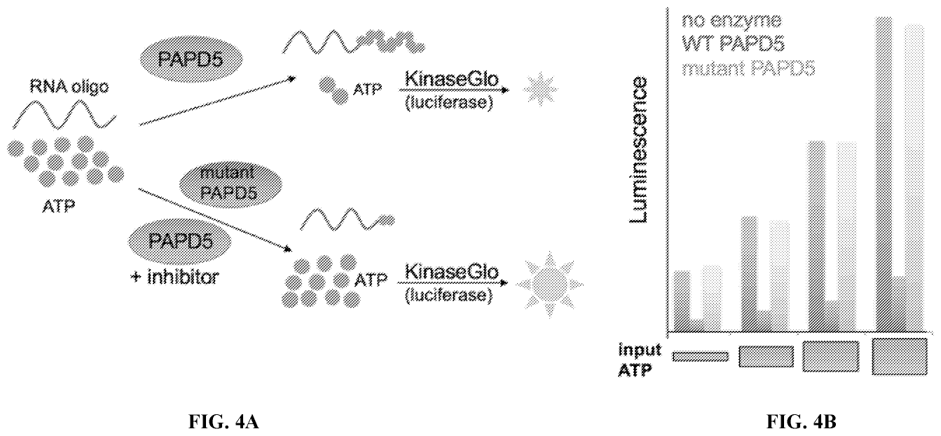

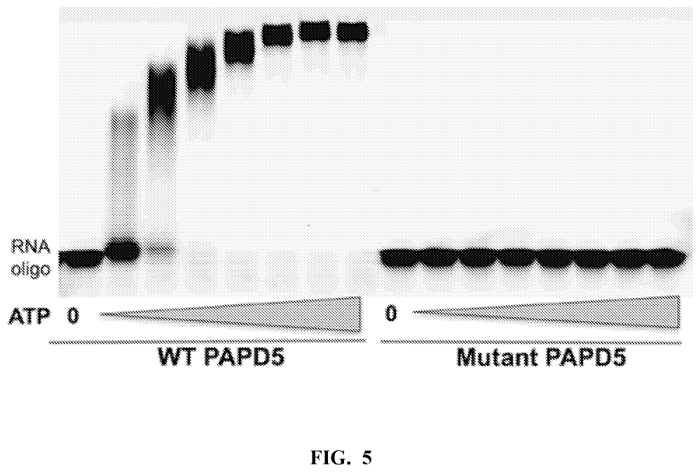

FIG. 2 is a diagram demonstrating the reciprocal regulation of TERC levels by PAPD5 and PARN, and the potential for therapeutic manipulation of telomerase in degenerative or malignant disorders. As shown in FIG. 2, a PAPD5 inhibitor can inhibit PAPD5-mediated oligo-adenylation, which targets nascent TERC RNA for degradation by the exosome, thus increases the level or activity of TERC. In contrast, as PARN counteracts the degradation pathway by removing oligo(A) tails and/or trimming genomically-encoded bases of nascent TERC to yield a mature 3′ end, PARN inhibitor will decrease the level or activity of TERC. In addition, increasing the level or activity of PARN can increase the level or activity of TERC, and increasing the level or activity of PAPD5 can decrease the level or activity of TERC.

In one aspect, the present disclosure provides compounds and associated methods of modulating TERC levels in cells. The cells can be, e.g., primary human cells, stem cells, induced pluripotent cells, fibroblasts, etc. In some embodiments, the cells are within a subject (e.g., a human subject). Therefore, the present disclosure provides methods modulating TERC levels in cells in vivo. In some embodiments, the cells can be isolated from a sample obtained from the subject, e.g., the cells can be derived from any part of the body including, but not limited to, skin, blood, and bone marrow. The cells can also be cultured in vitro using routine methods with commercially available cell reagents (e.g., cell culture media). In some embodiments, the cells are obtained from a subject, having a telomere disease, being at risk of developing a telomere disease, or being suspected of having a telomere disease. In some embodiments, the subject has no overt symptoms.

The level or activity of TERC can be determined by various means, e.g., by determining the size of telomere in the cell, by determining the stability of TERC, by determining the amount of RNA, by measuring the activity of telomerase function, and/or by measuring oligo-adenylated (oligo(A)) forms of TERC. TERC stability can be assessed, e.g., by measuring the TERC decay rates. Oligo-adenylated (oligo(A)) forms of TERC can be measured, e.g., using rapid amplification of cDNA ends (RACE) coupled with targeted deep sequencing (e.g., at the TERC 3′ end) to detect oligo-adenylated (oligo(A)) forms of TERC. The size of a telomere can be measured, e.g., using Flow-fluorescent in-situ hybridization (Flow-FISH) technique.

In some embodiments, the modulation of endogenous TERC is performed. Such methods can include, e.g., altering telomerase activity, e.g., increasing or decreasing telomerase activity. The methods can involve reducing RNA expression in cells, e.g., non-coding RNA in TERC. Telomerase activity can be, e.g., regulated by modulating TERC levels by contacting cells with test compounds known to modulate protein synthesis. The methods may involve targeting post-processing activity of the endogenous TERC locus. These methods involve manipulating TERC including identifying subjects with genetic mutation (e.g., mutation in PARN), isolating cells (e.g., fibroblast), and treating cells with agents that modulate TERC levels.

The present disclosure shows that TERC levels are modulated at the post-transcriptional level. Thus, in one aspect, methods of modulating the level or activity of TERC involve modulating the level or activity of PARN and PAPD5.

In some embodiments, the methods involve an agent that modulates the level or activity of PARN, thereby altering the level or activity of TERC. In some cases, the agent increases the level or activity of PARN. Alternatively, the agent decreases the level or activity of PARN. In some embodiments, the methods involve an agent that modulates the level or activity of PAPD5, thereby altering the level or activity of TERC. In some embodiments, the agent increases the level or activity of PAPD5. Alternatively, the agent decreases the level or activity of PAPD5 (e.g., PAPD5 inhibitors). In some embodiments, the agent is any one of compounds described herein.

Accordingly, the present application provides compounds that modulate TERC levels and are thus useful in treating a broad array of telomere diseases or disorders associated with telomerase dysfunction, e.g., dyskeratosis congenita, aplastic anemia, pulmonary fibrosis, idiopathic pulmonary fibrosis, hematological disorder, hepatic disease (e.g., chronic liver disease), and cancer, e.g., hematological cancer and hepatocarcinoma, etc.

In some embodiments, it was surprisingly discovered that in order to successfully treat a telomere disease, a therapeutic agent has to be a selective inhibitor of PAPD5. In other words, a successful therapeutic agent has to inhibit PAPD5 while not substantially inhibiting PARN and/or other polynucleotide polymerases. In some embodiments, a PAPD5 inhibitor that is not selective to PAPD5 and concurrently inhibits other polymerases, may not be useful in treating telomere diseases; that is, the fact that a compound is a PAPD5 inhibitor (e.g., non-selective inhibitor) is not indicative of its usefulness in prevention and treatment of telomere diseases. The selectivity to PAPD5 as opposed to other polymerases is required for potency. In some embodiments, the compounds of the present application are selective and specific inhibitors of PAPD5 and do not substantially inhibit PARN or other polymerases.

Telomere Diseases

Telomere diseases or disorders associated with telomerase dysfunction are typically associated with changes in the size of telomere. Many proteins and RNA components are involved in the telomere regulatory pathway, including TERC, PARN and PAPD5 (also known as TRF4-2, TENT4B, TUT3, GLD4). FIGS. 1 and 2 show how these proteins or RNA components work in the regulatory pathway and how they are related to telomere diseases.

Among these telomere diseases is dyskeratosis congenita (DC), which is a rare, progressive bone marrow failure syndrome characterized by the triad of reticulated skin hyperpigmentation, nail dystrophy, and oral leukoplakia. Early mortality is often associated with bone marrow failure, infections, fatal pulmonary complications, or malignancy. Short-term treatment options for bone marrow failure in patients include anabolic steroids (e.g., oxymetholone), granulocyte macrophage colony-stimulating factor, granulocyte colony-stimulating factor, and erythropoietin. Other treatments include hematopoietic stem cell transplantation (SCT).

Idiopathic pulmonary fibrosis is a chronic and ultimately fatal disease characterized by a progressive decline in lung function. In some appropriate cases, the following agents are used to treat idiopathic pulmonary fibrosis: nintedanib, a tyrosine kinase inhibitor that targets multiple tyrosine kinases, including vascular endothelial growth factor, fibroblast growth factor, and PDGF receptors; and pirfenidone. Other treatments include lung transplantation. In some cases, lung transplantation for idiopathic pulmonary fibrosis (IPF) has been shown to confer a survival benefit over medical therapy.

Generally, a method of treating a telomere disease includes administering a therapeutically effective amount of a compound described herein, to a subject who is in need of, or who has been determined to be in need of, such treatment.

Cancer

The present disclosure also provides compounds, compositions, and methods for treating pre-leukemic conditions, pre-cancerous conditions, dysplasia and/or cancers. Pre-leukemic conditions include, e.g., Myelodysplastic syndrome, and smoldering leukemia. Dysplasia refers to an abnormality of development or an epithelial anomaly of growth and differentiation, including e.g., hip dysplasia, fibrous dysplasia, and renal dysplasia, Myelodysplastic syndromes, and dysplasia of blood-forming cells.

A precancerous condition or premalignant condition is a state of disordered morphology of cells that is associated with an increased risk of cancer. If left untreated, these conditions may lead to cancer. Such conditions are can be dysplasia or benign neoplasia.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells.

Many cancer cells have abnormal telomeres. Thus, treatments described herein (e.g., PAPD5 inhibitors) can also be used to treat cancers. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods described herein are used for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Cancers treatable using the methods described herein are cancers that have increased levels of TERC, an increased expression of genes such as TERC and/or TERT, or increased activity of a telomerase relative to normal tissues or to other cancers of the same tissues.

In some embodiments, the tumor cells isolated from subjects diagnosed with cancer can be used to screen test for compounds that alter TERC levels. In some embodiments, the tumor cells can be used to screen test compounds that alter the expressive or activity of PARN or PAPD5. The cancer cells used in the methods can be, e.g., cancer stem cells. Such methods can be used to screen a library of test compounds, e.g., compounds that alter or change expression of protein or RNA of telomere-associated genes (e.g., TERC, PARN, PAPD5/PAPD5).

In some embodiments, agents that decrease the level or activity of TERC (e.g., PANR inhibitors) are used to treat cancer. In some embodiments, these agents are used in combination with other cancer treatments, e.g., chemotherapies, surgery, or radiotherapy.

Aging

Telomeres shorten over the human life span. In large population based studies, short or shortening telomeres are associated with numerous diseases. Thus, telomeres have an important role in the aging process, and can contribute to various diseases. The role of telomeres as a contributory and interactive factor in aging, disease risks, and protection is described, e.g., in Blackburn, Elizabeth H., Elissa S. Epel, and Jue Lin. “Human telomere biology: A contributory and interactive factor in aging, disease risks, and protection,” Science 350.6265 (2015): 1193-1198, which is incorporated by reference in its entirety.

Telomere attrition is also a major driver of the senescence associated response. In proliferating human cells, progressive telomere erosion ultimately exposes an uncapped free double-stranded chromosome end, triggering a permanent DNA damage response (DDR). The permanent DNA damage response has a profound impact on cell functions. For example, the damage sensor ataxia telangiectasia mutated (ATM) is recruited to uncapped telomeres, leading to the stabilization of tumor suppressor protein 53 (p53) and upregulation of the p53 transcriptional target p21. In turn, p21 prevents cyclin-dependent kinase 2 (CDK2)-mediated inactivation of RB, subsequently preventing entry into the S phase of the cell cycle. Cellular senescence contributes to various age-related diseases, e.g., glaucoma, cataracts, diabetic pancreas, type 2 diabetes mellitus, atherosclerosis, osteoarthritis, inflammation, atherosclerosis, diabetic fat, cancer, pulmonary fibrosis, and liver fibrosis, etc. The permanent DNA damage response and age-related diseases are described, e.g., in Childs, Bennett G., et al. “Cellular senescence in aging and age-related disease: from mechanisms to therapy.” Nature medicine 21.12 (2015): 1424, which is incorporated herein by reference in its entirety.

As used herein, the term “aging” refers to degeneration of organs and tissues over time, in part due to inadequate replicative capacity in stem cells that regenerate tissues over time. Aging may be due to natural disease processes that occur over time, or those that are driven by cell intrinsic or extrinsic pressures that accelerate cellular replication and repair. Such pressures include natural chemical, mechanical, and radiation exposure; biological agents such as bacteria, viruses, fungus, and toxins; autoimmunity, medications, chemotherapy, therapeutic radiation, cellular therapy. As the telomere is an important factor in aging and disease development, the methods described herein can be used for treating, mitigating, or minimizing the risk of, a disorder associated with aging (and/or one or more symptoms of a disorder associated with aging) in a subject. The methods include the step of identifying a subject as having, or being at risk of a disorder associated with aging; and administering a pharmaceutical composition to the subject. In some embodiments, the pharmaceutical composition includes an agent that alters the level or activity of TERC, e.g., increase the level or activity of TERC.

As used herein, the term “disorders associated with aging” or “age-related diseases” refers to disorders that are associated with the ageing process. Exemplary disorders include, e.g., macular degeneration, diabetes mellitus (e.g., type 2 diabetes), osteoarthritis, rheumatoid arthritis, sarcopenia, cardiovascular diseases such as hypertension, atherosclerosis, coronary artery disease, ischemia/reperfusion injury, cancer, premature death, as well as age-related decline in cognitive function, cardiopulmonary function, muscle strength, vision, and hearing.

The disorder associated with aging can also be a degenerative disorder, e.g., a neurodegenerative disorder. Degenerative disorders that can be treated or diagnosed using the methods described herein include those of various organ systems, such as those affecting brain, heart, lung, liver, muscles, bones, blood, gastrointestinal and genito-urinary tracts. In some embodiments, degenerative disorders are those that have shortened telomeres, decreased levels of TERC, and/or decreased levels of telomerase relative to normal tissues. In some embodiments, the degenerative disorder is a neurodegenerative disorder. Exemplary neurodegenerative disorders include Motor Neuron Disease, Creutzfeldt-Jakob disease, Machado-Joseph disease, Spino-cerebellar ataxia, Multiple sclerosis (MS), Parkinson's disease, Alzheimer's disease, Huntington's disease, hearing and balance impairments, ataxias, epilepsy, mood disorders such as schizophrenia, bipolar disorder, and depression, dementia, Pick's Disease, stroke, CNS hypoxia, cerebral senility, and neural injury such as head trauma. Recent studies have shown the association between shorter telomeres and Alzheimer's disease. The relationship between telomere length shortening and Alzheimer's disease is described., e.g., in Zhan, Yiqiang, et al. “Telomere length shortening and Alzheimer disease—a Mendelian Randomization Study,” JAMA neurology 72.10 (2015): 1202-1203, which is incorporated by reference in its entirety. In some embodiments, the neurodegenerative disorder is dementia, e.g., Alzheimer's disease.

It has also been determined that there an inverse association between leucocyte telomere length and risk of coronary heart disease. This relationship is described, e.g., in Haycock, Philip C., et al. “Leucocyte telomere length and risk of cardiovascular disease: systematic review and meta-analysis.” (2014): g4227; and Codd, Veryan, et al. “Identification of seven loci affecting mean telomere length and their association with disease.” Nature genetics 45.4 (2013): 422-427; each of which is incorporated by reference in its entirety. Thus, there is strong evidence for a causal role of telomere-length variation in cardiovascular disease (CVD), or coronary artery disease (CAD). In some embodiments, the disorder is a cardiovascular disease (CVD), and/or coronary artery disease (CAD), and the present disclosure provides methods of treating, mitigating, or minimizing the risk of, these disorders. In some cases, the disorder is an atherosclerotic cardiovascular disease.

Furthermore, a meta-analysis of 5759 cases and 6518 controls indicated that shortened telomere length was significantly associated with type 2 diabetes mellitus risk. The relationship between telomere length and type 2 diabetes mellitus is described, e.g., in Zhao, Jinzhao, et al. “Association between telomere length and type 2 diabetes mellitus: a meta-analysis.” PLoS One 8.11 (2013): e79993, which is incorporated by reference in its entirety. In some embodiments, the disorder is a metabolic disorder, e.g., type 2 diabetes mellitus.

In some embodiments, aged cells can be used to screen test compounds that alter the expressive or activity of PARN or PAPD5. The aged cells used in the methods can be, e.g., those with genetic lesions in telomere biology genes, those isolated from elderly subjects, or those that undergo numerous rounds of replication in the lab. Such methods can be used to screen a library of test compounds, e.g., compounds that alter or change expression of protein or RNA of telomere-associated genes (e.g., TERC, PARN, PAPD5/PAPD5). Exemplary methods of screening and screening techniques are described herein.

In some embodiments, agents that increase the level or activity of TERC (e.g., PAPD5/PAPD5 inhibitors) are used to treat age-related degenerative disorders due to natural causes or environmental causes. In some embodiments, these agents are used in combination with other treatments.

Diagnosing a Subject in Need of Treatment

The present specification provides methods of diagnosing a subject in need of treatment (e.g., as having any one of telomere diseases described herein). As an example, if the level or activity of TERC, PARN, and/or PAPD5 in a subject is comparable to the level or activity of TERC, PARN, and/or PAPD5 in a subject having the telomere disease and, optionally, the subject has one or more symptoms associated with telomere disease (e.g., aplastic anemia, pulmonary fibrosis, hepatic cirrhosis), then the subject can be diagnosed as having or being at risk of developing a telomere disease.

In some embodiments, if the level or activity of TERC, PARN, and/or PAPD5 in a subject is comparable to the level or activity of TERC, PARN, and/or PAPD5 in a control subject who does not have a telomere disease, then the subject can be diagnosed as not having telomere disease or not being at risk of developing a telomere disease.

In some embodiments, the subject is determined to have or being at risk of developing a telomere disease if there is a mutation at PARN. The mutation can be a deletion containing part of PARN gene or the entire PARN gene. The mutation can also be a mutation at position 7 and/or 87 of PARN, e.g., the amino acid residue at position 7 is not asparagine, and/or the amino acid residue at position 87 of PARN is not serine. For example, the mutation can be a missense variant c.19A>C, resulting in a substitution of a highly conserved amino acid p.Asn7His. In some cases, the mutation is a missense mutation c.260C>T, encoding the substitution of a highly conserved amino acid, p. Ser87Leu.

In some embodiments, a subject has no overt signs or symptoms of a telomere disease, but the level or activity of TERC, PARN or PAPD5 may be associated with the presence of a telomeres disease, then the subject has an increased risk of developing telomere disease. In some embodiments, once it has been determined that a person has telomere disease, or has an increased risk of developing telomere disease, then a treatment, e.g., with a small molecule (e.g., a PAPD5 inhibitor) or a nucleic acid encoded by a construct, as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of PAPD5 protein, e.g., a control reference level that represents a normal level of PAPD5 protein, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia). In another embodiment, the reference comprises a predetermined value for a meaningful level of PARN protein, e.g., a control reference level that represents a normal level of PARN protein, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein. In some embodiments, it may be desirable that the control subject is deficient in PARN gene (e.g., Dyskeratosis Congenita), and in other embodiments, it may be desirable that a control subject has cancer. In some cases, it may be desirable that the control subject has high telomerase activity, and in other cases it may be desirable that a control subject does not have substantial telomerase activity.

In some embodiments, the level of TERC or PARN in a subject being less than or equal to a reference level of TERC or PARN is indicative of a clinical status (e.g., indicative of a disorder as described herein, e.g., telomere disease). In some embodiments, the activity of TERC or PARN in a subject being greater than or equal to the reference activity level of TERC or PARN is indicative of the absence of disease.

The predetermined value can depend upon the particular population of subjects (e.g., human subjects or animal models) selected. For example, an apparently healthy population will have a different ‘normal’ range of levels of TERC than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art. In characterizing likelihood, or risk, numerous predetermined values can be established.

In some embodiments, the methods described in this disclosure involves identifying a subject as having, being at risk of developing, or suspected of having a disorder associated with telomerase dysfunction. The methods include determining the level or activity of TERC, PARN, or PAPD5 in a cell from the subject; comparing the level or activity of TERC, PARN, or PAPD5 to the reference level or reference activity of TERC, PARN, or PAPD5; and identifying the subject as having, being at risk of developing, or suspected of having a disorder associated with telomerase dysfunction if the level or activity of TERC, PARN, or PAPD5 is significantly different from the reference level or activity of TERC, PARN, or PAPD5. In some embodiments, the reference level or activity of TERC, PARN, or PAPD5 are determined by cells obtained from subjects without disorders associated with telomerase dysfunction.

The level or activity of TERC, PARN, or PAPD5 can be determined in various types of cells from a subject. The methods can include obtaining cells from a subject, and transforming these cells to induced pluripotent stem cells (iPS) cells, and these iPS cells can be used to determine the level or activity of TERC, PARN, or PAPD5. These cells can be, e.g., primary human cells or tumor cells. Pluripotent stem cells (iPS) cells can be generated from somatic cells by methods known in the art (e.g., somatic cells may be genetically reprogrammed to an embryonic stem celllike state by being forced to express genes and factors important for maintaining the defining properties of embryonic stem cells). In some embodiments, the methods of diagnosing a subject include analyzing blood sample of the subject, or a sample of hair, urine, saliva, or feces of the subject (e.g., a subject may be diagnosed without any cell culture surgically obtained from the subject).

The subject may be one having a mutation at PARN, e.g., a deletion containing part of PARN gene or the entire PARN gene. For example, the mutation may be one wherein the amino acid residue at position 7 of PARN is not asparagine or serine. For example, the subject can have a missense variant c.19A>C, resulting in a substitution of a highly conserved amino acid p.Asn7His. The subject can have a missense mutation c.260C>T, encoding the substitution of a highly conserved amino acid, p. Ser87Leu.

Induced Pluripotent Stem Cells

Induced pluripotent stem cells (iPSC or iPS), are somatic cells (e.g., derived from patient skin or other cell) that have been genetically reprogrammed to an embryonic stem cell-like state by being forced to express genes and factors important for maintaining the defining properties of embryonic stem cells. These cells can be generated by methods known in the art.

It is known that mouse iPSCs demonstrate important characteristics of pluripotent stem cells, including expressing stem cell markers, forming tumors containing cells from all three germ layers, and being able to contribute to many different tissues, when injected into mouse embryos at a very early stage in development.

Human iPSCs also express stem cell markers and are capable of generating cells characteristic of all three germ layers. iPSCs can be generated from human fibroblasts and are already useful tools for drug development and modeling of diseases. Viruses are currently used to introduce the reprogramming factors into adult cells (e.g., lentiviral vectors disclosed herein), and this process can be carefully controlled and tested in cultured, isolated cells first to then treat cells (e.g., by contacting with a test compound) to express altered markers, e.g., iPSCs from tumor cells can be manipulated to differentiate or iPSCs from cardiomyocytes can be manipulated to de-differentiate.

The iPSC manipulation strategy can be applied to any cells obtained from a subject to test whether the compound can alter the level or activity of TERC, PARN, or PAPD5. The cells are contacted with test compounds (e.g., small molecules). In some embodiments, these iPSC cells can be used for screening compounds that modulate TERC. In some embodiments, the iPSC cells can be converted from patient skin fibroblasts.

Cell Expansion

The present disclosure also provides methods of expanding a cell population by culturing one or more cells in the presence of compounds as disclosed herein. In some embodiments, cell expansion can involve contacting the cells with an effective amount of compound of the present disclosure (e.g., PAPD5 inhibitor compounds of Formula (I) or Formula (II)). The PAPD5 inhibitors can decrease the level and activity of PAPD5, thereby increasing or maintaining the length of the telomere. Telomerase activity and telomere length maintenance are related to cell expansion capability. As the cell divides, the telomere length gradually shortens, eventually leading to cell senescence of cells. Based on the telomere theory, aging in cells is irreversible. Programmed cell cycle arrest happens in response to the telomerase activity and the total number of cell divisions cannot exceed a particular limit termed the Hayflick limit. It has been determined that maintaining telomere length during cell replication is important for cell expansion (e.g., stem cell expansion). The present disclosure provides methods of promoting cell expansion, and methods of inhibiting, slowing, or preventing cell aging.

In some embodiments, the cell is a stem cell. Stem cells can include, but are not limited to, for example, pluripotent stem cells, embryonic stem cells, hematopoietic stem cells, adipose derived stem cells, mesenchymal stem cells, umbilical cord blood stem cells, placentally derived stem cells, exfoliated tooth derived stem cells, hair follicle stem cells, or neural stem cells. In some embodiments, the cell is a peripheral blood mononuclear (PBMC) cell such as a lymphocyte, that may be further engineered into a cellular therapy.

The cells can be derived from the subject with a disease or condition associated with any disorder described herein, e.g., cancer, a telomere or telomerase dysfunction, a disorder associated with aging, a pre-leukemic or pre-cancerous condition, and a neurodevelopment disorder. The cells can be isolated and derived, for example, from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

The cells can be isolated from any mammalian organism, e.g., human, mouse, rats, dogs, or cats, by any means know to one of ordinary skill in the art. One skilled in the art can isolate embryonic or adult tissues and obtain various cells (e.g., stem cells).

The expanded cell population can be further enriched by using appropriate cell markers. For example, stem cells can be enriched by using specific stem cell markers, e.g., FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, SSEA-4, and Sox-2. One skilled in the art can enrich a specific cell population by using antibodies known in the art against any of these cell markers. In some embodiments, expanded stem cells can be purified based on desired stem cell markers by fluorescence activated cell sorting (FACS), or magnet activated cell sorting (MACS).

The cells (e.g., stem cells) can be cultured and expanded in suitable growth media. Commonly used growth media include, but are not limited to, Iscove's modified Dulbecco's Media (IMDM) medium, McCoy's 5A medium, Dulbecco's Modified Eagle medium (DMEM), KnockOut™ Dulbecco's Modified Eagle medium (KO-DMEM), Dulbecco's Modified Eagle Medium/Nutrient Mixture F-12 (DMEM/F12), Roswell Park Memorial Institute (RPMI) medium, minimum essential medium alpha medium (α-MEM), F-12K nutrient mixture medium (Kaighn's modification, F-12K), X-vivo™ 20 medium, Stemline™ medium, StemSpan™ CC100 medium, StemSpan™ H2000 medium, MCDB 131 Medium, Basal Media Eagle (BME), Glasgow Minimum Essential medium (GMEM), Modified Eagle Medium (MEM), Opti-MEM I Reduced Serum medium, Waymouth's MB 752/1 Medium, Williams' Medium E, NCTC-109 Medium, neuroplasma medium, BGJb Medium, Brinster's BMOC-3 Medium, Connaught Medical Research Laboratories (CMRL) Medium, CO₂-Independent Medium, and Leibovitz's L-15 medium.

The compounds of the present disclosure can be used to expand various cell population, e.g., by adding the compound in cell culture media in a tube or plate. The concentration of the compound can be determined by, but limited to, the time of cell expansion. For example, the cells can be in culture with high concentration of the compound for a short period of time, e.g., at least or about 1 day, 2 days, 3 days, 4 days, or 5 days. In some embodiments, the cells can be cultured with a low concentration of the compound for a long period of time, e.g., at least or about 3 days, 4 days, 5 days, 6 days, 1 week, 2 weeks, 3 weeks, or 4 weeks.

In some embodiments, growth factors are also added to the growth medium to expand cells. Examples of suitable growth factors include, but are not limited to, thrombopoietin, stem cell factor, IL-1, IL-3, IL-7, flt-3 ligand, G-CSF, GM-CSF, Epo, FGF-1, FGF-2, FGF-4, FGF-20, IGF, EGF, NGF, LIF, PDGF, bone morphogenic proteins, activin-A, VEGF, forskolin, and glucocorticords. Further, one skilled in the art, using methods known in the art, can add a feeder layer to the culture medium. A feeder layer can include cells such as, placental tissue or cells thereof.

The methods described herein can also be used to produce and expand Chimeric Antigen Receptor (CAR) T-Cells. CAR-T cell therapies involve genetic modification of patient's autologous T-cells to express a CAR specific for a tumor antigen, following by ex vivo cell expansion and re-infusion back to the patient. PBMCs can be collected from a patient and cultured in the presence of the compounds as described herein, with appropriate media (e.g., complete media containing 30 U/mL interleukin-2 and anti-CD3/CD28 beads). The cells can be expanded for about 3 to 14 days (e.g., about 3 to 7 days). Subsets of T cells can be sorted by FACS. Gating strategies for cell sorting can exclude other blood cells, including granulocytes, monocytes, natural killer cells, dendritic cells, and B cells. Primary T cells are then transduced by incubating cells with the CAR-expressing lentiviral vector in the culture media. In some embodiments, the culture media can be supplemented with the compounds as described herein. The transduced cells are then cultured for at least a few days (e.g., 3 days) before being used in CAR-T cell therapies. In some embodiments of these methods, the cell is a Chimeric Antigen Receptor (CAR) T-Cell. In some embodiments, the cell is a lymphocyte. In some embodiments, the cell is a T cell, an engineered T cell, or a natural killer cell (NK). In some embodiments, the cell is a T cell. In some embodiments, the cell is an engineered T cell. In some embodiments, the cell is a natural killer cell (NK).

Additional Uses

In some embodiments, the compound of the present disclosure modulates RNAs whose transcription, post-transcriptional processing, stability, steady state levels or function are altered due to acquired or genetic defects in one or more of any cellular pathways. In some embodiments, these include non-coding RNAs (ncRNAs) that are members of the small nucleolar RNA (snoRNA), small Cajal body RNA (scaRNA), small nuclear RNA (snRNA), ribosomal RNA (rRNA), Y RNA, transfer RNA (tRNA), microRNA (miRNA), PIWI-interacting RNA (piRNA) or long non-coding RNA (lncRNA) families. The compounds may also by useful for modulating non-coding RNAs in a cell (e.g. scaRNA13, scaRNA8), and concomitantly for preventing and treating the associated disease and conditions. In some embodiments, these also include those ncRNAs affected by any of the molecular mechanisms described, for example, in Lardelli et al, Nature Genetics, 49(3), 2017, 457-464; and in Son et al., 2018, Cell Reports 23, 888-898, including those affected by disruption of PARN or TOE1 deadenylases. As such, the compounds are useful in treating or preventing genetic and other disorders, including neurodevelopmental disorders such as pontocerebellar hypoplasia. Neurodevelopmental disorders are a group of disorders in which the development of the central nervous system is disturbed. This can include developmental brain dysfunction, which can manifest as neuropsychiatric problems or impaired motor function, learning, language or non-verbal communication. In some embodiments, a neurodevelopmental disorder is selected from attention deficit hyperactivity disorder (ADHD), reading disorder (dyslexia), writing disorder (disgraphia), calculation disorder (dyscalculia), expression disorder (ability for oral expression is substantially below the appropriate level for a child's mental age), comprehension disorder (ability for comprehension is markedly below the appropriate level for a child's mental age), mixed receptive-expressive language disorder, speech disorder (dislalia) (inability to use the sounds of speech that are developmentally appropriate), stuttering (disruption of normal fluency and temporal structure of speech), and autism spectrum disorders (persistent difficulties in social communication). In some embodiments, the present disclosure provides a method of treating an acquired or genetic disease or condition associated with alterations in RNA, the method comprising administering to the subject in need thereof a therapeutically effective amount of any one of the compounds described herein, or a pharmaceutically acceptable salt thereof, or a pharmaceutically acceptable composition comprising same. In some embodiments, the RNA comprises ncRNA (e.g., snRNA, scaRNA, snoRNA, rRNA, and miRNA). In some embodiments, the RNA is disrupted by disruption of PARN or TOE1 deadenylase. In some embodiments, the acquired or genetic disease or condition associated with alterations in RNA comprises a neurodevelopmental disorder such as pontocerebellar hypoplasia.

Because the compounds are PAPD5 inhibitors, and because these affect TERC, telomerase, telomere maintenance and stem cell self-renewal, the compounds are useful in modulating ex vivo expansion of stem cells, and also useful for allograft exhaustion, in hematopoietic or other tissues. For example, PAPD5 inhibitors may be useful for the ex vivo expansion of hematopoietic stem cells as described in Fares, et al, 2015, Science 345, 1590-1512, and Boitano, et al, 2010 329, 1345-1348, both of which are incorporated by reference herein in their entireties.

Pharmaceutical Compositions and Formulations

The present application also provides pharmaceutical compositions comprising an effective amount of a compound of any one of the Formulae disclosed herein, or a pharmaceutically acceptable salt thereof, and a pharmaceutically acceptable carrier. The pharmaceutical composition can also comprise at least one of any one of the additional therapeutic agents described herein. In certain embodiments, the application also provides pharmaceutical compositions and dosage forms comprising any one of the additional therapeutic agents described herein (e.g., in a kit). The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that can be used in the pharmaceutical compositions of the present application include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain any one of the compounds and therapeutic agents described herein in the range of 0.005% to 100% with the balance made up from the suitable pharmaceutically acceptable excipients. The contemplated compositions can contain 0.001%-100% of any one of the compounds and therapeutic agents provided herein, in one embodiment 0.1-95%, in another embodiment 75-85%, in a further embodiment 20-80%, wherein the balance can be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein can conveniently be presented in a unit dosage form, e.g., tablets, capsules (e.g., hard or soft gelatin capsules), sustained release capsules, and in liposomes, and can be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration can be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which can beneficially increase the rate of compound absorption. In the case of tablets for oral use, carriers that are commonly used include lactose, sucrose, glucose, mannitol, and silicic acid and starches. Other acceptable excipients can include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents can be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which can contain antioxidants, buffers, bacteriostats and solutes which render the formulation isotonic with the blood of the intended recipient; and aqueous and non-aqueous sterile suspensions which can include suspending agents and thickening agents. The formulations can be presented in unit-dose or multi-dose containers, for example, sealed ampules and vials, and can be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions can be prepared from sterile powders, granules and tablets. The injection solutions can be in the form, for example, of a sterile injectable aqueous or oleaginous suspension. This suspension can be formulated according to techniques known in the art using suitable dispersing or wetting agents and suspending agents. The sterile injectable preparation can also be a sterile injectable solution or suspension in a non-toxic parenterally-acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant.

The pharmaceutical compositions of the present application can be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include cocoa butter, beeswax, and polyethylene glycols.

The pharmaceutical compositions of the present application can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application can be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polydimethylsiloxane, polycaprolactone, polyethylene glycol, polylactic acid, ethylene vinyl acetate, and mixtures thereof. The coatings can optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a therapeutic compound is present in an effective amount (e.g., a therapeutically effective amount).

Effective doses can vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a therapeutic compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0. 1 mg/kg to about 200 mg/kg; from about 0. 1 mg/kg to about 150 mg/kg; from about 0. 1 mg/kg to about 100 mg/kg; from about 0.1 mg/kg to about 50 mg/kg; from about 0. 1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a therapeutic compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). The compounds and compositions described herein can be administered to the subject in any order. A first therapeutic agent, such as a compound of Formula (I), can be administered prior to or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before or after), or concomitantly with the administration of a second therapeutic agent, such as an anti-cancer therapy described herein, to a subject in need of treatment. Thus, the compound of Formula (I), or a composition containing the compound, can be administered separately, sequentially or simultaneously with the second therapeutic agent, such as a chemotherapeutic agent described herein. When the compound of Formula (I), or a pharmaceutically acceptable salt thereof, and a second or third therapeutic agent are administered to the subject simultaneously, the therapeutic agents can be administered in a single dosage form (e.g., tablet, capsule, or a solution for injection or infusion).

Combination Therapies

In some embodiments, the compounds described here may be administered to a subject in any combination with treatments for telomere diseases that are known in the art. The combination treatment may be administered to the subject either consecutively or concomitantly with the compound of any one of the Formula disclosed herein. When combination treatment comprises an alternative therapeutic agent, the therapeutic agent may be administered to the subject in any one of the pharmaceutical compositions described herein.

In some embodiments, the compounds of the present disclosure may be used in combination with a therapeutic agent that is useful in treating a telomere disease (e.g., a therapeutic agent that modulates the level or activity of TERC). In some embodiments, the agent useful in treating a telomere disease is a nucleic acid comprising a nucleotide sequence that encodes PARN. The agent can also be an anti-PARN antibody or anti-PARN antibody fragment. In some embodiments, the agent is an antisense molecule or a small interfering nucleic acid which is specific for a nucleic acid encoding PARN. In some embodiments, the agent is a nucleic acid comprising a nucleotide sequence that encodes PAPD5. The agent can also be an anti-PAPD5 antibody or anti-PAPD5 antibody fragment. In some embodiments, the agent is an antisense molecule or a small interfering nucleic acid which is specific for a nucleic acid encoding PAPD5. The antisense molecule described herein can be an oligonucleotide. In some cases, the agent binds to PARN or PAPD5.

In some embodiments, the therapeutic agent that is useful in treating a telomere disease is selected from adenosine analogues, aminoglycosides, and purine nucleotides, etc. In some cases, the aminoglycoside can be a member of the neomycin and kanamycin families. The aminoglycoside can be, for example, kanamycin B sulfate, pramycin sulfate, spectinomycin dihydrochloride pentahydrate, ribostamycin sulfate, sisomicin sulfate, amikacin disulfide, dihydrostreptomycin sesquisulfate, hygromycin B, netilmicin sulfate, paromomycin sulfate, kasugamycin, neomycin, gentamicin, tobramycin sulfate, streptomycin sulfate, or neomycin B, or derivatives thereof.

In some embodiments, the therapeutic agent that is useful in treating a telomere disease a nucleoside analogue, e.g., an adenosine analogue, 8-chloroadenosine (8-Cl-Ado) and 8-aminoadenosine (8-amino-Ado), or the triphosphate derivative thereof, synthetic nucleoside analogue bearing a fluoroglucopyranosyl sugar moiety, benzoyl-modified cytosine or adenine, adenosine- and cytosine-based glucopyranosyl nucleoside analogue, or glucopyranosyl analogue bearing uracil, 5-fluorouracil or thymine, etc.

Adenosine analogues, aminoglycosides, and purine nucleotides are known in the art, and they are described, e.g., in Kim, Kyumin, et al. “Exosome Cofactors Connect Transcription Termination to RNA Processing by Guiding Terminated Transcripts to the Appropriate Exonuclease within the Nuclear Exosome.” Journal of Biological Chemistry (2016): jbc-M116; Chen, Lisa S., et al. “Chain termination and inhibition of mammalian poly (A) polymerase by modified ATP analogues.” Biochemical pharmacology 79.5 (2010): 669-677; Ren, Yan-Guo, et al. “Inhibition of Klenow DNA polymerase and poly (A)-specific ribonuclease by aminoglycosides.” Rna 8.11 (2002): 1393-1400; Thuresson, Ann-Charlotte, Leif A. Kirsebom, and Anders Virtanen. “Inhibition of poly (A) polymerase by aminoglycosides.” Biochimie 89.10 (2007): 1221-1227; AA Balatsos, N., et al. “Modulation of poly (A)-specific ribonuclease (PARN): current knowledge and perspectives.” Current medicinal chemistry 19.28 (2012): 4838-4849; Balatsos, Nikolaos AA, Dimitrios Anastasakis, and Constantinos Stathopoulos. “Inhibition of human poly (A)-specific ribonuclease (PARN) by purine nucleotides: kinetic analysis.” Journal of enzyme inhibition and medicinal chemistry 24.2 (2009): 516-523; Balatsos, Nikolaos AA, et al. “Competitive inhibition of human poly (A)-specific ribonuclease (PARN) by synthetic fluoro-pyranosyl nucleosides.” Biochemistry 48.26 (2009): 6044-6051; and Balatsos, Nikolaos, et al. “Kinetic and in silico analysis of the slow-binding inhibition of human poly (A)-specific ribonuclease (PARN) by novel nucleoside analogues.” Biochimie 94.1 (2012): 214-221; each of which is incorporated herein by reference in its entirety. Numerous therapeutic agents that can modulate the level or activity of PARN and/or PAPD5 are described, e.g., in WO 2017/066796, which is incorporated herein by reference in its entirety.

In some embodiments, the compounds of the present disclosure are used in combination with an anti-cancer therapy. In some embodiments, the anti-cancer therapy is selected from the group consisting of surgery, radiation therapy, chemotherapy, gene therapy, DNA therapy, viral therapy, RNA therapy, adjuvant therapy, and immunotherapy. In some embodiments, the anti-cancer therapy is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite. In some embodiments, the anti-cancer therapy is an ataxia telangiectasia mutated (ATM) kinase inhibitor. Suitable examples of platinum agents include cisplatin, carboplatin, oxaliplatin, satraplatin, picoplatin, nedaplatin, triplatin, and lipoplatin. Suitable examples of cytotoxic radioisotopes include ⁶⁷Cu, ⁶⁷Ga, ⁹⁰Y, ¹³¹I, ¹⁷⁷Lu, ¹⁸⁶Re, ¹⁸⁸Re, α-Particle emitter, ²¹¹At, ²¹³Bi, ²²⁵Ac, Auger-electron emitter, ¹²⁵I, ²¹²Pb, and ¹¹¹In. Suitable examples of antitumor alkylating agents include nitrogen mustards, cyclophosphamide, mechlorethamine or mustine (HN₂), uramustine or uracil mustard, melphalan, chlorambucil, ifosfamide, bendamustine, nitrosoureas, carmustine, lomustine, streptozocin, alkyl sulfonates, busulfan, thiotepa, procarbazine, altretamine, triazenes, dacarbazine, mitozolomide, and temozolomide. Suitable examples of anti-cancer monoclonal antibodies include to necitumumab, dinutuximab, nivolumab, blinatumomab, pembrolizumab, ramucirumab, obinutuzumab, adotrastuzumab emtansine, pertuzumab, brentuximab, ipilimumab, ofatumumab, catumaxomab, bevacizumab, cetuximab, tositumomab-I¹³¹, ibritumomab tiuxetan, alemtuzumab, gemtuzumab ozogamicin, trastuzumab, and rituximab. Suitable examples of vinca alkaloids include vinblastine, vincristine, vindesine, vinorelbine, desoxyvincaminol, vincaminol, vinburnine, vincamajine, vineridine, vinburnine, and vinpocetine. Suitable examples of antimetabolites include fluorouracil, cladribine, capecitabine, mercaptopurine, pemetrexed, fludarabine, gemcitabine, hydroxyurea, methotrexate, nelarbine, clofarabine, cytarabine, decitabine, pralatrexate, floxuridine, and thioguanine.

Kits

The present disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit can optionally include directions to perform a test to determine that a subject is in need of treatment with a compound of any one of the Formulae as described herein, and/or any of the reagents and device(s) to perform such tests. The kit can also optionally include an additional therapeutic agent (e.g., a nucleic acid comprising a nucleotide sequence that encodes PARN or PAPD5).

### EXAMPLES

### Example 1

### Inhibition of Recombinant PAPD5

Recombinant PAPD5 as well as catalytically inactive mutant PAPD5 (D189A, D191A) were purified for in vitro assays. An in vitro RNA polyadenylation assay using recombinant PAPD5, ATP and an oligonucleotide substrate utilized the following phenomenon: ATP utilization by PAPD5 reads out as a decreased luminescence signal produced by luciferase (KinaseGlo, Promega, Madison, Wis.).

0.25 μl of PAPD5 in a buffer composition at a concentration of 50 nM was added to a well of a microtitre plate (e.g., Product #3820; non-binding surface; Corning Incorporated, Corning, N.Y.) using a Thermo MultiDrop Combi (Thermo Fisher Scientific, Waltham, Mass.). For positive control (e.g., wells A24:P24 in a multi-well plate), 0.5 μl of mutant PAPD5 was added at a concentration of 50 nM.

100 nl of a compound dissolved in DMSO was transferred to each well of the assay plate via pin transfer. For negative control wells, DMSO alone was added. Plates were gently vortexed for 5 seconds, then incubated for 2 hours at room temperature.

After 2 hours, 5 μl of luciferase (Promega KinaseGlo, Madison, Wis.) was added using a MultiDrop Combi (Thermo Fisher Scientific, Waltham, Mass.). The mixture was gently vortexed for 5 seconds and incubated for 10 minutes at room temperature. Plates were spun for 1 minute prior to luminescence measurements. Luminescent measurements were quantitated using a PerkinElmer EnVision™ plate reader.

The fold-change for 100 μM compound and 33 μM compound were calculated. For certain compounds, fold-change at 10 μM, 3.3 μM, and 1 μM concentration was also determined. The fold change is a ratio of luminescence from a sample with inhibitor compared to that with DMSO (a higher number indicates higher inhibition).

### Example 2

### Bioactivity of DHQ-1

As used herein, DHQ-1 (also known as DHQ or RG7834) refers to (S)-6-isopropyl-10-methoxy-9-(3-methoxypropoxy)-2-oxo-6,7-dihydro-2H-pyrido[2,1-a]isoquinoline-3-carboxylic acid having formula:

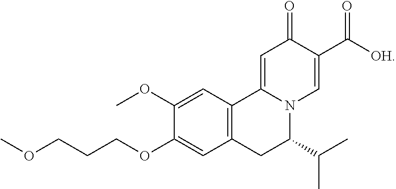 [SMILES: COCCCOc1cc2c(cc1OC)-c1cc(=O)c(C(=O)O)cn1[C@H](C(C)C)C2]

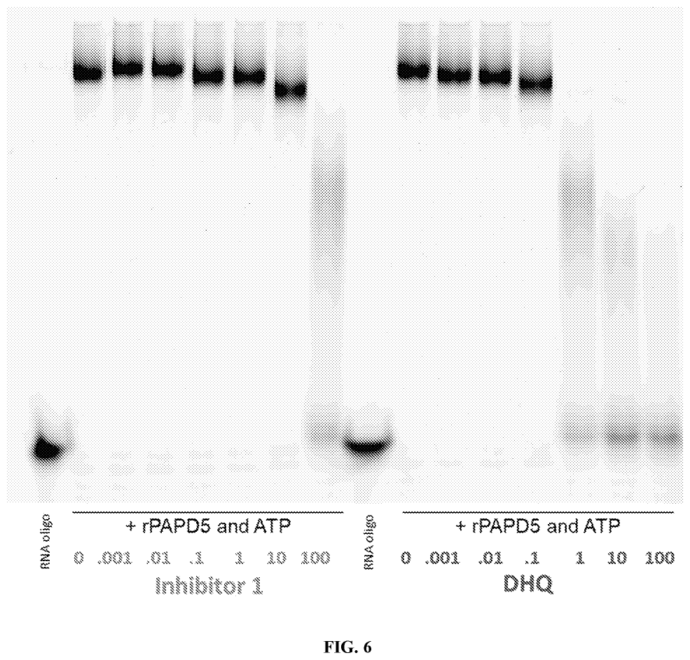

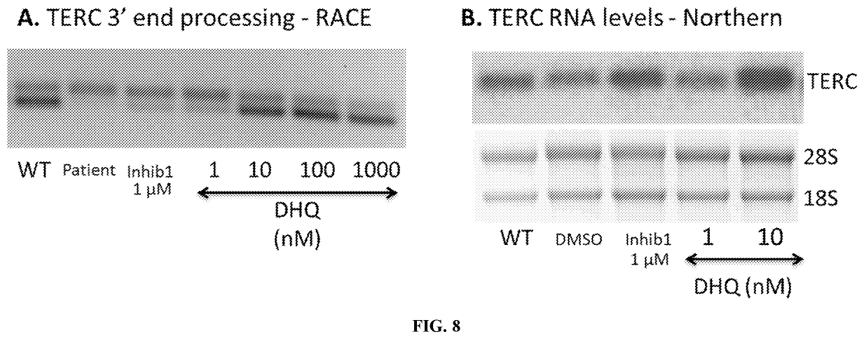

DHQ1 may be used in the prevention and treatment of telomere diseases and other indications described herein (see FIG. 6: in vitro RNA oligo extension; FIG. 8: TERC RACE and TERC levels).

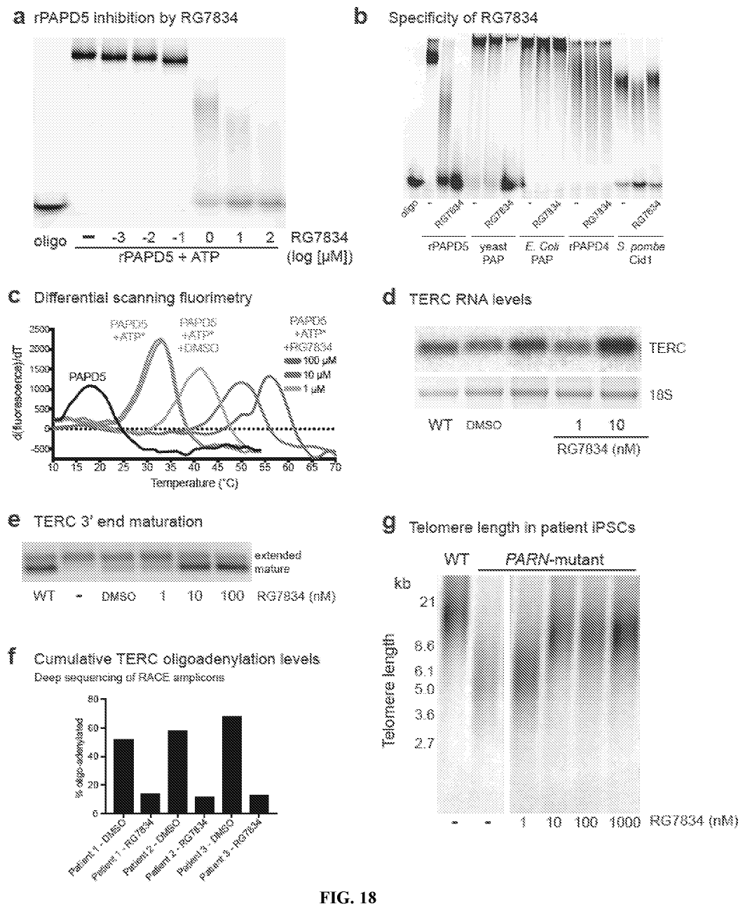

A small molecule HBSAg inhibitor DHQ-1 emerging from functional screening is shown to bind PAPD5 and the related polymerase PAPD7 in yeast three hybrid experiments, but not directly demonstrated(1). The data presented herein shows that DHQ-1 inhibit rPAPD5 in vitro. DHQ-1 binds directly to rPAPD5, inhibits RNA polyadenylation in vitro, and has selectivity for rPAPD5 compared to other polymerases, an exception being yeast poly(A) polymerase (FIG. 18a-c). Interestingly, when PARN-mutant DC patient iPSCs was treated with DHQ-1, the restored TERC 3′-end maturation, TERC levels, and telomere length were observed (FIG. 18d-g). These data provide further evidence that small molecules targeting PAPD5 restore TERC and telomere length in human cells.

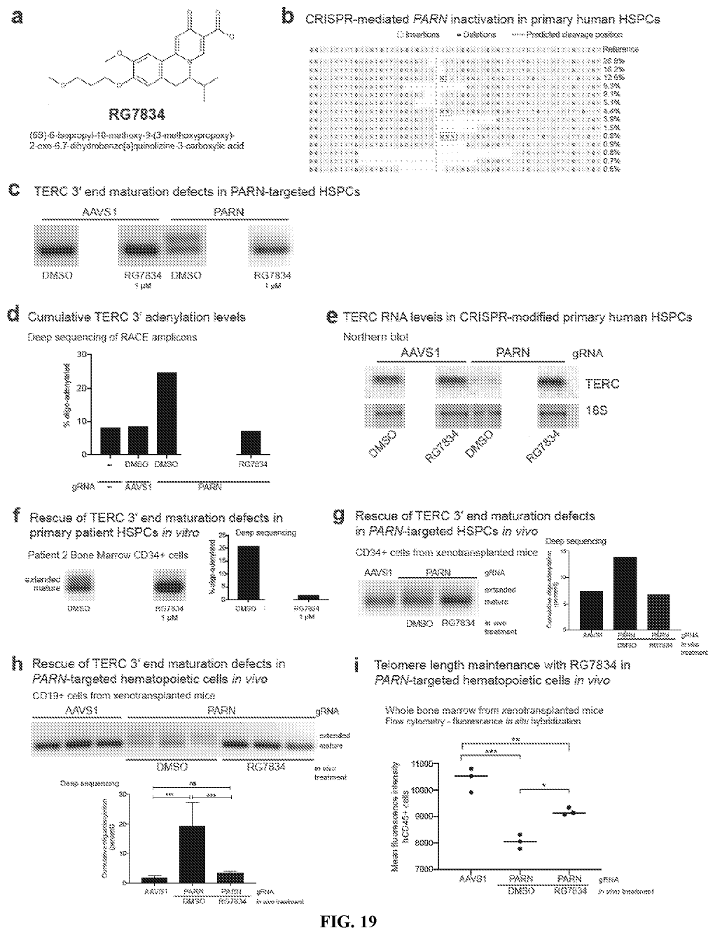

Next, it was shown that PAPD5 inhibition could impact telomere biology in disease-relevant human cells. Bone marrow failure occurs at high frequency in DC. This is driven largely by a cell-intrinsic telomere maintenance defect in hematopoietic stem cells (HSCs), as evidenced by the curative effects of allogeneic bone marrow transplantation, and the selection of somatically reverted TERC mutations in the blood in vivo. The data presented here shows that it is possible to restore TERC defects in primary human CD34⁺ cells, which are enriched for HSCs, by treatment with PAPD5 inhibitors. Using an optimized CRISPR/Cas9 ribonucleoprotein gene-editing strategy(2), high (≥85%) indel frequencies of target genes were obtained in mobilized peripheral blood CD34⁺ cells from healthy volunteers (FIG. 19b). After 5 days of in vitro culture, extended TERC forms were found specifically in PARN-targeted CD34⁺ cells, which were absent in cells targeted with both PARN and PAPD5 gRNAs (FIG. 19c). When PARN-deficient CD34⁺ cells were treated with DHQ-1, decreased TERC 3′ adenylation and increased TERC RNA levels were observed (FIG. 19c-e).

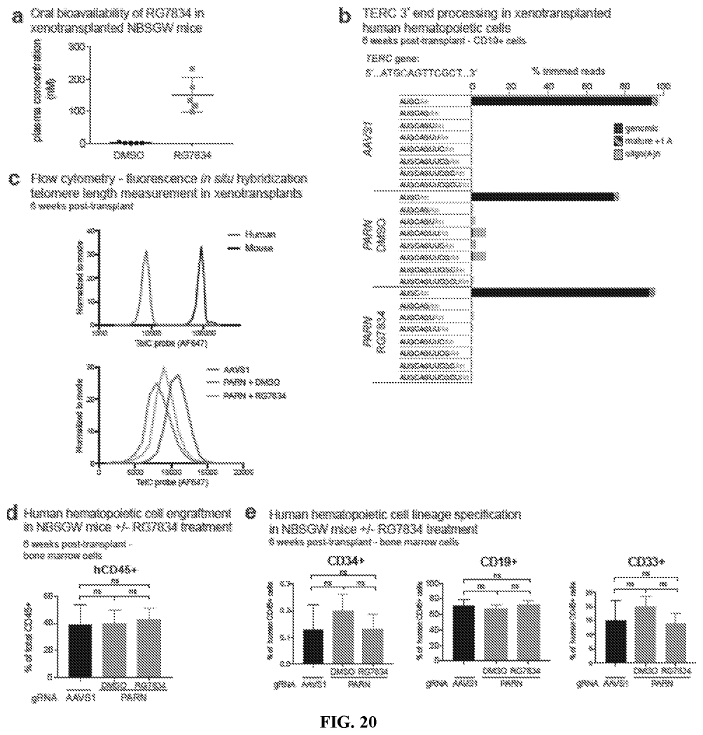

Primary PARN-mutant patient-derived CD34⁺ cells and bone marrow mononuclear cells (BMMC) were studied next. CD34⁺ cell numbers were limited due to the patients' bone marrow failure. To overcome this in vitro hematopoietic differentiation of patient-derived CD34⁺ cells in methylcellulose was performed in the presence or absence of DHQ-1 or vehicle, and found restoration of TERC 3′-end processing specifically in the presence of PAPD5 inhibitors (FIG. 19f). Next it was determined that PAPD5 inhibitors could rescue TERC and telomere length in vivo. Primary human CD34⁺ cells in which PARN was targeted by CRISPR/Cas9 were transplanted into immunodeficient NOD,B6.SCID Il2rγ^{-/-} Kit^{W41/W41} (NBSGW) mice(3). Mice were treated with DHQ-1, which is orally bioavailable(4) (FIG. 20a). Six to eight weeks after xenotransplant, when human CD45+ cells engrafted in the bone marrow were analyzed, evidence of restored TERC maturation in DHQ-treated compared with DMSO-treated mice was found, in both CD34+ HSPCs and B cells (FIG. 19g-h and FIG. 20b). Remarkably, when telomere length was measured in bone marrow cells of xenotransplanted mice by flow cytometry-fluorescence in situ hybridization, PAPD5 inhibition by DHQ-1 reversed telomere shortening in PARN-deficient human CD45+ cells was found (FIG. 19i and FIG. 20c). DHQ-1 treatment did not alter overall human hematopoietic cell engraftment or differentiation capacity (FIG. 20d-e). Taken together, these data demonstrate in vitro and in vivo manipulation of telomerase RNA maturation and telomere length in a disease-relevant, human primary stem cell compartment using small molecule PAPD5 inhibitors.

### REFERENCES

1. H. Mueller et al., PAPD5/7 Are Host Factors That Are Required for Hepatitis B Virus RNA Stabilization. Hepatology 69, 1398-1411 (2019).

2. Y. Wu et al., Highly efficient therapeutic gene editing of human hematopoietic stem cells. Nat Med 25, 776-783 (2019).

3. B. E. McIntosh et al., Nonirradiated NOD,B6.SCID Il2rgamma−/− Kit(W41/W41) (NBSGW) mice support multilineage engraftment of human hematopoietic cells. Stem Cell Reports 4, 171-180 (2015).

4. H. Mueller et al., A novel orally available small molecule that inhibits hepatitis B virus expression. J Hepatol 68, 412-420 (2018).

### Example 3

### DHQ Compounds are Selective Inhibitors of PAPD5

The compounds described herein are specific and selective inhibitors of PAPD5. For example, the PAPD5 inhibitors of the present disclosure do not inhibit PARN or other polynucleotide polymerases.

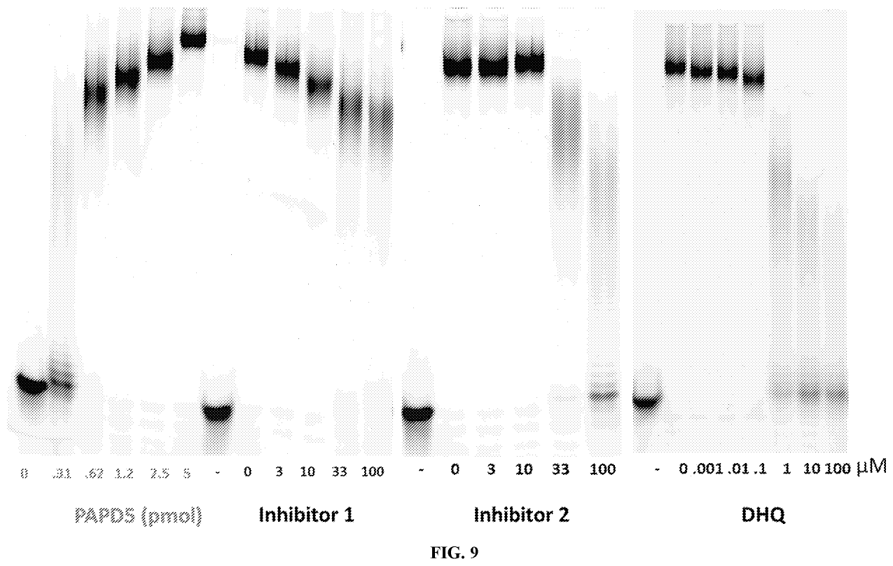

Referring to FIGS. 9-12:

Inhibitor 2 refers to compound 1-(1,3-benzodioxo1-5-ylmethyl)-5-oxopyrrolidine-3-carboxylic acid having formula:

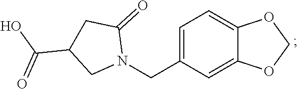 [SMILES: O=C(O)C1CC(=O)N(Cc2ccc3c(c2)OCO3)C1]

Inhibitor 1 refers to compound 2-[[3-ethoxycarbonyl-6-(trifluoromethoxy)quinolin-4-yl]amino]benzoic acid having formula:

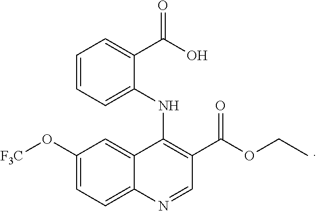 [SMILES: CCOC(=O)c1cnc2ccc(OC(F)(F)F)cc2c1Nc1ccccc1C(=O)O]

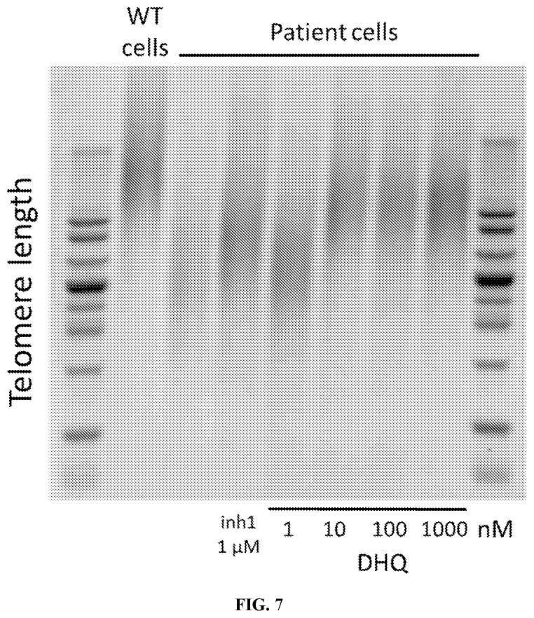

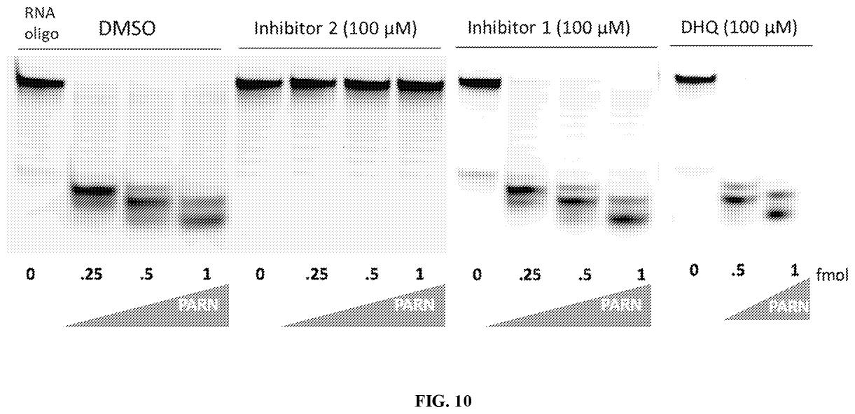

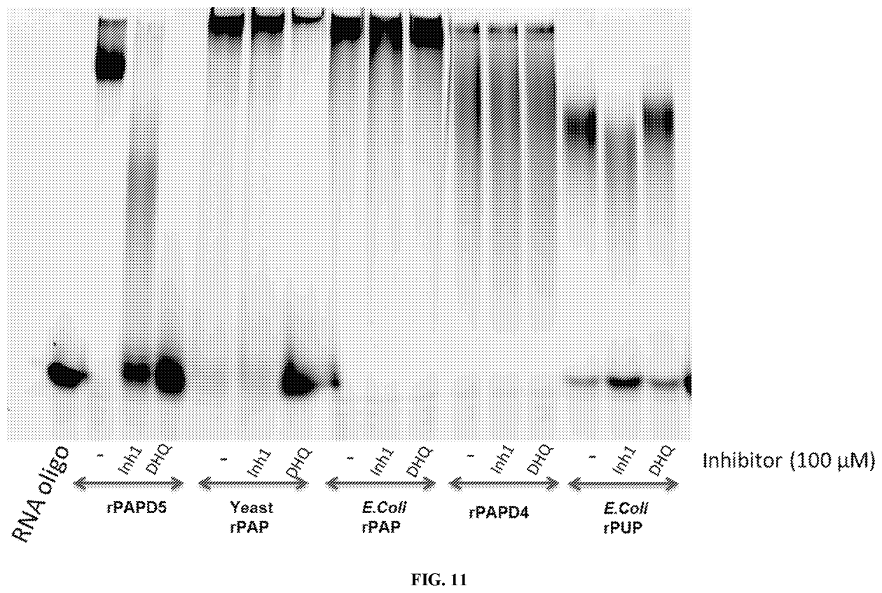

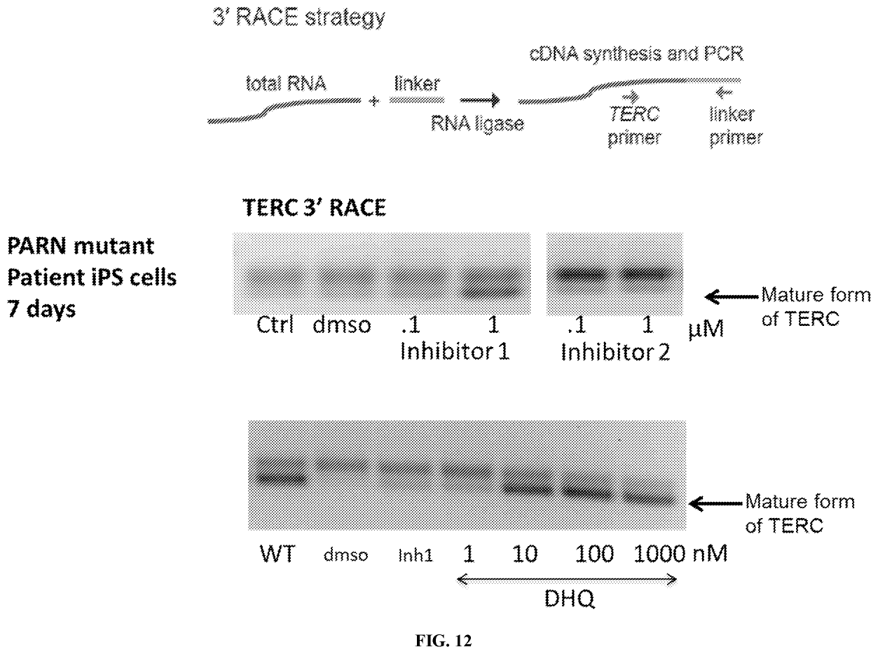

FIG. 9 shows rPAPD5 inhibition in vitro by compounds inhibitor 2, inhibitor 1, and DHQ (DHQ-1). FIGS. 10 and 11 show that compound inhibitor 2 does inhibit PARN exonuclease inhibitor 1 and DHQ do not inhibit PARN and do not inhibit multiple poly-nucleotide polymerases. As shown in FIG. 12, inhibitor 1 and DHQ restore telomerase RNA (TERC) end processing whereas compound inhibitor 2 does not. As shown in FIG. 7, like Inhibitor 1, DHQ restores telomere length in DC patient iPS cells.

### Example 4

### Exemplified Compounds are Inhibitors of PAPD5, Restore TERC End Processing, and Restore Telomere Length

| Com - | |
|---|---|
| pound | |
| No. | structure |
| DHQ-1 | 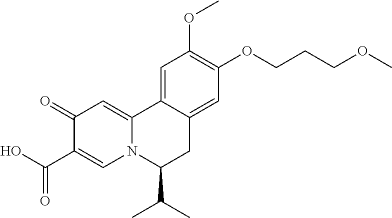 [SMILES: COCCCOc1cc2c(cc1OC)-c1cc(=O)c(C(=O)O)cn1[C@H](C(C)C)C2] |
| 18C | 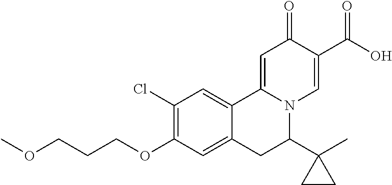 [SMILES: COCCCOc1cc2c(cc1Cl)-c1cc(=O)c(C(=O)O)cn1C(C1(C)CC1)C2] |
| 19C | 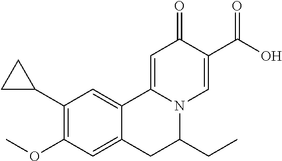 [SMILES: CCC1Cc2cc(OC)c(C3CC3)cc2-c2cc(=O)c(C(=O)O)cn21] |
| 1C | 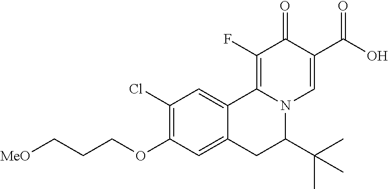 [SMILES: COCCCOc1cc2c(cc1Cl)-c1c(F)c(=O)c(C(=O)O)cn1C(C(C)(C)C)C2] |
| 20C | 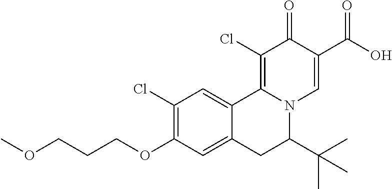 [SMILES: COCCCOc1cc2c(cc1Cl)-c1c(Cl)c(=O)c(C(=O)O)cn1C(C(C)(C)C)C2] |
| 2C | 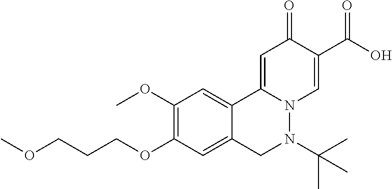 [SMILES: COCCCOc1cc2c(cc1OC)-c1cc(=O)c(C(=O)O)cn1N(C(C)(C)C)C2] |
| 3C | 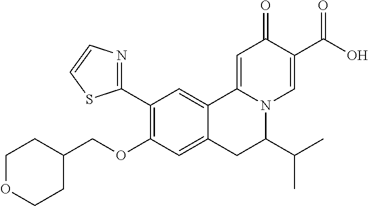 [SMILES: CC(C)C1Cc2cc(OCC3CCOCC3)c(-c3nccs3)cc2-c2cc(=O)c(C(=O)O)cn21] |
| 4C | 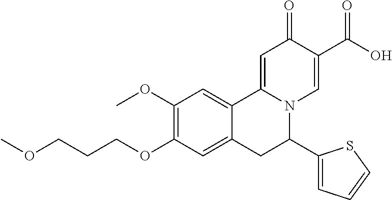 [SMILES: COCCCOc1cc2c(cc1OC)-c1cc(=O)c(C(=O)O)cn1C(c1cccs1)C2] |
| 5C | 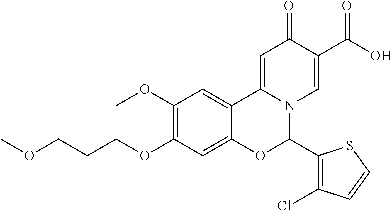 [SMILES: COCCCOc1cc2c(cc1OC)-c1cc(=O)c(C(=O)O)cn1C(c1sccc1Cl)O2] |
| 22C | 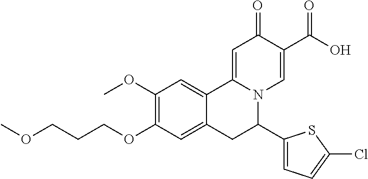 [SMILES: COCCCOc1cc2c(cc1OC)-c1cc(=O)c(C(=O)O)cn1C(c1ccc(Cl)s1)C2] |
| 9C | 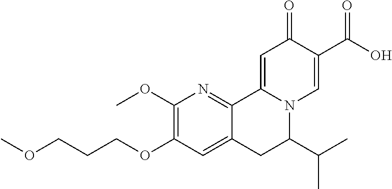 [SMILES: COCCCOc1cc2c(nc1OC)-c1cc(=O)c(C(=O)O)cn1C(C(C)C)C2] |
| 10C | 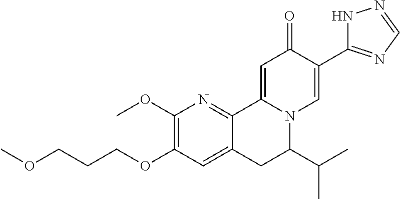 [SMILES: COCCCOc1cc2c(nc1OC)-c1cc(=O)c(-c3ncn[nH]3)cn1C(C(C)C)C2] |
| 7C-1 | 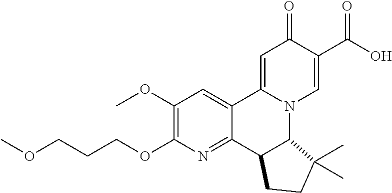 |
| 7C-2 | 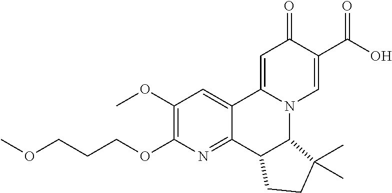 |
| 12C | 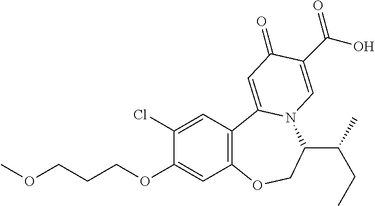 |

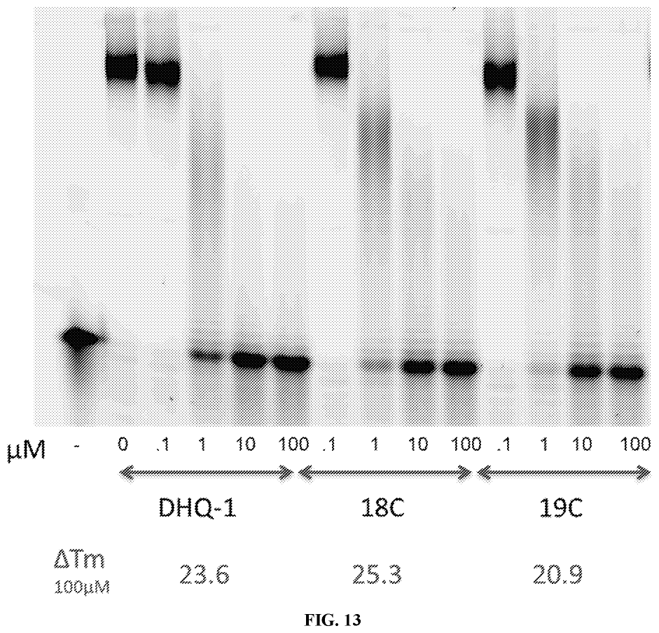

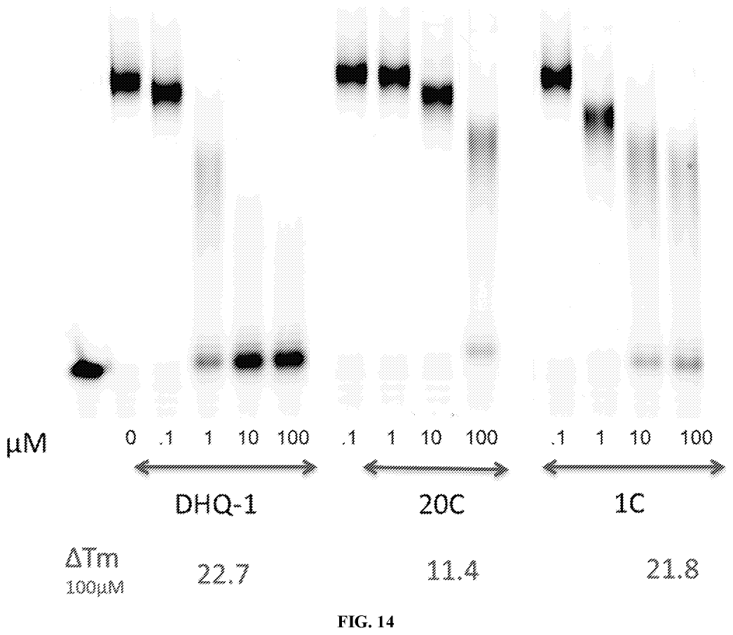

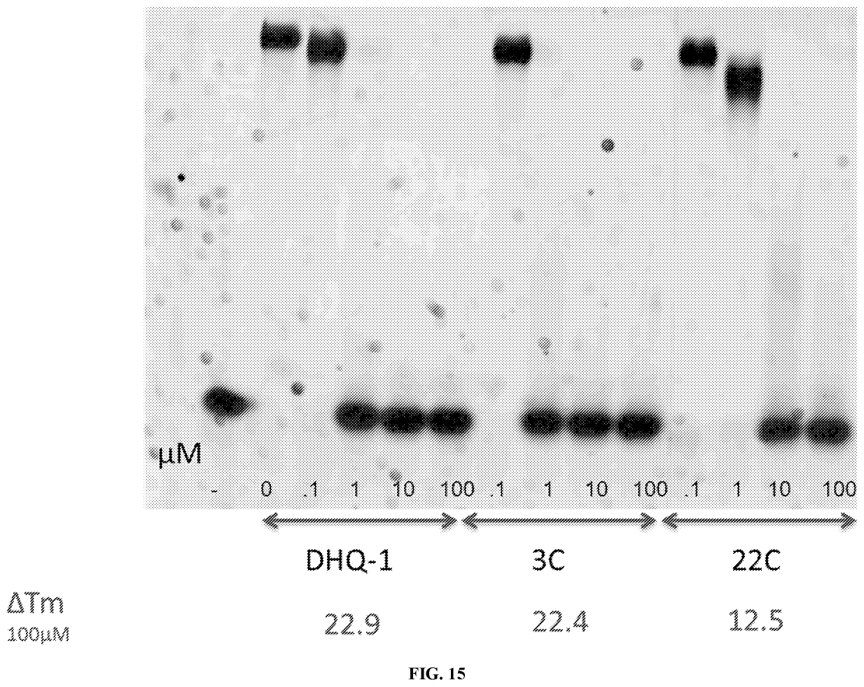

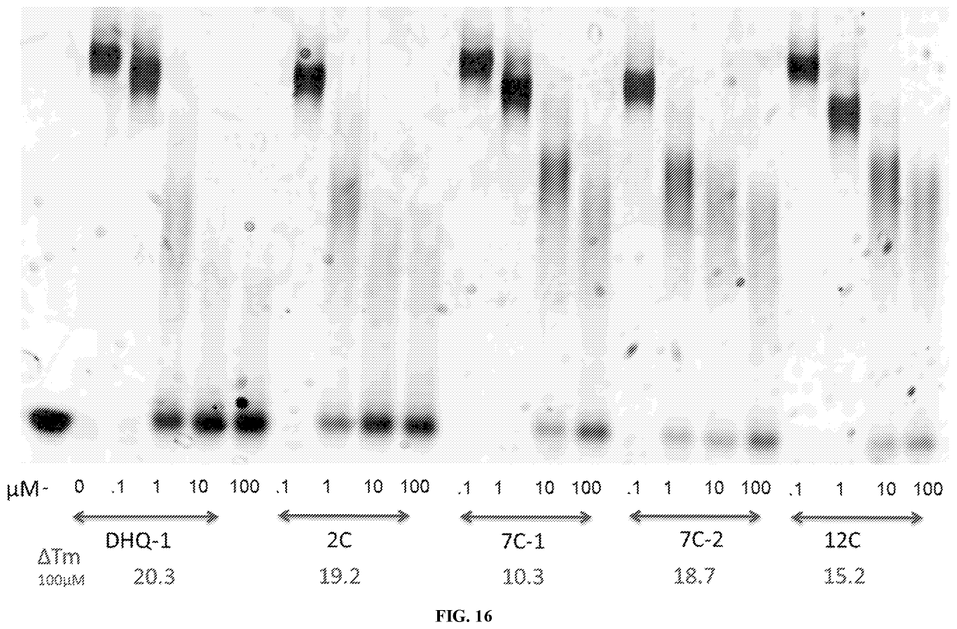

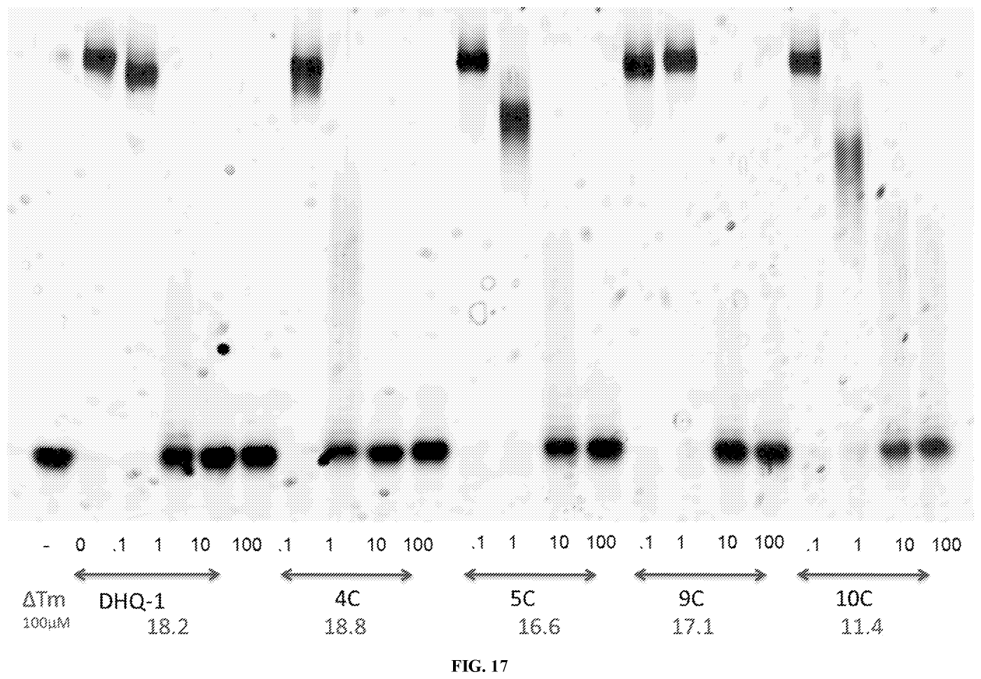

FIGS. 13-17 show activity of compounds DHQ-1, 20C, 1C, 3C, 22C, 2C, 7C-1, 7C-2, 12C, 4C, 5C, 9C, 10C, 18C, and 19C in RNA oligo-adenylation assay.

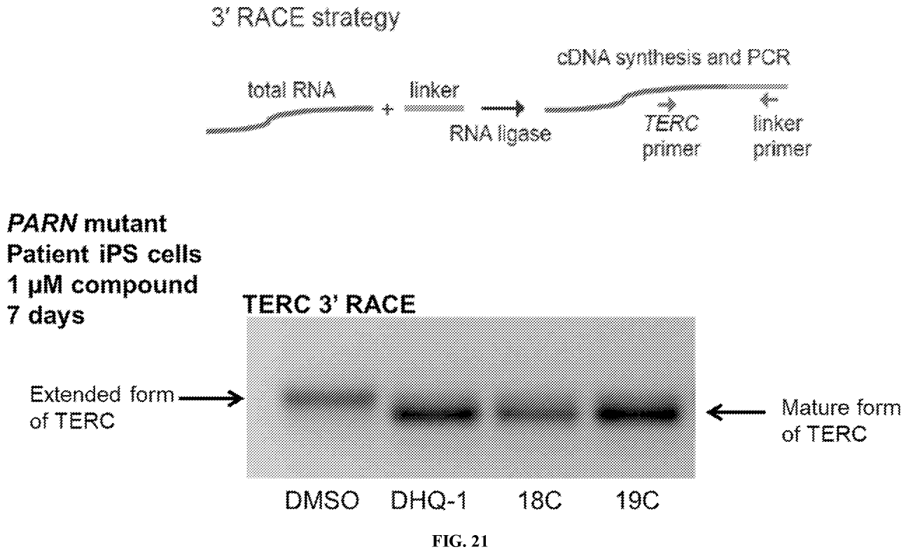

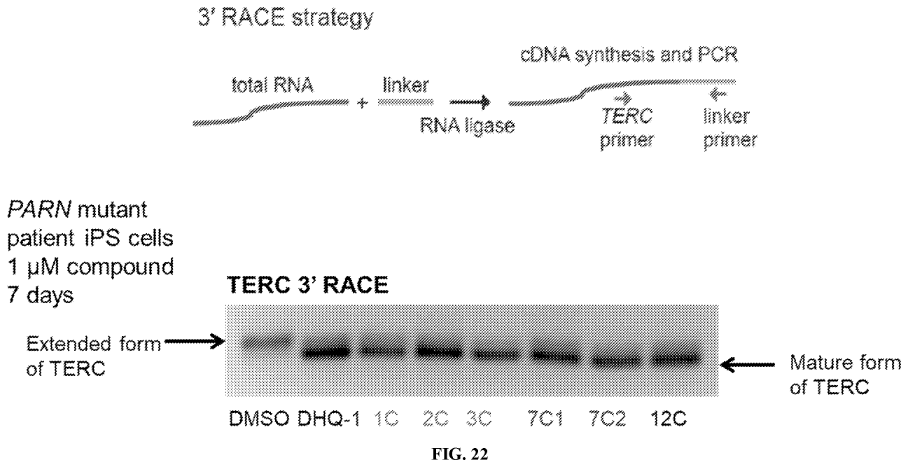

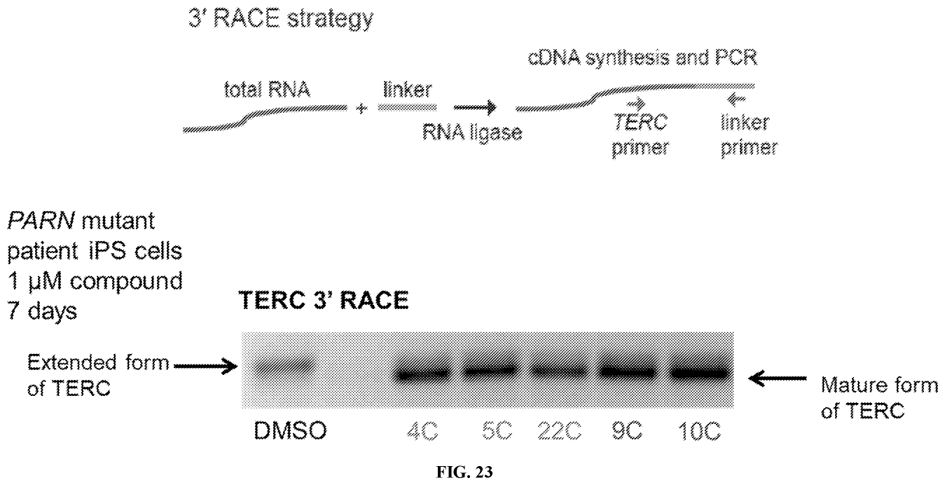

FIGS. 21-23 show that DHQ-1 and compounds 1C, 2C, 3C, 7C-1, 7C-2, 12C, 18C, 19C, 4C, 5C, 22C, 9C, and 10C restore telomerase RNA (TERC) end processing

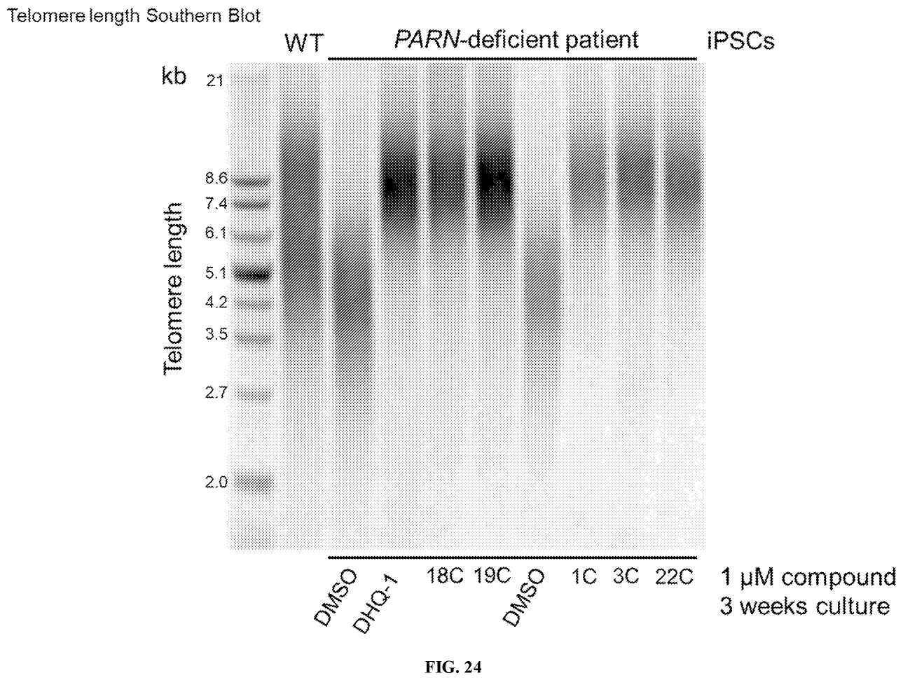

FIG. 24 shows that compound DHQ-1 and compounds 18C, 19C, 1C, 3C, and 22C elongate telomeres.

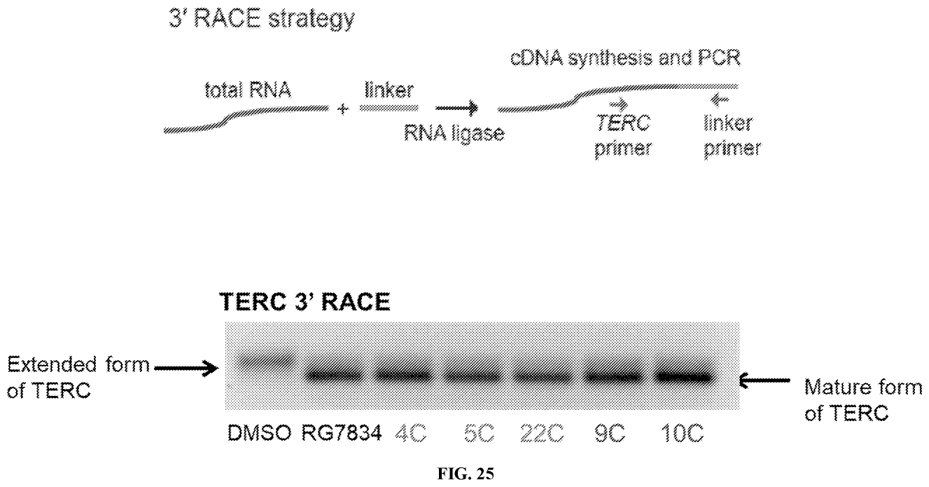

FIG. 25 shows that DHQ-1 and compounds 4C, 5C, 22C, 9C, and 10C restore telomerase RNA (TERC) end processing.

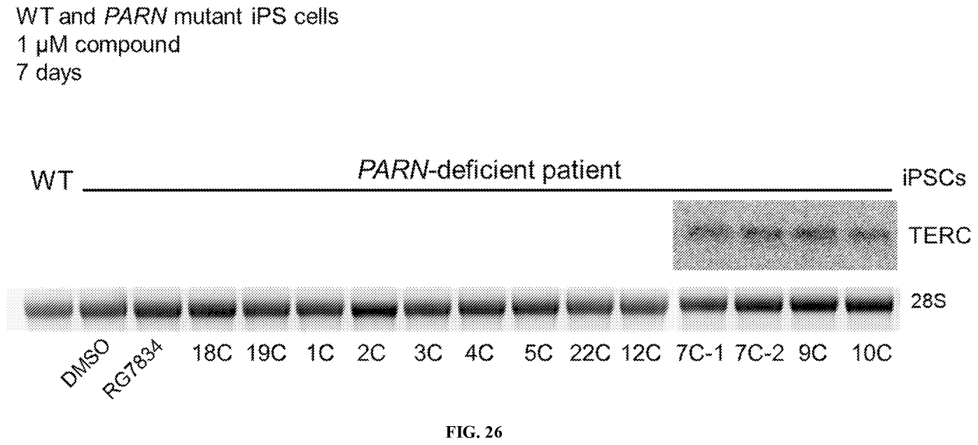

FIG. 26 shows that DHQ-1 and compounds 18C, 19C, 1C, 2C, 3C, 4C, 5C, 22C, 12C, 7C-1, 7C-2, 9C, and 10C restore telomerase RNA (TERC) levels

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A method of treating a disease or condition selected from
(1) a disorder associated with telomere or telomerase dysfunction;
(2) a disorder associated with aging;
(3) a pre-leukemic or pre-cancerous condition; and
(4) neurodevelopmental disorder
in a subject in need thereof; the method comprising administering to the subject in need thereof a therapeutically effective amount of a compound of Formula (I): 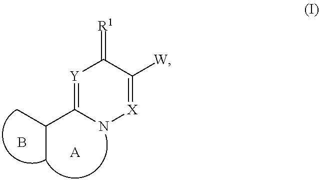
or a pharmaceutically acceptable salt thereof, wherein:
R¹ is selected from O, S, N—OH, N—C₁₋₃ alkoxy, N—NH₂, and N—CN;
W is selected from C(O)OR^{a1}, C(O)NR^{c1}R^{d1}, C(O)NR^{c1}S(O)₂R^{b1}, C(O)NR^{c1}OR^{a1}, NR^{c1}C(O)OR^{a1}, NR^{c1}C(O)NR^{c1}R^{d1}, NR^{c1}C(O)R^{b1}, OR^{a1}, NR^{c1}R^{d1}, NR^{c1}S(O)₂R^{b1}, B(OH)₂, P(═O)(OR^{a1})₂, halo, CN, Cy, and a carboxylic acid bioisostere;
or R¹ and W together with the carbon atoms to which they are attached from a monocyclic 4-7 membered heterocycloalkyl ring or a monocyclic 5-6 membered heteroaryl ring, each of which is optionally substituted with 1, 2, or 3 substituents independently selected from R^{Cy};
X is selected from N and CR²;
Y is selected from N and CR³;
R² is selected from H, Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
R³ is selected from H, Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.
ring A, together with N and other atom or atoms that ring A shares with ring B, is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{A};
ring B, together with the atom or atoms that ring B shares with ring A, is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{B};
each R^{A} is independently selected from H, Cy, halo, CN, NO₂, OR^{a1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
or any two R^{A} groups together with the atom or atoms to which they are attached form ring C, which is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{C};
each R^{B} is independently selected from H, Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
or any two R^{B} groups together with the atom or atoms to which they are attached form ring D, which is selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{D};
or any two R^{A} and R^{B} groups together with the atoms to which they are attached form a ring selected from a monocyclic C₃₋₇ cycloalkyl ring, a monocyclic 4-7 membered heterocycloalkyl ring, and a monocyclic 5-6 membered heteroaryl ring, each of which is optionally substituted with 1, 2, 3, 4, or 5 substituents independently selected from R^{Cy};
each R^{C} and R^{D} are independently selected from H, Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, S(O)₂NR^{c1}R^{d1}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
or any two R^{C} groups together with the atom or atoms to which they are attached form a ring selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{Cy};
or any two R^{D} groups together with the atom or atoms to which they are attached form a ring selected from a monocyclic C₃₋₇ cycloalkyl ring, a monocyclic 4-7 membered heterocycloalkyl ring, a phenyl ring, and a monocyclic 5-6 membered heteroaryl ring, each of which is optionally substituted with 1, 2, 3, 4, or 5 substituents independently selected from R^{Cy};
Cy is selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{Cy};
each R^{Cy} is independently selected from H, halo, CN, NO₂, OR^{a1}, C(O)R^{b1}, wherein said C₁₋₆ alkyl, C₂₋₆ alkenyl, and C₂₋₆ alkynyl are each optionally substituted with 1, 2, or 3 substituents independently selected from halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}NR^{c1}C(O)OR^{a1}, NR^{c1}S(O)₂R^{b1}, S(O)₂R^{b1}, and S(O)₂NR^{c1}R^{d1}.
each R^{a1}, R^{b1}, R^{c1}, and R^{d1} is independently selected from Cy¹, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from Cy¹, halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2},
or any R^{c1} and R^{d1} together with the N atom to which they are attached form a 4-7 membered heterocycloalkyl, which is optionally substituted with 1, 2, or 3 substituents independently selected from R^{g};
Cy¹ is selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{Cy1};
each R^{Cy1} is independently selected from H, halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, S(O)₂NR^{c2}R^{d2}, C₁₋₆ alkyl, C₁₋₆ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, N^{c2}C(O)OR^{a2}, NR^{c2}S(O)₂R^{b2}, S(O)₂R^{b2}, and S(O)₂NR^{c2}R^{d2},
each R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c2} and R^{d2} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

2. The method of claim 1, wherein R¹ is O.

3. The method of claim 1, wherein W is C(O)OH or a carboxylic acid bioisostere.

4. The method of claim 3, wherein the carboxylic acid bioisostere has any one of the following formulae:

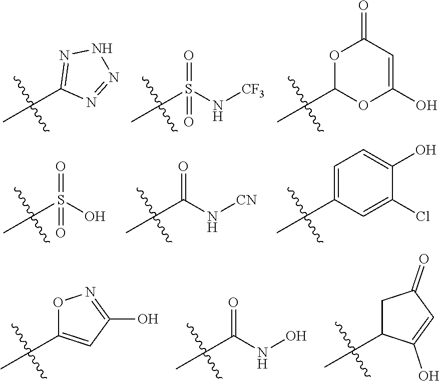

5. The method of claim 1, wherein X is CR² and Y is CR³.

6. The method of claim 5, wherein R² is H, and R³ is selected from H and halo.

7. The method of claim 1, wherein ring A is a monocyclic 4-7 membered heterocycloalkyl ring.

8. The method of claim 1, wherein ring B is selected from a monocyclic 4-7 membered heterocycloalkyl ring, a phenyl ring, and a monocyclic 5-6 membered heteroaryl ring.

9. The method of claim 1, wherein R^{A} is selected from Cy and C₁₋₆ alkyl, wherein said C₁₋₆ alkyl is optionally substituted with 1, 2, or 3 substituents independently selected from halo and OR^{a1}.

10. The method of claim 1, wherein R^{B} is selected from Cy, halo, OR^{a1}, C(O)R^{b1}, and C₁₋₆ alkyl or C₂₋₆ alkynyl, each of which is optionally substituted with Cy¹, OR^{a2}, and S(O)₂R^{b2}.

11. The method of claim 1, wherein R^{Cy} is selected from halo, CN, NO₂, OH, amino, C₁₋₆ alkyl, C₁₋₆ haloalkyl, C₁₋₆ alkoxy, and C₁₋₆ haloalkoxy.

12. The method of claim 1, wherein each R^{a1}, R^{b1}, R^{c1}, and R^{d1} is independently selected from Cy¹, C₁₋₆ alkyl, and C₂₋₆ alkynyl, wherein said C₁₋₆ alkyl and C₂₋₆ alkynyl are each optionally substituted with 1, 2, or 3 substituents independently selected from Cy¹, halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}.

13. The method of claim 1, wherein the compound of Formula (I) is selected from any one of the following formulae: 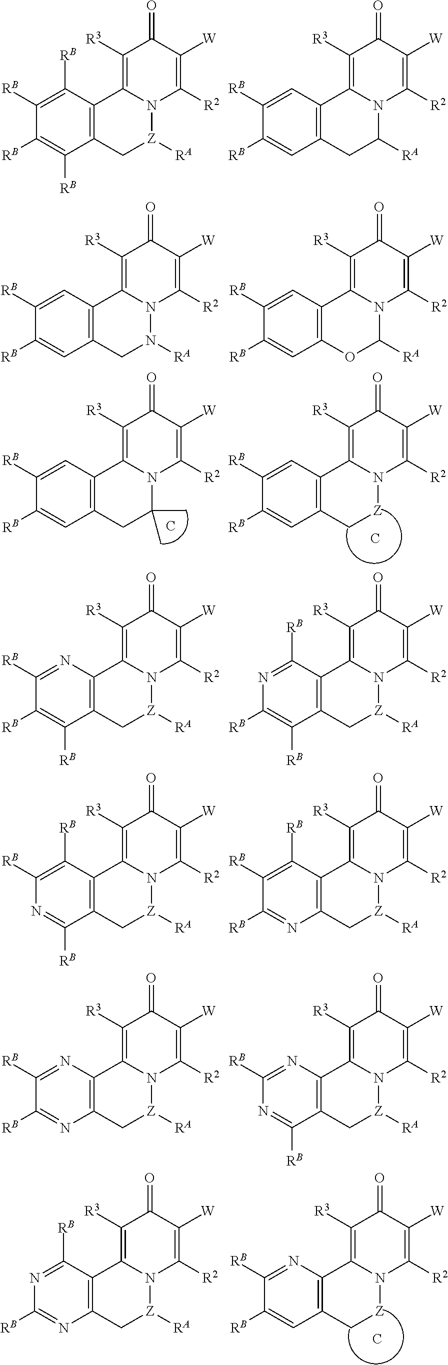 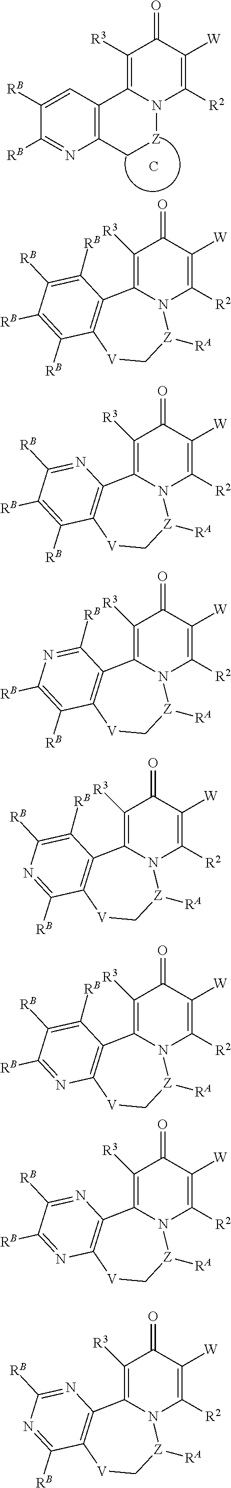 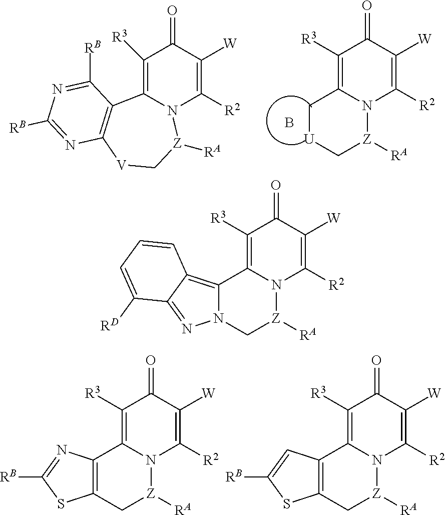
or a pharmaceutically acceptable salt thereof, wherein:
Z is selected from N and CR^{A};
V is selected from O, NR^{A}, and C(R^{A})₂; and
U is selected from N, C, and CR^{A}.

14. The method of claim 13, wherein Z is N.

15. The method of claim 13, wherein Z is CH.

16. The method of claim 13, wherein V is O.

17. The method of claim 13, wherein R^{D} is OR^{a1}.

18. The method of claim 1, wherein the compound of Formula (I) is selected from any one of the compounds disclosed in: WO2018022282, US20180170925, WO2018219356, US20170342068, WO2017205115, US20160122344, WO2015173164, WO2016128335, WO2017013046, WO2017017043, WO2017102648, WO2018161960, WO2018154466, WO2018019297, CN108727378, US20180251460, WO2018144605, WO2017140821, U.S. Ser. No. 10/093,673, CN106928245, WO2017017042, WO2018047109, WO2018085619, WO2018214875, WO2018198079, and US20180312507, or a pharmaceutically acceptable salt thereof.

19. The method of claim 1, wherein the compound of Formula (I) is selected from any one of the following compounds: 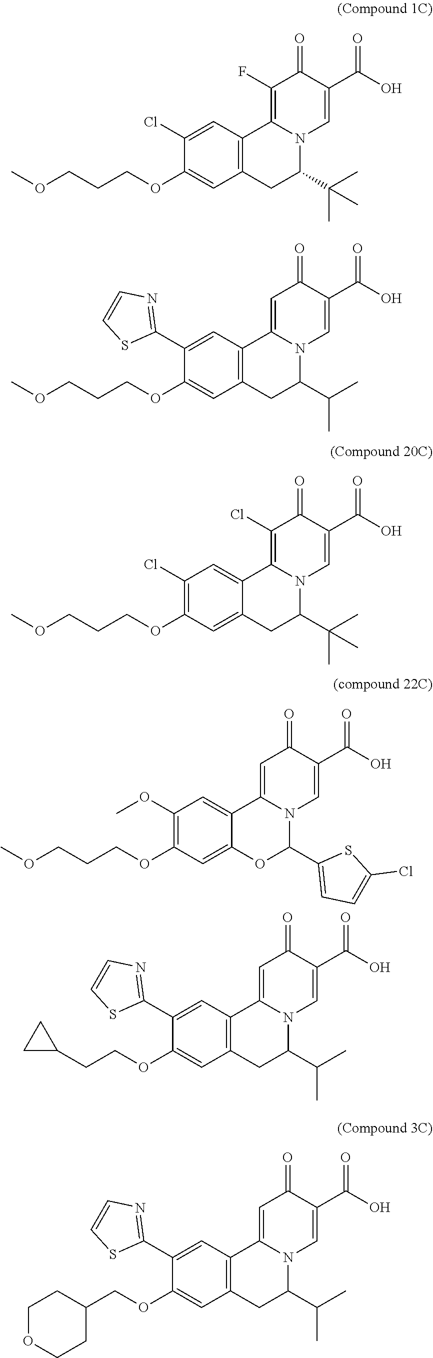 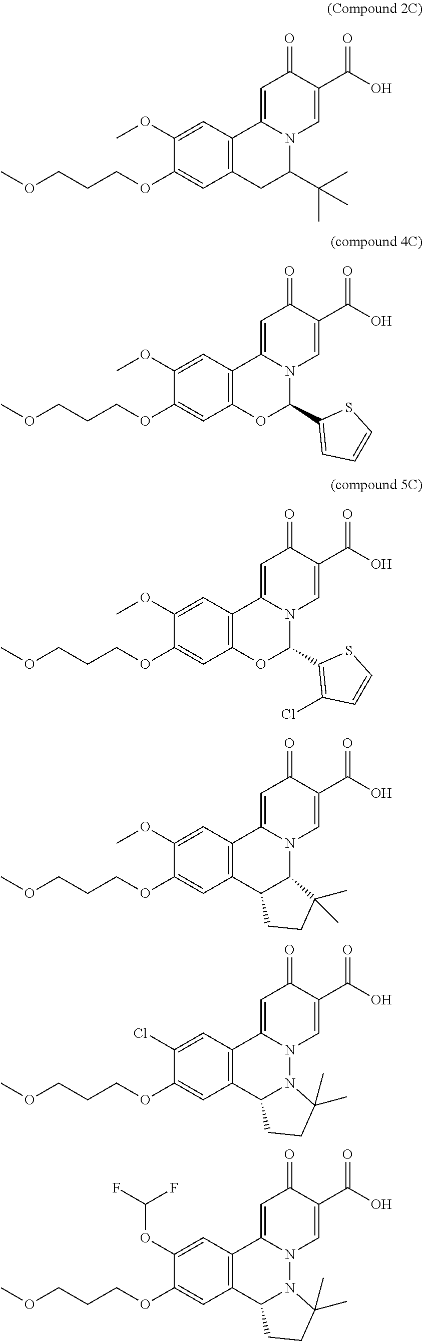 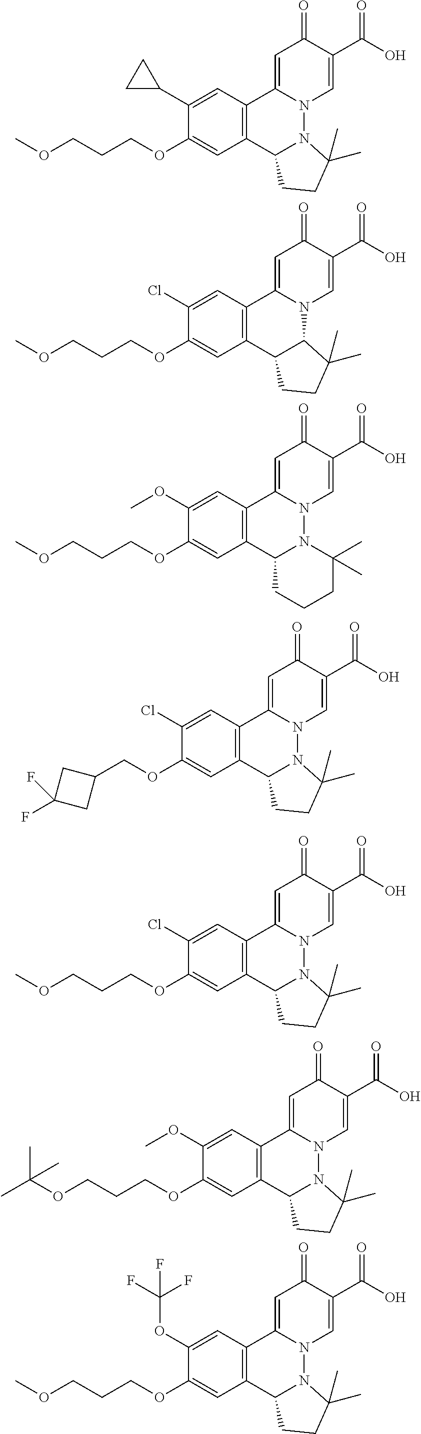 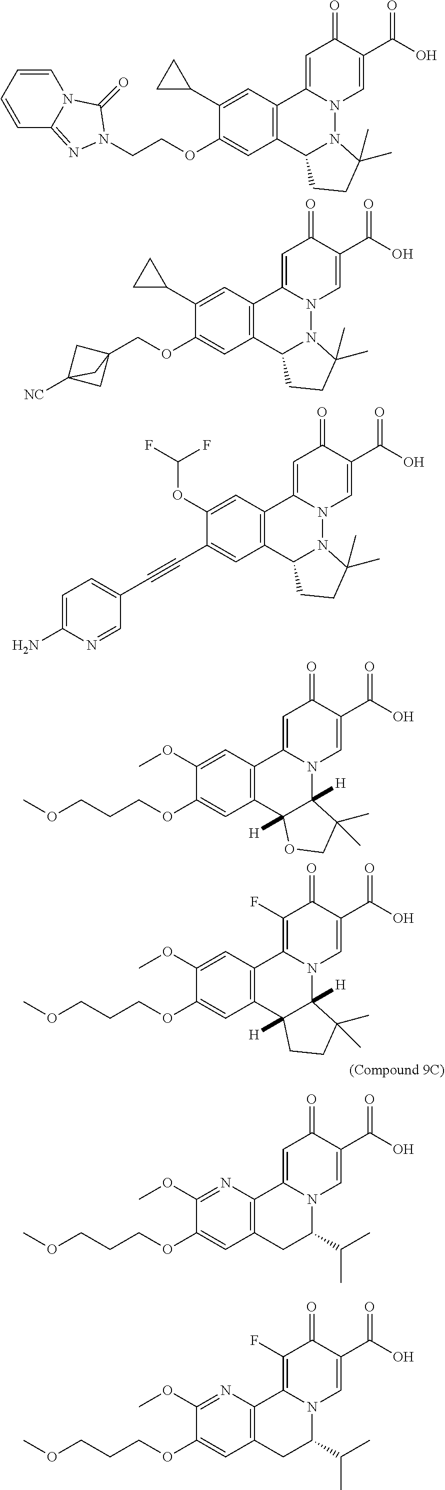 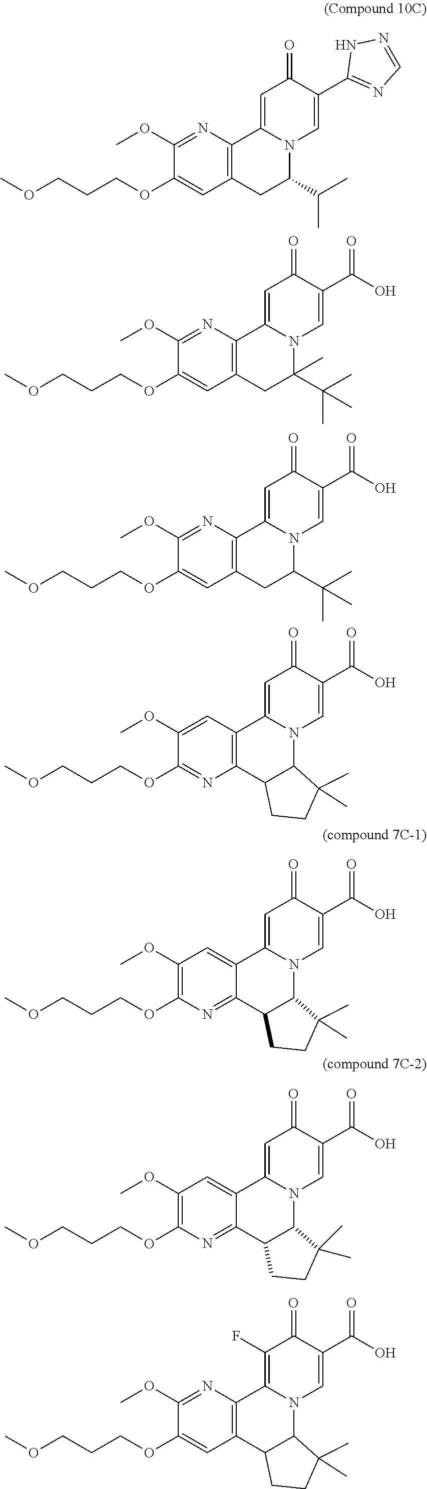 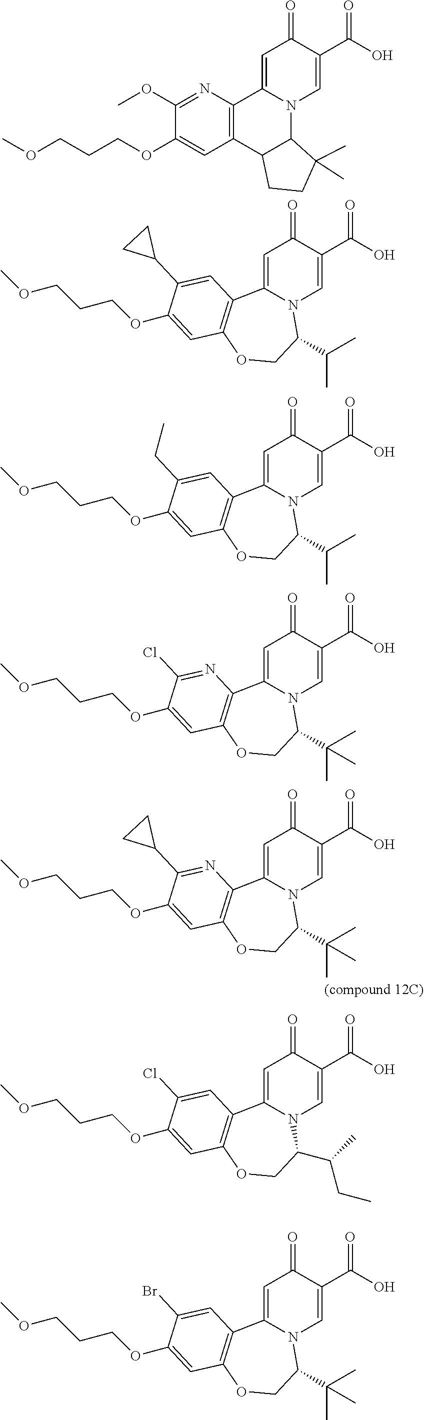 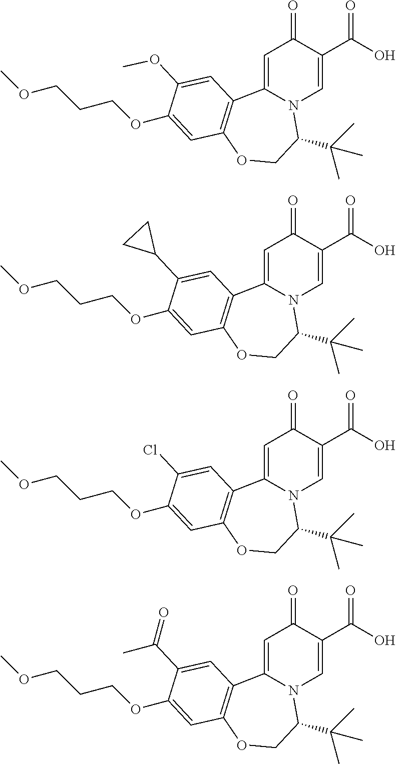
or a pharmaceutically acceptable salt thereof.

20. The method of claim 1, wherein the compound of Formula (I) is selected from any one of the following compounds: 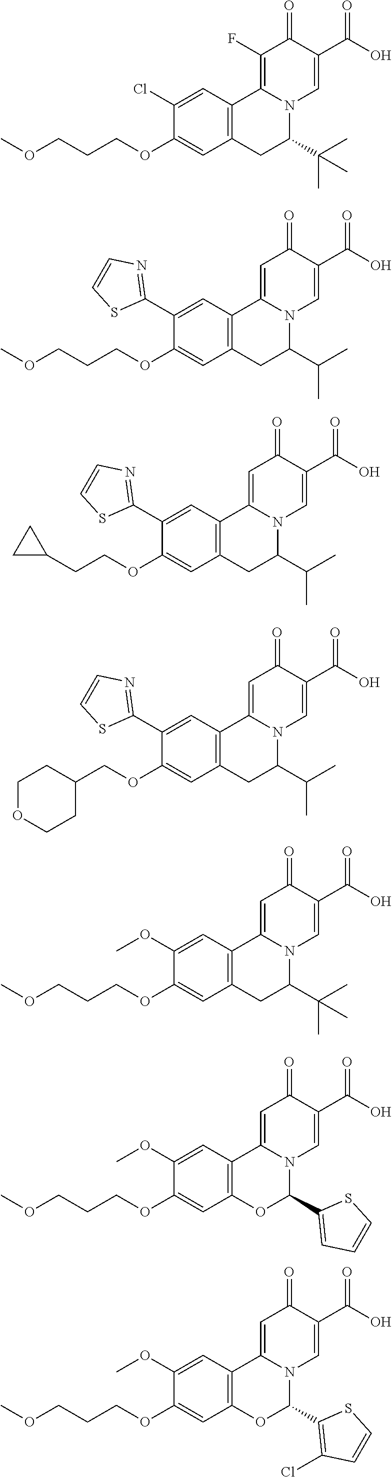 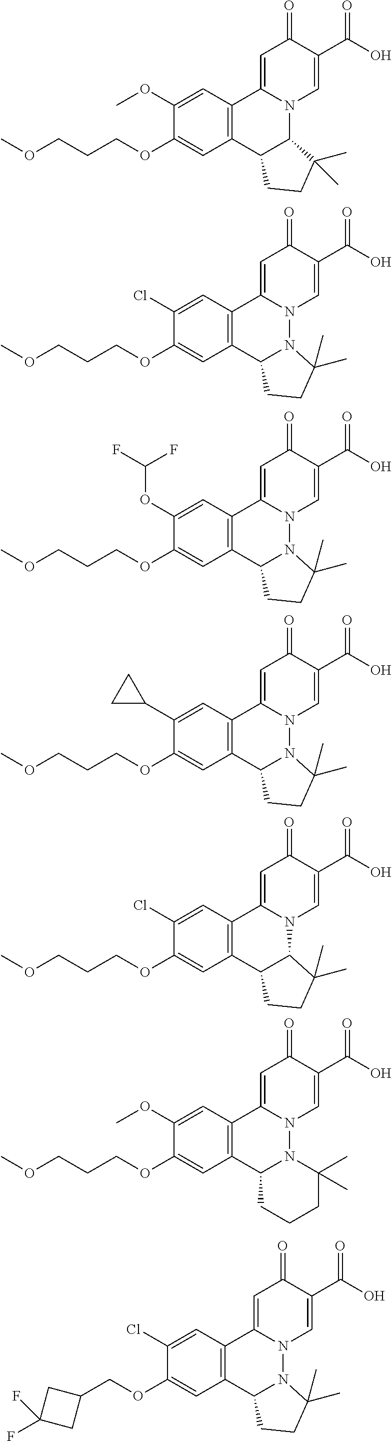 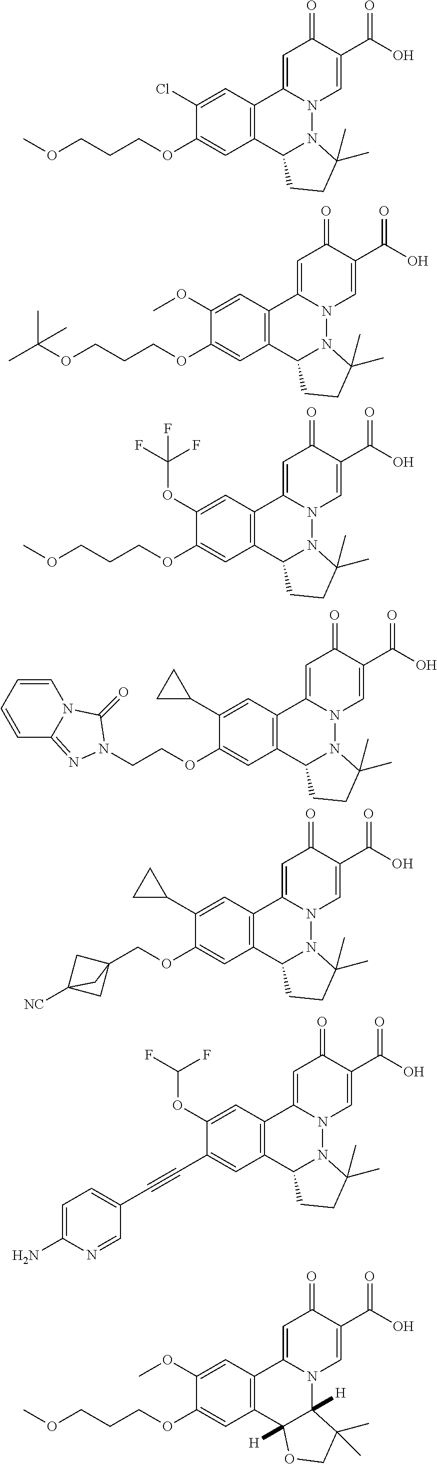 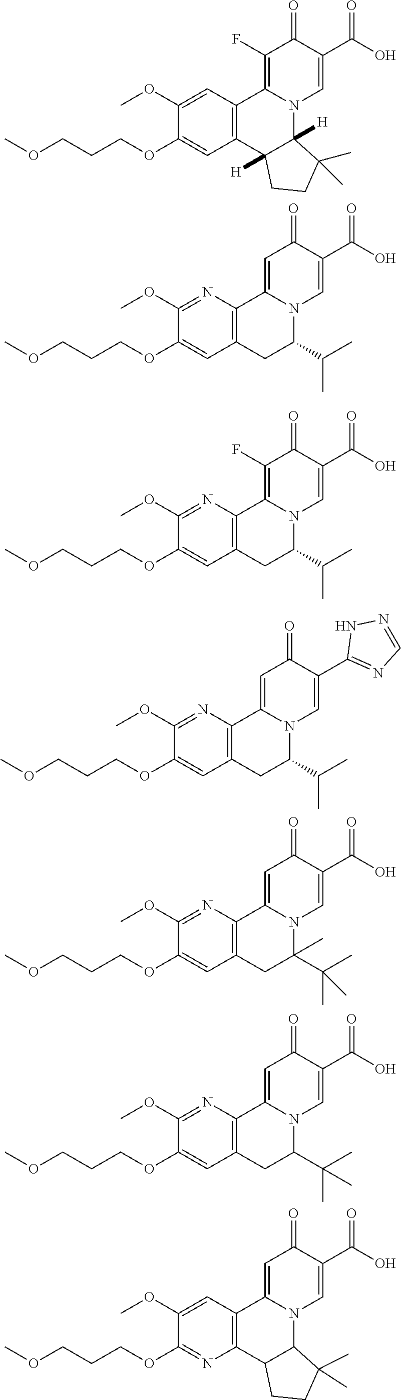 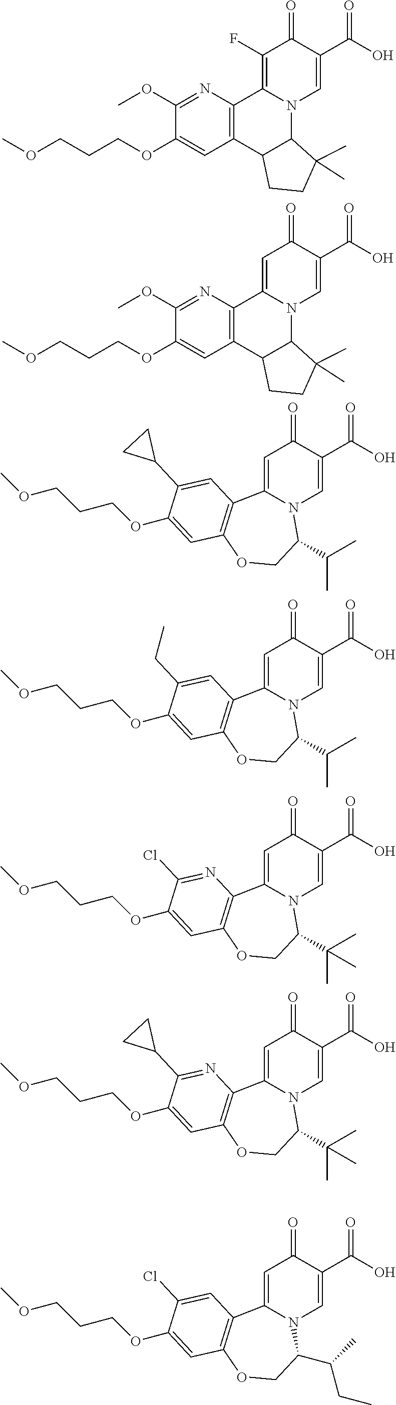 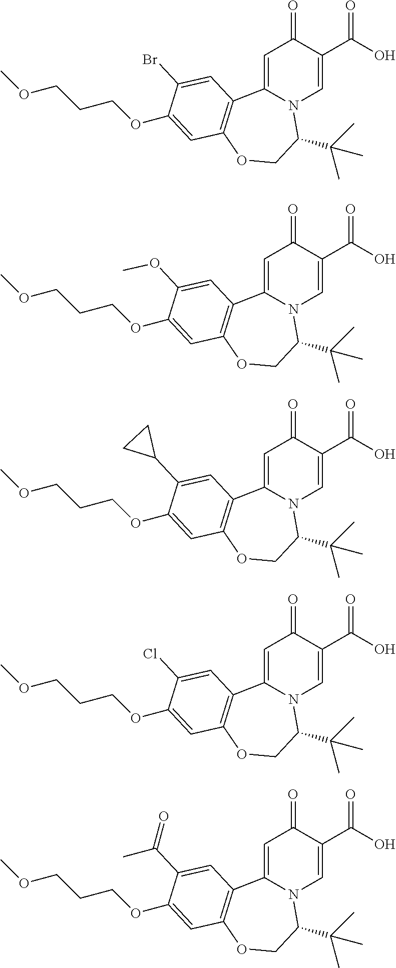
or a pharmaceutically acceptable salt thereof.

21. The method of claim 1, wherein the compound of Formula (I) is selected from any one of the following compounds: 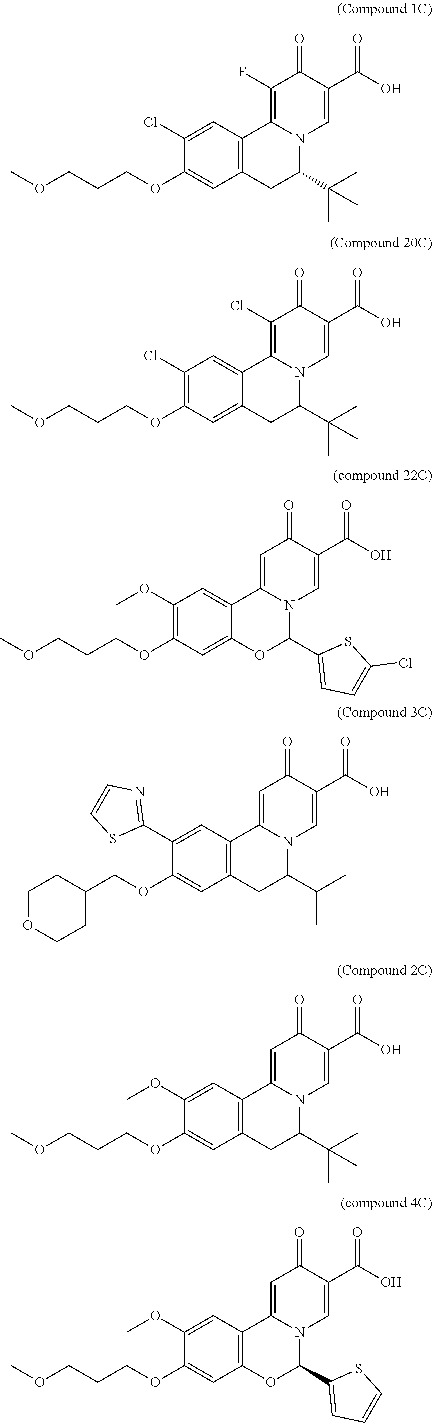 [SMILES: COCCCOc1cc2c(cc1Cl)-c1c(F)c(=O)c(C(=O)O)cn1[C@H](C(C)(C)C)C2 ; COCCCOc1cc2c(cc1Cl)-c1c(Cl)c(=O)c(C(=O)O)cn1C(C(C)(C)C)C2 ; COCCCOc1cc2c(cc1OC)-c1cc(=O)c(C(=O)O)cn1C(c1ccc(Cl)s1)O2 ; CC(C)C1Cc2cc(OCC3CCOCC3)c(-c3nccs3)cc2-c2cc(=O)c(C(=O)O)cn21 ; COCCCOc1cc2c(cc1OC)-c1cc(=O)c(C(=O)O)cn1C(C(C)(C)C)C2 ; COCCCOc1cc2c(cc1OC)-c1cc(=O)c(C(=O)O)cn1[C@@H](c1cccs1)O2] 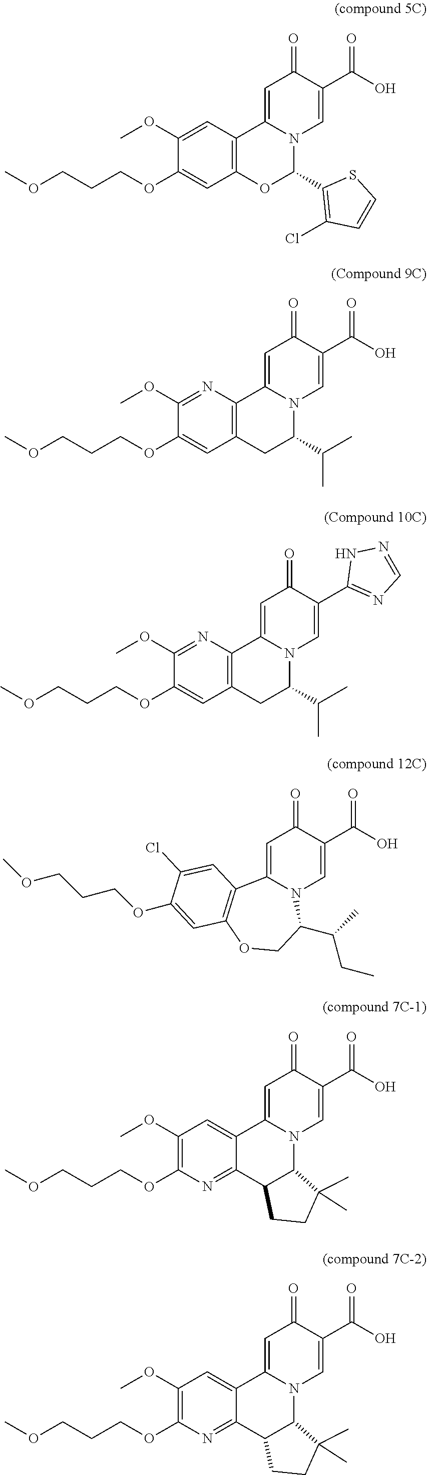 [SMILES: COCCCOc1cc2c(cc1OC)-c1cc(=O)c(C(=O)O)cn1[C@H](c1sccc1Cl)O2 ; COCCCOc1cc2c(nc1OC)-c1cc(=O)c(C(=O)O)cn1[C@H](C(C)C)C2 ; COCCCOc1cc2c(nc1OC)-c1cc(=O)c(-c3ncn[nH]3)cn1[C@H](C(C)C)C2 ; CC[C@@H](C)[C@@H]1COc2cc(OCCCOC)c(Cl)cc2-c2cc(=O)c(C(=O)O)cn21 ; COCCCOc1nc2c(cc1OC)-c1cc(=O)c(C(=O)O)cn1[C@H]1[C@H]2CCC1(C)C ; COCCCOc1nc2c(cc1OC)-c1cc(=O)c(C(=O)O)cn1[C@H]1[C@@H]2CCC1(C)C]
or a pharmaceutically acceptable salt thereof.

22. A method of treating a disease or condition selected from
(1) a disorder associated with telomere or telomerase dysfunction;
(2) a disorder associated with aging;
(3) a pre-leukemic or pre-cancerous condition; and
(4) neurodevelopmental disorder
in a subject in need thereof; the method comprising administering to the subject in need thereof a therapeutically effective amount of a compound of Formula (II): 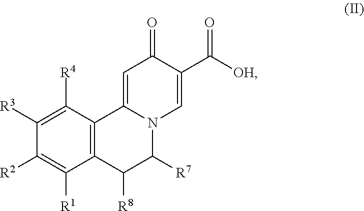
or a pharmaceutically acceptable salt thereof, wherein:
R¹ is selected from H, C₁₋₆ alkyl, halo, CN, and OR^{a1};
R² is selected from H, C₁₋₆ alkyl, C₁₋₄ haloalkyl, Cy¹, halo, CN, OR^{a1}, and NR^{c1}R^{d1};
R³ is selected from H, C₁₋₆ alkyl, C₁₋₄ haloalkyl, halo, and OR^{a1};
R⁴ is selected from H, C₁₋₆ alkyl, halo, OR^{a1}, and NR^{c1}R^{d1},
R⁷ is selected from H, C₁₋₆ alkyl, C₁₋₄ haloalkyl, Cy¹, and halo; wherein said C₁₋₆ alkyl is optionally substituted with Cy¹;
R⁸ is selected from H and C₁₋₆ alkyl;
R^{a1}, R^{c1}, and R^{d1} are each independently selected from H, C₁₋₆ alkyl, C₂₋₆ alkenyl, and C₂₋₆ alkynyl; wherein said C₁₋₆ alkyl, C₂₋₆ alkenyl, and C₂₋₆ alkynyl are each optionally substituted with 1, 2, or 3 substituents independently selected from Cy³, halo, CN, OR^{a3}, C(O)R^{b3}, C(O)OR^{a3}, NR^{c3}R^{d3}, NR^{c3}S(O)₂R^{b3}, S(O)R^{b3}, and S(O)₂R^{b3};
R^{a3}, R^{c3}, and R^{d3} are each independently selected from H, C₁₋₆ alkyl, C(O)R^{b4}, and C(O)OR^{a4}; wherein said C₁₋₆ alkyl is optionally substituted with OR^{a4} or NR^{c4}R^{d4};
each R^{b3} is independently selected from C₁₋₆ alkyl and 4-12 membered heterocycloalkyl;
each Cy¹ is independently selected from C₆₋₁₀ aryl, C₃₋₁₀ cycloalkyl, 5-10 membered heteroaryl, and 4-12 membered heterocycloalkyl, each of which is optionally substituted with 1, 2, or 3 substituents independently selected from R^{Cy1};
each Cy³ is independently selected from C₆₋₁₀ aryl, C₃₋₁₀ cycloalkyl, 5-10 membered heteroaryl, and 4-12 membered heterocycloalkyl, each of which is optionally substituted with 1, 2, or 3 substituents independently selected from R^{Cy3};
R^{Cy1} and R^{Cy3} are each independently selected from halo, C₁₋₄ alkyl, CN, and C(O)OR^{a4},
R^{a4}, R^{c4}, and R^{d4} are each independently selected from H and C₁₋₆ alkyl; and
each R^{b4} is C₁₋₆ alkyl.

23. The method of claim 22, wherein:
R⁴ is hydrogen, fluoro, chloro, bromo, methyl, methylamino, methoxy or ethoxy;
R³ is hydrogen, fluoro, chloro, bromo, methyl, ethyl, trifluoromethyl, methoxy, ethoxy, propoxy, trifluoromethoxy, cyano, cyclopropyl, hydroxy or phenylmethyl-O—;
R² is hydrogen, bromo, methyl, propyl, trifluoromethyl, cyano, phenylmethyl-N(methyl)-, tert-butoxycarbonylpiperazinyl, hydroxy, methoxy, ethoxy, propoxy, isopropoxy, butoxy, isobutoxy, difluoromethylmethyl-O—, difluoromethylethyl-O—, trifluoromethoxy, trifluoromethylmethyl-O—, trifluoromethylethyl-O—, ethyldifluoromethyl-O—, vinyldifluoromethyl-O—, propargyl-O—, hydroxymethylpropargyl-O—, methoxyethyl-O—, methoxypropyl-O—, methoxybutyl-O—, ethoxyethyl-O—, methoxyethyl-O-ethyl-O—, aminoethyl-O—, aminopentyl-O—, aminohexyl-O—, aminooctyl-O—, tert-butoxycarbonylaminopentyl-O—, tert-butoxycarbonylaminohexyl-O—, tert-butoxycarbonylaminooctyl-O—, methylcarbonylaminoethyl-O—, methylcarbonylaminopentyl-O—, methyl sulfonyl aminoethyl-O—, methyl sulfonylaminopentyl-O—, methyl sulfonyl ethyl-O—, methylsulfonylpropyl-O—, methylsulfanylpropyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-dimethylpropyl-O—, hydroxy-difluoropropyl-O—, hydroxybutyl-O—, hydroxypentyl-O—, hydroxyhexyl-O—, aminoethyl-O-propyl-O—, ethylamino-ethyl-O-propyl-O—, imidazolylethyl-O—, pyrazolylpropyl-O—, triazolylpropyl-O—, morpholinylethyl-O—, morpholinylpropyl-O—, (2-oxo-pyrrolidinyl)ethyl-O—, (2-oxo-pyrrolidinyl)propyl-O—, phenylmethyl-O—, phenylethyl-O—, pyrrolidinylethyl-O—, pyrrolidinylpropyl-O—, pyrrolidinylcarbonylmethyl-O—, tetrahydropyranylmethyl-O— or carboxypropyl-O—;
R¹ is hydrogen, fluoro, chloro, bromo, methyl or cyano;
R⁸ is hydrogen or methyl; and
R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl.

24. The method of claim 22, wherein:
R⁴ is hydrogen, halogen, C₁₋₆ alkylamino or C₁₋₆ alkoxy;
R³ is hydrogen, halogen, C₁₋₆ alkyl, C₁₋₆ alkoxy, C₃₋₇ cycloalkyl, hydroxy or phenyl-CₓH₂ₓ—O—;
R² is hydrogen; halogen; C₁₋₆ alkyl; cyano; phenyl-CₓH₂ₓ—N(C₁₋₆ alkyl)-; C₁₋₆ alkoxycarbonylpiperazinyl; or wherein R^{a1}—O—, where in R^{a1} is hydrogen; C₁₋₆ alkyl, which is unsubstituted or substituted with one to three substituents independently selected from fluoro, hydroxy and C₂₋₆alkenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; C₃₋₇cycloalkylC₁₋₆alkyl; cyanoC₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; heteroarylC₁₋₆alkyl (e.g., heteroaryl is N-containing monocyclic heteroaryl); or heterocycloalkylC₁₋₆alkyl (e.g., heterocycloalkyl is monocyclic heterocycloalkyl);
R¹ is hydrogen, halogen, C₁₋₆alkyl or cyano;
R⁸ is hydrogen or C₁₋₆alkyl;
R⁷ is hydrogen; C₁₋₆alkyl, which is unsubstituted or once, twice or three times substituted by fluoro; C₃₋₇cycloalkyl; C₁₋₆alkylC₃₋₇cycloalkyl; or phenyl-CₓH₂ₓ—; and
x is 1-6.

25. The method of claim 22, wherein:
R⁴ is hydrogen, fluoro, chloro, bromo, methylamino, methoxy or ethoxy;
R³ is hydrogen, fluoro, chloro, methyl, ethyl, methoxy, ethoxy, propoxy, cyclopropyl, hydroxy or phenylmethyl-O—;
R² is hydrogen, bromo, methyl, propyl, cyano, phenylmethyl-N(methyl)-, tert-butoxycarbonylpiperazinyl, hydroxy, methoxy, ethoxy, propoxy, isopropoxy, butoxy, isobutoxy, difluoromethylmethyl-O—, difluoromethylethyl-O—, trifluoromethylmethyl-O—, ethyldifluoromethyl-O—, vinyldifluoromethyl-O—, propargyl-O—, hydroxymethylpropargyl-O—, methoxyethyl-O—, methoxypropyl-O—, methoxybutyl-O—, ethoxyethyl-O—, methoxyethyl-O-ethyl-O—, aminoethyl-O—, aminopentyl-O—, aminohexyl-O—, aminooctyl-O—, tert-butoxycarbonylaminopentyl-O—, tert-butoxycarbonylaminohexyl-O—, tert-butoxycarbonylaminooctyl-O—, methylcarbonylaminoethyl-O—, methylcarbonylaminopentyl-O—, methylsulfonylaminoethyl-O—, methyl sulfonylaminopentyl-O—, methyl sulfonyl ethyl-O—, methylsulfonylpropyl-O—, methylsulfanylpropyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-dimethylpropyl-O—, hydroxy-difluoropropyl-O—, hydroxybutyl-O—, hydroxypentyl-O—, hydroxyhexyl-O—, aminoethyl-O-propyl-O—, ethylamino-ethyl-O-propyl-O—, imidazolylethyl-O—, pyrazolylpropyl-O—, triazolylpropyl-O—, morpholinylethyl-O—, morpholinylpropyl-O—, (2-oxo-pyrrolidinyl)ethyl-O—, (2-oxo-pyrrolidinyl)propyl-O—, phenylmethyl-O—, phenylethyl-O—, pyrrolidinylethyl-O—, pyrrolidinylpropyl-O—, pyrrolidinylcarbonylmethyl-O—, tetrahydropyranylmethyl-O— or carboxypropyl-O—;
R¹ is hydrogen, chloro, bromo, methyl or cyano;
R⁸ is hydrogen or methyl; and
R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl.

26. The method of claim 22, wherein the compound of Formula (II) has Formula (IIB): 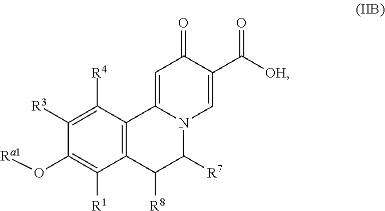
or a pharmaceutically acceptable salt thereof, wherein:
R⁴ is hydrogen, halogen or C₁₋₆alkoxy;
R³ is hydrogen, halogen, C₁₋₆alkyl, C₁₋₆alkoxy, C₃₋₇cycloalkyl, hydroxy or phenyl-CₓH₂ₓ—O—;
R¹ is hydrogen or halogen;
R⁸ is hydrogen or C₁₋₆alkyl;
R⁷ is hydrogen; C₁₋₆alkyl, which is unsubstituted or once, twice or three times substituted by fluoro; C₃₋₇cycloalkyl; C₁₋₆alkylC₃₋₄cycloalkyl; or phenyl-CₓH₂ₓ—;
R^{a1} is hydrogen; C₁₋₆alkyl, which is unsubstituted or substituted with one to three substituents independently selected from fluoro, hydroxy and ethenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; C₃₋₇cycloalkylC₁₋₆alkyl; cyanoC₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; heteroarylC₁₋₆alkyl (e.g., heteroaryl is N-containing monocyclic heteroaryl); or heterocycloalkylC₁₋₆alkyl (e.g., heterocycloalkyl is monocyclic heterocycloalkyl); and
x is 1-6.

27. The method of claim 26, wherein:
R⁴ is hydrogen, fluoro, chloro or methoxy;
R³ is hydrogen, fluoro, chloro, methyl, ethyl, methoxy, ethoxy, propoxy, cyclopropyl, hydroxy or phenylmethyl-O—;
R¹ is hydrogen or chloro;
R⁸ is hydrogen or methyl;
R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl; and
R^{a1} is hydrogen, methyl, ethyl, propyl, isopropyl, butyl, isobutyl, difluoromethylmethyl, difluoromethyl ethyl, trifluoromethylmethyl, ethyldifluoromethyl, vinyl difluoromethyl, propargyl, hydroxymethylpropargyl, methoxyethyl, methoxypropyl, methoxybutyl, ethoxyethyl, methoxyethyl-O-ethyl, aminoethyl, aminopentyl, aminohexyl, aminooctyl, tert-butoxycarbonylaminopentyl, tert-butoxycarbonylaminohexyl, tert-butoxycarbonylaminooctyl, methylcarbonylaminoethyl, methylcarbonylaminopentyl, methyl sulfonylaminoethyl, methyl sulfonylaminopentyl, methyl sulfonyl ethyl, methyl sulfonylpropyl, methyl sulfanylpropyl, cyanopropyl, cyanocyclopropylmethyl, cyclopropylmethyl, cyclohexylethyl, hydroxyethyl, hydroxypropyl, hydroxy-dimethylpropyl, hydroxy-difluoropropyl, hydroxybutyl, hydroxypentyl, hydroxyhexyl, aminoethyl-O-propyl, ethylamino-ethyl-O-propyl-, imidazolylethyl, pyrazolylpropyl, triazolylpropyl, morpholinylethyl, morpholinylpropyl, (2-oxo-pyrrolidinyl)ethyl, (2-oxo-pyrrolidinyl)propyl, phenylmethyl, phenylethyl, pyrrolidinylethyl, pyrrolidinylpropyl, pyrrolidinylcarbonylmethyl, tetrahydropyranylmethyl or carboxypropyl.

28. The method of claim 26, wherein:
R⁴ is hydrogen or halogen;
R³ is C₁₋₆alkyl, halogen or C₃₋₇cycloalkyl;
R¹ is hydrogen;
R⁸ is hydrogen or C₁₋₆alkyl;
R⁷ is C₁₋₆alkyl or C₁₋₆alkylC₃₋₇cycloalkyl; and
R^{a1} is C₁₋₆alkyl, C₁₋₆alkoxyC₁₋₆alkyl or phenylC₁₋₆alkyl.

29. The method of claim 26, wherein:
R⁴ is hydrogen, fluoro or chloro;
R³ is methyl, ethyl, fluoro, chloro or cyclopropyl;
R¹ is hydrogen;
R⁸ is hydrogen or methyl;
R⁷ is methyl, ethyl, isopropyl, isobutyl, tert-butyl or methylcyclopropyl; and
R^{a1} is methyl, ethyl, methoxyethyl, methoxypropyl or phenylmethyl.

30. The method of claim 26, wherein:
R⁴ is hydrogen;
R³ is C₁₋₆alkoxy;
R¹ is hydrogen or halogen;
R⁸ is hydrogen or C₁₋₆alkyl;
R⁷ is hydrogen; C₁₋₆alkyl, which is unsubstituted or once, twice or three times substituted by fluoro; C₃₋₇cycloalkyl; C₁₋₆alkylC₃₋₄cycloalkyl; or phenyl-CₓH₂ₓ—;
R^{a1} is hydrogen; C₁₋₆alkyl, which is unsubstituted or substituted with one to three substituents independently selected from fluoro, hydroxy and C₂₋₆alkenyl; C₁₋₆alkoxyC₁₋₆alkyl; C₁₋₆alkoxyC₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; cyanoC₁₋₆alkyl; cyanoC₃₋₇cycloalkylC₁₋₆alkyl; C₃₋₇cycloalkylC₁₋₆alkyl; phenylC₁₋₆alkyl; pyrrolidinylcarbonylC₁₋₆alkyl; C₂₋₆alkynyl; hydroxyC₁₋₆alkylC₂₋₆alkynyl; aminoC₁₋₆alkoxyC₁₋₆alkyl; C₁₋₆alkylaminoC₁₋₆alkoxyC₁₋₆alkyl; carboxyC₁₋₆alkyl; C₁₋₆alkoxycarbonylaminoC₁₋₈alkyl; imidazolylC₁₋₆alkyl; pyrazolylC₁₋₆alkyl; triazolylC₁₋₆alkyl; morpholinylC₁₋₆alkyl; (2-oxo-pyrrolidinyl)C₁₋₆ alkyl; pyrrolidinylC₁₋₆alkyl; or tetrahydropyranylC₁₋₆alkyl; and
x is 1-6.

31. The method of claim 26, wherein:
R⁴ is hydrogen;
R³ is methoxy, ethoxy or propoxy;
R¹ is hydrogen or chloro;
R⁸ is hydrogen or methyl;
R⁷ is hydrogen, methyl, ethyl, propyl, isopropyl, isobutyl, tert-butyl, trifluoromethyl, trifluoromethylmethyl, cyclopropyl, cyclobutyl, methylcyclopropyl or phenylmethyl; and
R^{a1} is hydrogen, methyl, ethyl, propyl, isopropyl, butyl, isobutyl, difluoromethylmethyl, difluoromethyl ethyl, trifluoromethylmethyl, ethyldifluoromethyl, vinyldifluoromethyl, propargyl, hydroxymethylpropargyl, methoxyethyl, methoxypropyl, methoxybutyl, ethoxyethyl, methoxyethyl-O-ethyl, aminoethyl, aminopentyl, aminohexyl, aminooctyl, tert-butoxycarbonylaminopentyl, tert-butoxycarbonylaminohexyl, tert-butoxycarbonylaminooctyl, methylcarbonylaminoethyl, methylcarbonylaminopentyl, methyl sulfonylaminoethyl, methyl sulfonylaminopentyl, methylsulfonylethyl, methyl sulfonylpropyl, methyl sulfanylpropyl, cyanopropyl, cyanocyclopropylmethyl, cyclopropylmethyl, cyclohexylethyl, hydroxyethyl, hydroxypropyl, hydroxy-dimethylpropyl, hydroxy-difluoropropyl, hydroxybutyl, hydroxypentyl, hydroxyhexyl, aminoethyl-O-propyl, ethylamino-ethyl-O-propyl-, imidazolylethyl, pyrazolylpropyl, triazolylpropyl, morpholinylethyl, morpholinylpropyl, (2-oxo-pyrrolidinyl)ethyl, (2-oxo-pyrrolidinyl)propyl, phenylmethyl, phenyl ethyl, pyrrolidinylethyl, pyrrolidinylpropyl, pyrrolidinylcarbonylmethyl, tetrahydropyranylmethyl or carboxypropyl.

32. The method of claim 26, wherein
R⁴ is hydrogen or halogen;
R³ is halogen, C₁₋₆alkyl, C₁₋₆alkoxy or C₃₋₇cycloalkyl;
R¹ is hydrogen;
R⁸ is hydrogen or C₁₋₆alkyl;
R⁷ is C₁₋₆alkyl, which is unsubstituted or once, twice or three times substituted by fluoro; C₃₋₇cycloalkyl or C₁₋₆alkylC₃₋₇cycloalkyl; and
R^{a1} is C₁₋₆alkyl, which is unsubstituted or substituted with one to three substituents independently selected from fluoro and hydroxy; C₁₋₆alkoxyC₁₋₆alkyl; aminoC₁₋₈alkyl; C₁₋₆alkylcarbonylaminoC₁₋₈alkyl; C₁₋₆alkylsulfonylaminoC₁₋₈alkyl; C₁₋₆alkylsulfanylC₁₋₆alkyl; C₁₋₆alkylsulfonylC₁₋₆alkyl; C₃₋₇cycloalkylC₁₋₆alkyl; phenylC₁₋₆alkyl; C₁₋₆alkylaminoC₁₋₆alkoxyC₁₋₆alkyl; C₁₋₆alkoxycarbonylaminoC₁₋₈alkyl; morpholinylC₁₋₆alkyl or tetrahydropyranylC₁₋₆alkyl.

33. The method of claim 26, wherein:
R⁴ is hydrogen, fluoro, or chloro;
R³ is fluoro, chloro, methyl, ethyl, methoxy, ethoxy or cyclopropyl;
R¹ is hydrogen;
R⁸ is hydrogen or methyl;
R⁷ is methyl, ethyl, isopropyl, isobutyl, tert-butyl, trifluoromethylmethyl, cyclobutyl or methylcyclopropyl; and
R^{a1} is methyl, ethyl, propyl, butyl, isobutyl, cyclopropylmethyl, difluoromethylmethyl, difluoromethylethyl, trifluoromethylmethyl, ethyldifluoromethyl, methoxyethyl, methoxypropyl, ethoxyethyl, aminohexyl, aminooctyl, tert-butoxycarbonylaminopentyl, tert-butoxycarbonylaminooctyl, methylcarbonylaminopentyl, methyl sulfonylaminopentyl, methylsulfonylpropyl, methyl sulfanylpropyl, hydroxypropyl, hydroxy-dimethylpropyl, hydroxy-difluoropropyl, hydroxybutyl, hydroxypentyl, hydroxyhexyl, ethylamino-ethyl-O-propyl-, morpholinylethyl, morpholinylpropyl, phenylmethyl or tetrahydropyranylmethyl.

34. The method of claim 22, wherein:
R⁴ is hydrogen, halogen, C₁₋₆alkylamino or C₁₋₆alkoxy;
R³ is hydrogen, C₁₋₆alkyl or C₁₋₆alkoxy;
R² is hydrogen; halogen; C₁₋₆alkyl; cyano; C₁₋₆alkoxycarbonylpiperazinyl or phenyl-CₓH₂ₓ—N(C₁₋₆alkyl)-, wherein x is 1-8;
R¹ is hydrogen, halogen, C₁₋₆alkyl or cyano;
R⁸ is hydrogen; and
R⁷ is C₁₋₆alkyl.

35. The method of claim 22, wherein:
R⁴ is hydrogen, bromo, methylamino or ethoxy;
R³ is hydrogen, methyl or methoxy;
R² is hydrogen, bromo, methyl, propyl, cyano, tert-butoxycarbonylpiperazinyl or phenylmethyl-N(methyl)-;
R¹ is hydrogen, bromo, methyl or cyano;
R⁸ is hydrogen; and
R⁷ is methyl or ethyl.

36. The method of claim 22, wherein:
R⁴ is hydrogen, halogen, C₁₋₆alkyl, C₁₋₆alkylamino or C₁₋₆alkoxy;
R³ is hydrogen; halogen; C₁₋₆alkyl, which is unsubstituted or once or more times substituted by fluoro; C₁₋₆alkoxy, which is unsubstituted or once or more times substituted by fluoro; cyano; C₃₋₇cycloalkyl; hydroxy or phenyl-CₓH₂ₓ—O—;
R² is hydrogen; halogen; C₁₋₆alkyl, which is unsubstituted or once or more times substituted by fluoro; cyano; morpholinyl; pyrrolidinyl; phenyl-CₓH₂ₓ—N(C₁₋₆alkyl)-; C₁₋₆alkoxycarbonylpiperazinyl; or R^{a1}—O—; wherein
R^{a1} is hydrogen; C₁₋₆alkyl, which is unsubstituted or once or more times substituted by fluoro; or R^{2A}—CₓH₂ₓ—; wherein R^{2A} is Cy³, halo, CN, OR^{a3}, C(O)R^{b3}, C(O)OR^{a3}, NR^{c3}R^{d3}, NR^{c3}S(O)₂R^{b3}, S(O)R^{b3}, or S(O)₂R^{b3};
R⁷ is hydrogen; C₁₋₆alkyl, which is unsubstituted or once or more times substituted by fluoro; C₃₋₇cycloalkyl or C₃₋₄cycloalkyl-CₓH₂ₓ—;
x is 1-6;
R¹ is hydrogen, halogen, C₁₋₆alkyl or cyano; and
R⁸ is hydrogen or C₁₋₆alkyl.

37. The method of claim 36, wherein R^{2A} is C₁₋₆alkoxy, C₁₋₆alkylcarbonylamino, C₁₋₆alkylsulfonylamino, C₁₋₆alkylsulfonyl, aminocarbonyl, cyano, cyanoC₃₋₇cycloalkyl, C₃₋₇cycloalkyl, diC₁₋₆alkylamino, hydroxy, imidazolyl, morpholinyl, 2-oxo-pyrrolidinyl, phenyl, pyrrolidinyl, pyrrolidinylcarbonyl or tetrahydropyranyl.

38. The method of claim 22, wherein:
R⁴ is hydrogen, fluoro, chloro, bromo, methyl, methylamino, methoxy or ethoxy;
R³ is hydrogen, fluoro, chloro, bromo, methyl, ethyl, trifluoromethyl, methoxy, ethoxy, propoxy, trifluoromethoxy, cyano, cyclopropyl, hydroxy or phenylmethyl-O—;
R² is hydrogen, bromo, methyl, propyl, trifluoromethyl, cyano, morpholinyl, pyrrolidinyl, phenylmethyl-N(methyl)-, tert-butoxycarbonylpiperazinyl, hydroxy, methoxy, ethoxy, propoxy, isopropoxy, isobutoxy, butoxy, difluoromethylmethyl-O—, difluoromethylethyl-O—, trifluoromethoxy, trifluoromethylmethyl-O—, trifluoromethylethyl-O—, methoxyethyl-O—, methoxypropyl-O—, ethoxyethyl-O—, methoxyethyl-O-ethyl-O—, methylcarbonylaminoethyl-O—, methylsulfonylaminoethyl-O—, methyl sulfonylethyl-O—, aminocarbonylmethyl-O—, cyanomethyl-O—, cyanopropyl-O—, cyanocyclopropylmethyl-O—, cyclopropylmethyl-O—, cyclohexylethyl-O—, di ethylaminoethyl-O—, hydroxyethyl-O—, hydroxypropyl-O—, hydroxy-2,2-dimethylpropyl-O—, imidazolylethyl-O—, morpholinylethyl-O—, 2-oxo-pyrrolidin-1-ylethyl-O—, phenylmethyl-O—, phenylethyl-O—, pyrrolidinylethyl-O—, pyrrolidinylcarbonylmethyl-O— or tetrahydropyran-4-ylmethyl-O—;
R¹ is hydrogen, fluoro, chloro, bromo, methyl or cyano;
R⁸ is hydrogen or methyl; and
R⁷ is hydrogen, methyl, ethyl, propyl, isopropyl, isobutyl, trifluoromethyl, trifluoroethyl, cyclopropyl, cyclobutyl or cyclopropylmethyl.

39. The method of claim 22, wherein:
R⁴ is hydrogen, halogen, C₁₋₆alkylamino or C₁₋₆alkoxy;
R³ is hydrogen, halogen, C₁₋₆alkyl, C₁₋₆alkoxy, C₃₋₇cycloalkyl, hydroxy or phenyl-CₓH₂ₓ—O—;
R² is hydrogen; halogen; C₁₋₆alkyl; cyano; phenyl-CₓH₂ₓ—N(C₁₋₆alkyl)-; C₁₋₆alkoxycarbonylpiperazinyl; or R^{a1}—O—; wherein R^{a1} is hydrogen; C₁₋₆alkyl, which is unsubstituted or once or more times substituted by fluoro; or R^{2A}-CₓH₂ₓ—; wherein R^{2A} is C₁₋₆alkoxy, C₁₋₆alkylcarbonylamino, C₁₋₆alkylsulfonylamino, C₁₋₆alkylsulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl;
R¹ is hydrogen, halogen, C₁₋₆alkyl or cyano;
R⁸ is hydrogen or C₁₋₆alkyl;
R⁷ is hydrogen; C₁₋₆alkyl, which is unsubstituted or once or more times substituted by fluoro; or C₃₋₇cycloalkyl; and
x is 1-6.

40. The method of claim 22, wherein:
R⁴ is hydrogen, chloro, bromo, methylamino, methoxy or ethoxy;
R³ is hydrogen, fluoro, chloro, methyl, ethyl, methoxy, ethoxy, propoxy, cyclopropyl, hydroxy or phenylmethyl-O—;
R² is hydrogen, bromo, methyl, propyl, cyano, phenylmethyl-N(methyl)-, tert-butoxycarbonylpiperazinyl, hydroxy, methoxy, ethoxy, propoxy, isopropoxy, isobutoxy, butoxy, difluoromethylmethyl-O—, trifluoromethylmethyl-O—, methoxyethyl-O—, methoxypropyl-O—, ethoxyethyl-O—, methoxyethyl-O-ethyl-O—, methylcarbonylaminoethyl-O—, methylsulfonylaminoethyl-O—, methyl sulfonylethyl-O—, cyanomethyl-O—, cyanopropyl-O—, cyanocyclopropylmethyl-O—, cyclopropylmethyl-O—, cyclohexylethyl-O—, hydroxyethyl-O—, hydroxypropyl-O—, hydroxy-2,2-dimethylpropyl-O—, imidazolylethyl-O—, morpholinylethyl-O—, 2-oxo-pyrrolidin-1-ylethyl-O—, phenylmethyl-O—, phenylethyl-O—, pyrrolidinylethyl-O—, pyrrolidinylcarbonylmethyl-O— or tetrahydropyran-4-ylmethyl-O—;
R¹ is hydrogen, chloro, bromo, methyl or cyano;
R⁸ is hydrogen or methyl; and
R⁷ is hydrogen, methyl, ethyl, propyl, isopropyl, isobutyl, trifluoromethyl or cyclopropyl.

41. The method of claim 26, wherein:
R⁴ is hydrogen, halogen or C₁₋₆alkoxy;
R³ is hydrogen, halogen, C₁₋₆alkyl, C₁₋₆alkoxy, C₃₋₇cycloalkyl, hydroxy or phenyl-CₓH₂ₓ—O—;
R¹ is hydrogen or halogen;
R⁸ is hydrogen;
R⁷ is C₁₋₆alkyl, which is unsubstituted or once or more times substituted by fluoro; or C₃₋₇cycloalkyl;
R^{a1} is hydrogen; C₁₋₆alkyl, which is unsubstituted or once or more times substituted by fluoro; R^{2A}—CₓH₂ₓ—; wherein R^{2A} is C₁₋₆alkoxy, C₁₋₆alkoxy-CₓH₂ₓ—O—, C₁₋₆alkylcarbonylamino, C₁₋₆alkylsulfonylamino, C₁₋₆alkylsulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl; and
x is 1-6.

42. The method of claim 26, wherein:
R⁴ is hydrogen, chloro or methoxy;
R³ is hydrogen, fluoro, chloro, methyl, ethyl, methoxy, ethoxy, propoxy, cyclopropyl, hydroxy or phenylmethyl-O—;
R¹ is hydrogen or chloro;
R⁸ is hydrogen;
R⁷ is methyl, ethyl, propyl, isopropyl, isobutyl, trifluoromethyl or cyclopropyl; and
R^{a1} is hydrogen, methyl, ethyl, propyl, isopropyl, isobutyl, butyl, difluoroethyl, trifluoroethyl, methoxyethyl, methoxypropyl, ethoxyethyl, methoxyethyl-O-ethyl, methylcarbonylaminoethyl, methyl sulfonylaminoethyl, methylsulfonylethyl, cyanomethyl, cyanopropyl, cyanocyclopropylmethyl, cyclopropylmethyl, cyclohexylethyl, hydroxyethyl, hydroxypropyl, hydroxy-2,2-dimethylpropyl, imidazolylethyl, morpholinylethyl, 2-oxo-pyrrolidin-1-ylethyl, phenylmethyl, phenyl ethyl, pyrrolidinylethyl, pyrrolidinylcarbonylmethyl or tetrahydropyran-4-ylmethyl.

43. The method of claim 26, wherein:
R⁴ is hydrogen;
R³ is halogen;
R¹ is hydrogen;
R⁸ is hydrogen;
R⁷ is C₁₋₆alkyl;
R^{a1} is C₁₋₆alkyl or C₁₋₆alkoxy-CₓH₂ₓ—; and
x is 1-6.

44. The method of claim 26, wherein:
R⁴ is hydrogen;
R³ is C₁₋₆alkyl or C₃₋₇cycloalkyl;
R¹ is hydrogen;
R⁸ is hydrogen;
R⁷ is C₁₋₆alkyl;
R^{a1} is C₁₋₆alkyl or phenyl-CₓH₂ₓ—; and
x is 1-6.

45. The method of claim 26, wherein:
R⁴ is hydrogen;
R³ is C₁₋₆alkoxy;
R¹ is hydrogen or halogen;
R⁸ is hydrogen;
R⁷ is C₁₋₆alkyl, which is unsubstituted or once or more times substituted by fluoro; or C₃₋₇cycloalkyl;
R^{a1} is hydrogen; C₁₋₆alkyl, which is unsubstituted or once or more times substituted by fluoro; or R^{2A}CₓH₂ₓ—;
R^{2A} is C₁₋₆alkoxy, C₁₋₆alkylcarbonylamino, C₁₋₆alkylsulfonylamino, C₁₋₆alkylsulfonyl, cyano, cyanoC₃₋₇cycloalkyl, C₃₋₇cycloalkyl, hydroxy, imidazolyl, morpholinyl, 2-oxo-pyrrolidin-1-yl, phenyl, pyrrolidinyl, pyrrolidinylcarbonyl or tetrahydropyran-4-yl; and
x is 1-6.

46. The method of claim 26, wherein:
R⁴ is hydrogen;
R³ is methoxy, ethoxy or propoxy;
R¹ is hydrogen or chloro;
R⁸ is hydrogen;
R⁷ is methyl, ethyl, propyl, isopropyl, isobutyl, trifluoromethyl or cyclopropyl; and
R^{a1} is hydrogen, methyl, ethyl, propyl, isopropyl, isobutyl, butyl, difluoroethyl, trifluoroethyl, methoxyethyl, methoxypropyl, ethoxyethyl, methoxyethyl-O-ethyl, methylcarbonylaminoethyl, methyl sulfonylaminoethyl, methylsulfonylethyl, cyanomethyl, cyanopropyl, cyanocyclopropylmethyl, cyclopropylmethyl, cyclohexylethyl, hydroxyethyl, hydroxypropyl, hydroxy-2,2-dimethylpropyl, imidazolylethyl, morpholinylethyl, 2-oxo-pyrrolidin-1-ylethyl, phenylmethyl, phenyl ethyl, pyrrolidinylethyl, pyrrolidinylcarbonylmethyl or tetrahydropyran-4-ylmethyl.

47. The method of claim 22, wherein:
R⁴ is hydrogen;
R³ is C₁₋₆alkoxy;
R² is C₁₋₆alkoxy;
R¹ is hydrogen;
R⁸ is hydrogen or C₁₋₆alkyl; and
R⁷ is hydrogen.

48. The method of claim 22, wherein:
R⁴ is hydrogen, halogen, C₁₋₆alkylamino or C₁₋₆alkoxy;
R³ is hydrogen, C₁₋₆alkyl or C₁₋₆alkoxy;
R² is hydrogen, bromo, C₁₋₆alkyl, C₁₋₆alkoxycarbonylpiperazinyl, cyano or phenyl-CₓH₂ₓ—N(C₁₋₆alkyl)-; and
R¹ is hydrogen, halogen, C₁₋₆alkyl or cyano;
R⁸ is hydrogen;
R⁷ is C₁₋₆alkyl; and
x is 1-6.

49. The method of claim 22, wherein:
R⁴ is hydrogen, bromo, methylamino or ethoxy;
R³ is hydrogen, methyl or methoxy;
R² is hydrogen, bromo, methyl, propyl, tert-butoxycarbonylpiperazinyl, cyano or phenylmethyl-N(methyl)-;
R¹ is hydrogen, bromo, methyl or cyano;
R⁸ is hydrogen; and
R⁷ is methyl or ethyl.

50. The method of claim 22, wherein the compound of Formula (II) is selected from:
9-Benzyloxy-10-methoxy-6-methyl-2-oxo-6,7-dihydrobenzo[a]quinolizine-3-carboxylic acid;
9-Hydroxy-10-methoxy-6-methyl-2-oxo-6,7-dihydrobenzo[a]quinolizine-3-carboxylic acid;
9,11-Dimethoxy-6-methyl-2-oxo-6,7-dihydrobenzo[a]quinolizine-3-carboxylic acid;
9-Ethoxy-10-methoxy-6-methyl-2-oxo-6,7-dihydrobenzo[a]quinolizine-3-carboxylic acid;
9,10-Diethoxy-6-ethyl-2-oxo-6,7-dihydrobenzo[a]quinolizine-3-carboxylic acid;
(+)-9,10-Diethoxy-6-ethyl-2-oxo-6,7-dihydrobenzo[a]quinolizine-3-carboxylic acid;
(−)-9,10-Diethoxy-6-ethyl-2-oxo-6,7-dihydrobenzo[a]quinolizine-3-carboxylic acid;
9-Benzyloxy-6-ethyl-10-methoxy-2-oxo-6,7-dihydrobenzo[a]quinolizine-3-carboxylic acid;
(+)-9-Benzyloxy-6-ethyl-10-methoxy-2-oxo-6,7-dihydrobenzo[a]quinolizine-3-carboxylic acid;
(−)-9-Benzyloxy-6-ethyl-10-methoxy-2-oxo-6,7-dihydrobenzo[a]quinolizine-3-carboxylic acid;
(+)-6-Ethyl-10-methoxy-2-oxo-9-(2,2,2-trifluoroethoxy)-6,7-dihydrobenzo[a]quinolizine-3-carboxylic acid;
(−)-6-Ethyl-10-methoxy-2-oxo-9-(2,2,2-trifluoroethoxy)-6,7-dihydrobenzo[a]quinolizine-3-carboxylic acid;
6-Ethyl-9-isopropoxy-10-methoxy-2-oxo-6,7-dihydrobenzo[a]quinolizine-3-carboxylic acid;
6-Ethyl-10-methoxy-2-oxo-9-(2-phenylethoxy)-6,7-dihydrobenzo[a]quinolizine-3-carboxylic acid;
9-Butoxy-6-ethyl-10-methoxy-2-oxo-6,7-dihydrobenzo[a]quinolizine-3-carboxylic acid;
9-(2-Cyclohexylethoxy)-6-ethyl-10-methoxy-2-oxo-6,7-dihydrobenzo[a]quinolizine-3-carboxylic acid;
6-Ethyl-10-methoxy-2-oxo-9-prop-2-ynoxy-6,7-dihydrobenzo[a]quinolizine-3-carboxylic acid;
6-Ethyl-10-methoxy-2-oxo-9-(2-oxo-2-pyrrolidin-1-yl-ethoxy)-6,7-dihydrobenzo[a]quinolizine-3-carboxylic acid;
6-Ethyl-10-methoxy-9-[2-(2-methoxyethoxyl)ethoxy]-2-oxo-6,7-dihydrobenzo[a]quinolizine-3-carboxylic acid;
6-Ethyl-10-methoxy-9-(2-morpholinoethoxy)-2-oxo-6,7-dihydrobenzo[a]quinolizine-3-carboxylic acid;
6-Ethyl-9-(2-hydroxyethoxy)-10-methoxy-2-oxo-6,7-dihydrobenzo[a]quinolizine-3-carboxylic acid;
6-Ethyl-9-(3-hydroxy-2,2-dimethyl-propoxy)-10-methoxy-2-oxo-6,7-dihydrobenzo[a]quinolizine-3-carboxylic acid;
(+)-6-Ethyl-9-(3-hydroxy-2,2-dimethyl-propoxy)-10-methoxy-2-oxo-6,7-dihydrobenzo[a]quinolizine-3-carboxylic acid;
(−)-6-Ethyl-9-(3-hydroxy-2,2-dimethyl-propoxy)-10-methoxy-2-oxo-6,7-dihydrobenzo[a]quinolizine-3-carboxylic acid;
6-Ethyl-9-(3-hydroxypropoxy)-10-methoxy-2-oxo-6,7-dihydrobenzo[a]quinolizine-3-carboxylic acid;
6-Ethyl-9-(2-imidazol-1-ylethoxy)-10-methoxy-2-oxo-6,7-dihydrobenzo[a]quinolizine-3-carboxylic acid;
6-Ethyl-10-methoxy-9-(2-methoxyethoxy)-2-oxo-6,7-dihydrobenzo[a]quinolizine-3-carboxylic acid;
(+)-6-Ethyl-10-methoxy-9-(2-methoxyethoxy)-2-oxo-6,7-dihydrobenzo[a]quinolizine-3-carboxylic acid;
(−)-6-Ethyl-10-methoxy-9-(2-methoxyethoxy)-2-oxo-6,7-dihydrobenzo[a]quinolizine-3-carboxylic acid;
9-(Cyclopropylmethoxy)-6-ethyl-10-methoxy-2-oxo-6,7-dihydrobenzo[a]quinolizine-3-carboxylic acid;
(+)-9-(Cyclopropylmethoxy)-6-ethyl-10-methoxy-2-oxo-6,7-dihydrobenzo[a]quinolizine-3-carboxylic acid;
(−)-9-(Cyclopropylmethoxy)-6-ethyl-10-methoxy-2-oxo-6,7-dihydrobenzo[a]quinolizine-3-carboxylic acid;
6-Ethyl-9-isobutoxy-10-methoxy-2-oxo-6,7-dihydrobenzo[a]quinolizine-3-carboxylic acid;
(+)-6-Ethyl-9-isobutoxy-10-methoxy-2-oxo-6,7-dihydrobenzo[a]quinolizine-3-carboxylic acid;
(−)-6-Ethyl-9-isobutoxy-10-methoxy-2-oxo-6,7-dihydrobenzo[a]quinolizine-3-carboxylic acid;
9-Ethoxy-6-ethyl-10-methoxy-2-oxo-6,7-dihydrobenzo[a]quinolizine-3-carboxylic acid;
(+)-9-Ethoxy-6-ethyl-10-methoxy-2-oxo-6,7-dihydrobenzo[a]quinolizine-3-carboxylic acid;
(−)-9-Ethoxy-6-ethyl-10-methoxy-2-oxo-6,7-dihydrobenzo[a]quinolizine-3-carboxylic acid;
6-Ethyl-10-methoxy-9-(3-methoxypropoxy)-2-oxo-6,7-dihydrobenzo[a]quinolizine-3-carboxylic acid;
(+)-6-Ethyl-10-methoxy-9-(3-methoxypropoxy)-2-oxo-6,7-dihydrobenzo[a]quinolizine-3-carboxylic acid;
(−)-6-Ethyl-10-methoxy-9-(3-methoxypropoxy)-2-oxo-6,7-dihydrobenzo[a]quinolizine-3-carboxylic acid;
9-(2-Ethoxyethoxy)-6-ethyl-10-methoxy-2-oxo-6,7-dihydrobenzo[a]quinolizine-3-carboxylic acid;
(+)-9-(2-Ethoxyethoxy)-6-ethyl-10-methoxy-2-oxo-6,7-dihydrobenzo[a]quinolizine-3-carboxylic acid;
(−)-9-(2-Ethoxyethoxy)-6-ethyl-10-methoxy-2-oxo-6,7-dihydrobenzo[a]quinolizine-3-carboxylic acid;
9-(2,2-Difluoroethoxy)-6-ethyl-10-methoxy-2-oxo-6,7-dihydrobenzo[a]quinolizine-3-carboxylic acid;
(+)-9-(2,2-Difluoroethoxy)-6-ethyl-10-methoxy-2-oxo-6,7-dihydrobenzo[a]quinolizine-3-carboxylic acid;
(−)-9-(2,2-Difluoroethoxy)-6-ethyl-10-methoxy-2-oxo-6,7-dihydrobenzo[a]quinolizine-3-carboxylic acid;
6-Ethyl-10-methoxy-2-oxo-9-(tetrahydropyran-4-ylmethoxy)-6,7-dihydrobenzo[a]quinolizine-3-carboxylic acid;
(+)-6-Ethyl-10-methoxy-2-oxo-9-(tetrahydropyran-4-ylmethoxy)-6,7-dihydrobenzo[a]quinolizine-3-carboxylic acid;
(−)-6-Ethyl-10-methoxy-2-oxo-9-(tetrahydropyran-4-ylmethoxy)-6,7-dihydrobenzo[a]quinolizine-3-carboxylic acid;
6-Ethyl-10-methoxy-2-oxo-9-(2-pyrrolidin-1-ylethoxy)-6,7-dihydrobenzo[a]quinolizine-3-carboxylic acid;
9-(3-Cyanopropoxy)-6-ethyl-10-methoxy-2-oxo-6,7-dihydrobenzo[a]quinolizine-3-carboxylic acid;
6-Ethyl-10-methoxy-9-(2-methylsulfonylethoxy)-2-oxo-6,7-dihydrobenzo[a]quinolizine-3-carboxylic acid;
6-Ethyl-10-methoxy-2-oxo-9-[2-(2-oxopyrrolidin-1-yl)ethoxy]-6,7-dihydrobenzo[a]quinolizine-3-carboxylic acid;
(+)-6-Ethyl-10-methoxy-2-oxo-9-[2-(2-oxopyrrolidin-1-yl)ethoxy]-6,7-dihydrobenzo[a]quinolizine-3-carboxylic acid;
6-Ethyl-9-[2-(methanesulfonamido)ethoxy]-10-methoxy-2-oxo-6,7-dihydrobenzo[a]quinolizine-3-carboxylic acid;
9-[(1-Cyanocyclopropyl)methoxy]-6-ethyl-10-methoxy-2-oxo-6,7-dihydrobenzo[a]quinolizine-3-carboxylic acid;
9-(2-Acetamidoethoxy)-6-ethyl-10-methoxy-2-oxo-6,7-dihydrobenzo[a]quinolizine-3-carboxylic acid;
10-Chloro-9-methoxy-6-methyl-2-oxo-6,7-dihydrobenzo[a]quinolizine-3-carboxylic acid;
9,10-Dimethoxy-6-methyl-2-oxo-6,7-dihydrobenzo[a]quinolizine-3-carboxylic acid;
(+)-9,10-Dimethoxy-6-methyl-2-oxo-6,7-dihydrobenzo[a]quinolizine-3-carboxylic acid;
(−)-9,10-Dimethoxy-6-methyl-2-oxo-6,7-dihydrobenzo[a]quinolizine-3-carboxylic acid;
9,10-Dimethoxy-7-methyl-2-oxo-6,7-dihydrobenzo[a]quinolizine-3-carboxylic acid;
6-Ethyl-9,10-dimethoxy-2-oxo-6,7-dihydrobenzo[a]quinolizine-3-carboxylic acid;
(6R)-(+)-6-Ethyl-9,10-dimethoxy-2-oxo-6,7-dihydrobenzo[a]quinolizine-3-carboxylic acid;
(6S)-(−)-6-Ethyl-9,10-dimethoxy-2-oxo-6,7-dihydrobenzo[a]quinolizine-3-carboxylic acid;
9-Methoxy-6,10-dimethyl-2-oxo-6,7-dihydrobenzo[a]quinolizine-3-carboxylic acid;
9,10-Diethoxy-6-methyl-2-oxo-6,7-dihydrobenzo[a]quinolizine-3-carboxylic acid;
9-Ethoxy-6-methyl-10-hydroxy-2-oxo-6,7-dihydrobenzo[a]quinolizine-3-carboxylic acid;
9,10-Diethoxy-2-oxo-6,7-dihydrobenzo[a]quinolizine-3-carboxylic acid;
2-Oxo-9,10-dipropoxy-6,7-dihydrobenzo[a]quinolizine-3-carboxylic acid;
6-Ethyl-10-methoxy-2-oxo-9-propoxy-6,7-dihydrobenzo[a]quinolizine-3-carboxylic acid;
(+)-6-Ethyl-10-methoxy-2-oxo-9-propoxyl-6,7-dihydrobenzo[a]quinolizine-3-carboxylic acid;
(−)-6-Ethyl-10-methoxy-2-oxo-9-propoxyl-6,7-dihydrobenzo[a]quinolizine-3-carboxylic acid;
8-Chloro-9-methoxy-6-methyl-2-oxo-6,7-dihydrobenzo[a]quinolizine-3-carboxylic acid;
8-Chloro-9,10-dimethoxy-6-methyl-2-oxo-6,7-dihydrobenzo[a]quinolizine-3-carboxylic acid;
10-Benzyloxy-9-methoxy-6-methyl-2-oxo-6,7-dihydrobenzo[a]quinolizine-3-carboxylic acid;
10-Ethoxy-9-methoxy-6-methyl-2-oxo-6,7-dihydrobenzo[a]quinolizine-3-carboxylic acid;
9-Methoxy-6-methyl-2-oxo-10-propoxy-6,7-dihydrobenzo[a]quinolizine-3-carboxylic acid;
6,10-Diethyl-9-methoxy-2-oxo-6,7-dihydrobenzo[a]quinolizine-3-carboxylic acid;
10-Cyclopropyl-6-ethyl-9-methoxy-2-oxo-6,7-dihydrobenzo[a]quinolizine-3-carboxylic acid;
(+)-10-Cyclopropyl-6-ethyl-9-methoxy-2-oxo-6,7-dihydrobenzo[a]quinolizine-3-carboxylic acid;
(−)-10-Cyclopropyl-6-ethyl-9-methoxy-2-oxo-6,7-dihydrobenzo[a]quinolizine-3-carboxylic acid;
9,10-Dimethoxy-2-oxo-6-propyl-6,7-dihydrobenzo[a]quinolizine-3-carboxylic acid;
6-Cyclopropyl-9,10-dimethoxy-2-oxo-6,7-dihydrobenzo[a]quinolizine-3-carboxylic acid;
6-isopropyl-9,10-dimethoxy-2-oxo-6,7-dihydrobenzo[a]quinolizine-3-carboxylic acid;
(+)-6-isopropyl-9,10-dimethoxy-2-oxo-6,7-dihydrobenzo[a]quinolizine-3-carboxylic acid;
(−)-6-isopropyl-9,10-dimethoxy-2-oxo-6,7-dihydrobenzo[a]quinolizine-3-carboxylic acid;
6-isobutyl-9,10-dimethoxy-2-oxo-6,7-dihydrobenzo[a]quinolizine-3-carboxylic acid;
(+)-6-Isobutyl-9,10-dimethoxy-2-oxo-6,7-dihydrobenzo[a]quinolizine-3-carboxylic acid;
(−)-6-Isobutyl-9,10-dimethoxy-2-oxo-6,7-dihydrobenzo[a]quinolizine-3-carboxylic acid;
10-Chloro-6-isobutyl-9-(2-methoxyethoxy)-2-oxo-6,7-dihydrobenzo[a]quinolizine-3-carboxylic acid;
(+)-10-Chloro-6-isobutyl-9-(2-methoxyethoxy)-2-oxo-6,7-dihydrobenzo[a]quinolizine-3-carboxylic acid;
(−)-10-Chloro-6-isobutyl-9-(2-methoxyethoxy)-2-oxo-6,7-dihydrobenzo[a]quinolizine-3-carboxylic acid;
10-Chloro-6-isopropyl-9-(2-methoxyethoxy)-2-oxo-6,7-dihydrobenzo[a]quinolizine-3-carboxylic acid;
(+)-10-Chloro-6-isopropyl-9-(2-methoxyethoxy)-2-oxo-6,7-dihydrobenzo[a]quinolizine-3-carboxylic acid;
(−)-10-Chloro-6-isopropyl-9-(2-methoxyethoxy)-2-oxo-6,7-dihydrobenzo[a]quinolizine-3-carboxylic acid;
10-Fluoro-6-isopropyl-9-(2-methoxyethoxy)-2-oxo-6,7-dihydrobenzo[a]quinolizine-3-carboxylic acid;
11-Chloro-6-isopropyl-9-(2-methoxyethoxy)-2-oxo-6,7-dihydrobenzo[a]quinolizine-3-carboxylic acid;
9,10-Dimethoxy-2-oxo-6-(trifluoromethyl)-6,7-dihydrobenzo[a]quinolizine-3-carboxylic acid;
9-Benzyloxy-6-ethyl-10-methyl-2-oxo-6,7-dihydrobenzo[a]quinolizine-3-carboxylic acid;
(+)-9-Benzyloxy-6-ethyl-10-methyl-2-oxo-6,7-dihydrobenzo[a]quinolizine-3-carboxylic acid;
(−)-9-Benzyloxy-6-ethyl-10-methyl-2-oxo-6,7-dihydrobenzo[a]quinolizine-3-carboxylic acid;
(+)-10-Chloro-9-ethoxy-6-ethyl-2-oxo-6,7-dihydrobenzo[a]quinolizine-3-carboxylic acid;
(−)-10-Chloro-9-ethoxy-6-ethyl-2-oxo-6,7-dihydrobenzo[a]quinolizine-3-carboxylic acid;
10-Chloro-6-isopropyl-9-(3-methoxypropoxy)-2-oxo-6,7-dihydrobenzo[a]quinolizine-3-carboxylic acid;
(+)-10-Chloro-6-isopropyl-9-(3-methoxypropoxy)-2-oxo-6,7-dihydrobenzo[a]quinolizine-3-carboxylic acid;
(−)-10-Chloro-6-isopropyl-9-(3-methoxypropoxy)-2-oxo-6,7-dihydrobenzo[a]quinolizine-3-carboxylic acid;
10-Methoxy-6-isopropyl-9-(3-methoxypropoxy)-2-oxo-6,7-dihydrobenzo[a]quinolizine-3-carboxylic acid;
(+)-10-Methoxy-6-isopropyl-9-(3-methoxypropoxy)-2-oxo-6,7-dihydrobenzo[a]quinolizine-3-carboxylic acid;
(−)-10-Methoxy-6-isopropyl-9-(3-methoxypropoxy)-2-oxo-6,7-dihydrobenzo[a]quinolizine-3-carboxylic acid;
6-Isopropyl-10-methoxy-2-oxo-9-(2,2,2-trifluoroethoxy)-6,7-dihydrobenzo[a]quinolizine-3-carboxylic acid;
(+)-6-Isopropyl-10-methoxy-2-oxo-9-(2,2,2-trifluoroethoxy)-6,7-dihydrobenzo[a]quinolizine-3-carboxylic acid;
(−)-6-Isopropyl-10-methoxy-2-oxo-9-(2,2,2-trifluoroethoxy)-6,7-dihydrobenzo[a]quinolizine-3-carboxylic acid;
(+)-6-Isopropyl-10-methoxy-9-(2-methoxyethoxy)-2-oxo-6,7-dihydrobenzo[a]quinolizine-3-carboxylic acid;
(−)-6-Isopropyl-10-methoxy-9-(2-methoxyethoxy)-2-oxo-6,7-dihydrobenzo[a]quinolizine-3-carboxylic acid;
6-tert-Butyl-10-methoxy-9-(3-methoxypropoxy)-2-oxo-6,7-dihydrobenzo[a]quinolizine-3-carboxylic acid;
(+)-6-tert-Butyl-10-methoxy-9-(3-methoxypropoxy)-2-oxo-6,7-dihydrobenzo[a]quinolizine-3-carboxylic acid;
(−)-6-tert-Butyl-10-methoxy-9-(3-methoxypropoxy)-2-oxo-6,7-dihydrobenzo[a]quinolizine-3-carboxylic acid;
10-Methoxy-9-(2-methoxyethoxy)-6-(1-methylcyclopropyl)-2-oxo-6,7-dihydrobenzo[a]quinolizine-3-carboxylic acid;
(+)-10-Methoxy-9-(2-methoxyethoxy)-6-(1-methylcyclopropyl)-2-oxo-6,7-dihydrobenzo[a]quinolizine-3-carboxylic acid;
(−)-10-Methoxy-9-(2-methoxyethoxy)-6-(1-methylcyclopropyl)-2-oxo-6,7-dihydrobenzo[a]quinolizine-3-carboxylic acid;
11-Chloro-10-fluoro-6-isopropyl-9-(3-methoxypropoxy)-2-oxo-6,7-dihydrobenzo[a]quinolizine-3-carboxylic acid;
(+)-11-Chloro-10-fluoro-6-isopropyl-9-(3-methoxypropoxy)-2-oxo-6,7-dihydrobenzo[a]quinolizine-3-carboxylic acid;
(−)-11-Chloro-10-fluoro-6-isopropyl-9-(3-methoxypropoxy)-2-oxo-6,7-dihydrobenzo[a]quinolizine-3-carboxylic acid;
10-Fluoro-6-isopropyl-9-(3-methoxypropoxy)-2-oxo-6,7-dihydrobenzo[a]quinolizine-3-carboxylic acid;
6-tert-Butyl-9-(2,2-difluoro-3-hydroxy-propoxy)-10-methoxy-2-oxo-6,7-dihydrobenzo[a]quinolizine-3-carboxylic acid;
(+)-9-(2,2-Difluoroethoxy)-6-isopropyl-10-methoxy-2-oxo-6,7-dihydrobenzo[a]quinolizine-3-carboxylic acid;
(−)-9-(2,2-Difluoroethoxy)-6-isopropyl-10-methoxy-2-oxo-6,7-dihydrobenzo[a]quinolizine-3-carboxylic acid;
9-(3-Hydroxy-2,2-dimethyl-propoxy)-6-isopropyl-10-methoxy-2-oxo-6,7-dihydrobenzo[a]quinolizine-3-carboxylic acid;
(+)-9-(3-Hydroxy-2,2-dimethyl-propoxy)-6-isopropyl-10-methoxy-2-oxo-6,7-dihydrobenzo[a]quinolizine-3-carboxylic acid;
(−)-9-(3-Hydroxy-2,2-dimethyl-propoxy)-6-isopropyl-10-methoxy-2-oxo-6,7-dihydrobenzo[a]quinolizine-3-carboxylic acid;
9-(3-Hydroxypropoxy)-6-isopropyl-10-methoxy-2-oxo-6,7-dihydrobenzo[a]quinolizine-3-carboxylic acid;
6-Isopropyl-10-methoxy-9-(4-methoxybutoxy)-2-oxo-6,7-dihydrobenzo[a]quinolizine-3-carboxylic acid;
6-tert-Butyl-9-(3-hydroxy-2,2-dimethyl-propoxy)-10-methoxy-2-oxo-6,7-dihydrobenzo[a]quinolizine-3-carboxylic acid;
(+)-6-tert-Butyl-9-(3-hydroxy-2,2-dimethyl-propoxy)-10-methoxy-2-oxo-6,7-dihydrobenzo[a]quinolizine-3-carboxylic acid;
(−)-6-tert-Butyl-9-(3-hydroxy-2,2-dimethyl-propoxy)-10-methoxy-2-oxo-6,7-dihydrobenzo[a]quinolizine-3-carboxylic acid;
6-tert-Butyl-9-(5-hydroxypentoxy)-10-methoxy-2-oxo-6,7-dihydrobenzo[a]quinolizine-3-carboxylic acid;
(+)-6-tert-Butyl-9-(5-hydroxypentoxy)-10-methoxy-2-oxo-6,7-dihydrobenzo[a]quinolizine-3-carboxylic acid;
(−)-6-tert-Butyl-9-(5-hydroxypentoxy)-10-methoxy-2-oxo-6,7-dihydrobenzo[a]quinolizine-3-carboxylic acid;
6-tert-Butyl-9-(6-hydroxyhexoxy)-10-methoxy-2-oxo-6,7-dihydrobenzo[a]quinolizine-3-carboxylic acid;
(+)-6-tert-Butyl-9-(6-hydroxyhexoxy)-10-methoxy-2-oxo-6,7-dihydrobenzo[a]quinolizine-3-carboxylic acid;
(−)-6-tert-Butyl-9-(6-hydroxyhexoxy)-10-methoxy-2-oxo-6,7-dihydrobenzo[a]quinolizine-3-carboxylic acid;
6-tert-Butyl-9-(4-hydroxybutoxy)-10-methoxy-2-oxo-6,7-dihydrobenzo[a]quinolizine-3-carboxylic acid;
6-tert-Butyl-9-(4-hydroxybut-2-ynoxy)-10-methoxy-2-oxo-6,7-dihydrobenzo[a]quinolizine-3-carboxylic acid;
9-(6-Aminohexoxy)-6-tert-butyl-10-methoxy-2-oxo-6,7-dihydrobenzo[a]quinolizine-3-carboxylic acid;
9-[6-(tert-Butoxycarbonylamino) hexoxy]-6-tert-butyl-10-methoxy-2-oxo-6,7-dihydrobenzo[a]quinolizine-3-carboxylic acid;
(+)-9-[6-(tert-Butoxycarbonylamino)hexoxy]-6-tert-butyl-10-methoxy-2-oxo-6,7-dihydrobenzo[a]quinolizine-3-carboxylic acid;
(−)-9-[6-(tert-Butoxycarbonylamino)hexoxy]-6-tert-butyl-10-methoxy-2-oxo-6,7-dihydrobenzo[a]quinolizine-3-carboxylic acid;
(+)-9-(6-Aminohexoxy)-6-tert-butyl-10-methoxy-2-oxo-6,7-dihydrobenzo[a]quinolizine-3-carboxylic acid hydrochloride;
(−)-9-(6-Aminohexoxy)-6-tert-butyl-10-methoxy-2-oxo-6,7-dihydrobenzo[a]quinolizine-3-carboxylic acid hydrochloride;
9-(8-Aminooctoxy)-6-tert-butyl-10-methoxy-2-oxo-6,7-dihydrobenzo[a]quinolizine-3-carboxylic acid;
9-[8-(tert-Butoxycarbonylamino) octoxy]-6-tert-butyl-10-methoxy-2-oxo-6,7-dihydrobenzo[a]quinolizine-3-carboxylic acid;
(+)-9-[8-(tert-Butoxycarbonylamino)octoxy]-6-tert-butyl-10-methoxy-2-oxo-6,7-dihydrobenzo[a]quinolizine-3-carboxylic acid;
(−)-9-[8-(tert-Butoxycarbonylamino)octoxy]-6-tert-butyl-10-methoxy-2-oxo-6,7-dihydrobenzo[a]quinolizine-3-carboxylic acid;
(+)-9-(8-Aminooctoxy)-6-tert-butyl-10-methoxy-2-oxo-6,7-dihydrobenzo[a]quinolizine-3-carboxylic acid hydrochloride;
(−)-9-(8-Aminooctoxy)-6-tert-butyl-10-methoxy-2-oxo-6,7-dihydrobenzo[a]quinolizine-3-carboxylic acid hydrochloride;
9-[5-(tert-Butoxycarbonylamino)pentoxy]-6-tert-butyl-10-methoxy-2-oxo-6,7-dihydrobenzo[a]quinolizine-3-carboxylic acid;
(+)-9-(5-Aminopentoxy)-6-tert-butyl-10-methoxy-2-oxo-6,7-dihydrobenzo[a]quinolizine-3-carboxylic acid hydrochloride;
(−)-9-(5-Aminopentoxy)-6-tert-butyl-10-methoxy-2-oxo-6,7-dihydrobenzo[a]quinolizine-3-carboxylic acid hydrochloride;
9-(5-Acetamidopentoxy)-6-tert-butyl-10-methoxy-2-oxo-6,7-dihydrobenzo[a]quinolizine-3-carboxylic acid;
6-tert-Butyl-9-[5-(methanesulfonamido)pentoxy]-10-methoxy-2-oxo-6,7-dihydrobenzo[a]quinolizine-3-carboxylic acid;
9-(2-Aminoethoxy)-6-tert-butyl-10-methoxy-2-oxo-6,7-dihydrobenzo[a]quinolizine-3-carboxylic acid;
9-[3-(2-Aminoethoxyl)propoxy]-6-tert-butyl-10-methoxy-2-oxo-6,7-dihydrobenzo[a]quinolizine-3-carboxylic acid;
6-tert-Butyl-9-[3-[2-(ethylamino)ethoxy]propoxy]-10-methoxy-2-oxo-6,7-dihydrobenzo[a]quinolizine-3-carboxylic acid;
6-tert-Butyl-9-(3,3-difluoropropoxy)-10-methoxy-2-oxo-6,7-dihydrobenzo[a]quinolizine-3-carboxylic acid;
6-tert-Butyl-9-(1,1-difluoropropoxy)-10-methoxy-2-oxo-6,7-dihydrobenzo[a]quinolizine-3-carboxylic acid;
6-tert-Butyl-9-(1,1-difluoroallyloxy)-10-methoxy-2-oxo-6,7-dihydrobenzo[a]quinolizine-3-carboxylic acid;
6-tert-Butyl-10-methoxy-9-(3-methylsulfanylpropoxy)-2-oxo-6,7-dihydrobenzo[a]quinolizine-3-carboxylic acid;
(+)-6-tert-Butyl-10-methoxy-9-(3-methylsulfanylpropoxy)-2-oxo-6,7-dihydrobenzo[a]quinolizine-3-carboxylic acid;
(−)-6-tert-Butyl-10-methoxy-9-(3-methylsulfanylpropoxy)-2-oxo-6,7-dihydrobenzo[a]quinolizine-3-carboxylic acid;
6-tert-Butyl-10-methoxy-9-(3-methylsulfonylpropoxy)-2-oxo-6,7-dihydrobenzo[a]quinolizine-3-carboxylic acid;
(+)-6-tert-Butyl-10-methoxy-9-(3-methylsulfonylpropoxy)-2-oxo-6,7-dihydrobenzo[a]quinolizine-3-carboxylic acid;
(−)-6-tert-Butyl-10-methoxy-9-(3-methylsulfonylpropoxy)-2-oxo-6,7-dihydrobenzo[a]quinolizine-3-carboxylic acid;
6-tert-Butyl-10-methoxy-9-(2-morpholinoethoxy)-2-oxo-6,7-dihydrobenzo[a]quinolizine-3-carboxylic acid hydrochloride;
(+)-6-tert-Butyl-10-methoxy-9-(2-morpholinoethoxy)-2-oxo-6,7-dihydrobenzo[a]quinolizine-3-carboxylic acid;
(−)-6-tert-Butyl-10-methoxy-9-(2-morpholinoethoxy)-2-oxo-6,7-dihydrobenzo[a]quinolizine-3-carboxylic acid;
6-tert-Butyl-10-methoxy-9-(3-morpholinopropoxy)-2-oxo-6,7-dihydrobenzo[a]quinolizine-3-carboxylic acid hydrochloride;
(+)-6-tert-Butyl-10-methoxy-9-(3-morpholinopropoxy)-2-oxo-6,7-dihydrobenzo[a]quinolizine-3-carboxylic acid;
(−)-6-tert-Butyl-10-methoxy-9-(3-morpholinopropoxy)-2-oxo-6,7-dihydrobenzo[a]quinolizine-3-carboxylic acid;
6-tert-Butyl-10-methoxy-2-oxo-9-[3-(2-oxopyrrolidin-1-yl)propoxy]-6,7-dihydrobenzo[a]quinolizine-3-carboxylic acid;
6-tert-Butyl-10-methoxy-2-oxo-9-(3-pyrrolidin-1-ylpropoxy)-6,7-dihydrobenzo[a]quinolizine-3-carboxylic acid;
6-Cyclobutyl-10-methoxy-9-(3-methoxypropoxy)-2-oxo-6,7-dihydrobenzo[a]quinolizine-3-carboxylic acid;
9,10-Dimethoxy-2-oxo-6-(2,2,2-trifluoroethyl)-6,7-dihydrobenzo[a]quinolizine-3-carboxylic acid;
10-Methoxy-9-(3-methoxypropoxy)-2-oxo-6-(2,2,2-trifluoroethyl)-6,7-dihydrobenzo[a]quinolizine-3-carboxylic acid;
(+)-10-Methoxy-9-(3-methoxypropoxy)-2-oxo-6-(2,2,2-trifluoroethyl)-6,7-dihydrobenzo[a]quinolizine-3-carboxylic acid;
(−)-10-Methoxy-9-(3-methoxypropoxy)-2-oxo-6-(2,2,2-trifluoroethyl)-6,7-dihydrobenzo[a]quinolizine-3-carboxylic acid;
6-tert-Butyl-10-chloro-9-(3-methoxypropoxy)-2-oxo-6,7-dihydrobenzo[a]quinolizine-3-carboxylic acid;
(+)-6-tert-Butyl-10-chloro-9-(3-methoxypropoxy)-2-oxo-6,7-dihydrobenzo[a]quinolizine-3-carboxylic acid;
(−)-6-tert-Butyl-10-chloro-9-(3-methoxypropoxy)-2-oxo-6,7-dihydrobenzo[a]quinolizine-3-carboxylic acid;
10-Chloro-9-(3-methoxypropoxy)-6-(1-methylcyclopropyl)-2-oxo-6,7-dihydrobenzo[a]quinolizine-3-carboxylic acid;
(+)-10-Chloro-9-(3-methoxypropoxy)-6-(1-methylcyclopropyl)-2-oxo-6,7-dihydrobenzo[a]quinolizine-3-carboxylic acid;
(−)-10-Chloro-9-(3-methoxypropoxy)-6-(1-methylcyclopropyl)-2-oxo-6,7-dihydrobenzo[a]quinolizine-3-carboxylic acid;
10-Methoxy-9-(3-methoxypropoxy)-6-(1-methylcyclopropyl)-2-oxo-6,7-dihydrobenzo[a]quinolizine-3-carboxylic acid;
(+)-10-Methoxy-9-(3-methoxypropoxy)-6-(1-methylcyclopropyl)-2-oxo-6,7-dihydrobenzo[a]quinolizine-3-carboxylic acid;
(−)-10-Methoxy-9-(3-methoxypropoxy)-6-(1-methylcyclopropyl)-2-oxo-6,7-dihydrobenzo[a]quinolizine-3-carboxylic acid;
6-Benzyl-9,10-dimethoxy-2-oxo-6,7-dihydrobenzo[a]quinolizine-3-carboxylic acid;
10-Chloro-6-ethyl-9-(2-methoxyethoxy)-7-methyl-2-oxo-6,7-dihydrobenzo[a]quinolizine-3-carboxylic acid;
(6R*,7S*)-10-Chloro-6-ethyl-9-(2-methoxyethoxy)-7-methyl-2-oxo-6,7-dihydrobenzo[a]quinolizine-3-carboxylic acid;
(6R*,7R*)-10-Chloro-6-ethyl-9-(2-methoxyethoxy)-7-methyl-2-oxo-6,7-dihydrobenzo[a]quinolizine-3-carboxylic acid;
10,11-Difluoro-6-isopropyl-9-(3-methoxypropoxy)-2-oxo-6,7-dihydrobenzo[a]quinolizine-3-carboxylic acid;
(+)-10,11-Difluoro-6-isopropyl-9-(3-methoxypropoxy)-2-oxo-6,7-dihydrobenzo[a]quinolizine-3-carboxylic acid;
(−)-10,11-Difluoro-6-isopropyl-9-(3-methoxypropoxy)-2-oxo-6,7-dihydrobenzo[a]quinolizine-3-carboxylic acid;
6-tert-Butyl-10-methoxy-2-oxo-9-(3-pyrazol-1-ylpropoxy)-6,7-dihydrobenzo[a]quinolizine-3-carboxylic acid;
6-tert-Butyl-10-methoxy-2-oxo-9-[3-(1,2,4-triazol-1-yl)propoxy]-6,7-dihydrobenzo[a]quinolizine-3-carboxylic acid;
6-tert-Butyl-9-(3-carboxypropoxy)-10-methoxy-2-oxo-6,7-dihydrobenzo[a]quinolizine-3-carboxylic acid;
9-Bromo-6-methyl-2-oxo-6,7-dihydrobenzo[a]quinolizine-3-carboxylic acid;
11-Bromo-6-methyl-2-oxo-6,7-dihydrobenzo[a]quinolizine-3-carboxylic acid;
(+)-9-Bromo-6-methyl-2-oxo-6,7-dihydrobenzo[a]quinolizine-3-carboxylic acid;
9-(4-tert-Butoxycarbonylpiperazin-1-yl)-6-methyl-2-oxo-6,7-dihydrobenzo[a]quinolizine-3-carboxylic acid;
9-[Benzyl(methyl)amino]-6-methyl-2-oxo-6,7-dihydrobenzo[a]quinolizine-3-carboxylic acid;
6-Methyl-11-(methylamino)-2-oxo-6,7-dihydrobenzo[a]quinolizine-3-carboxylic acid;
6-Ethyl-10-methoxy-2-oxo-9-propyl-6,7-dihydrobenzo[a]quinolizine-3-carboxylic acid;
10-Methoxy-6-methyl-2-oxo-6,7-dihydrobenzo[a]quinolizine-3-carboxylic acid;
9-Bromo-6-ethyl-10-methoxy-2-oxo-6,7-dihydrobenzo[a]quinolizine-3-carboxylic acid;
9-Cyano-6-ethyl-10-methoxy-2-oxo-6,7-dihydrobenzo[a]quinolizine-3-carboxylic acid;
8-Bromo-11-ethoxy-6-ethyl-2-oxo-6,7-dihydrobenzo[a]quinolizine-3-carboxylic acid;
8-Cyano-11-ethoxy-6-ethyl-2-oxo-6,7-dihydrobenzo[a]quinolizine-3-carboxylic acid;
6-Ethyl-9,10-dimethyl-2-oxo-6,7-dihydrobenzo[a]quinolizine-3-carboxylic acid; and
6-Ethyl-8,9-dimethyl-2-oxo-6,7-dihydrobenzo[a]quinolizine-3-carboxylic acid;
or a pharmaceutically acceptable salt thereof.

51. The method of claim 22, wherein the compound of Formula (II) is selected from:
9-benzyloxy-10-methoxy-6-methyl-2-oxo-6,7-dihydrobenzo[a]quinolizine-3-carboxylic acid;
9,10-Diethoxy-6-ethyl-2-oxo-6,7-dihydrobenzo[a]quinolizine-3-carboxylic acid;
(+)-6-Ethyl-10-methoxy-2-oxo-9-(2,2,2-trifluoroethoxy)-6,7-dihydrobenzo[a]quinolizine-3-carboxylic acid;
9-Butoxy-6-ethyl-10-methoxy-2-oxo-6,7-dihydrobenzo[a]quinolizine-3-carboxylic acid;
6-Ethyl-9-(3-hydroxy-2,2-dimethyl-propoxy)-10-methoxy-2-oxo-6,7-dihydrobenzo[a]quinolizine-3-carboxylic acid;
6-Ethyl-10-methoxy-9-(2-methoxyethoxy)-2-oxo-6,7-dihydrobenzo[a]quinolizine-3-carboxylic acid;
9-(Cyclopropylmethoxy)-6-ethyl-10-methoxy-2-oxo-6,7-dihydrobenzo[a]quinolizine-3-carboxylic acid;
6-Ethyl-9-isobutoxy-10-methoxy-2-oxo-6,7-dihydrobenzo[a]quinolizine-3-carboxylic acid;
9-Ethoxy-6-ethyl-10-methoxy-2-oxo-6,7-dihydrobenzo[a]quinolizine-3-carboxylic acid;
6-Ethyl-10-methoxy-9-(3-methoxypropoxy)-2-oxo-6,7-dihydrobenzo[a]quinolizine-3-carboxylic acid;
9-(2-Ethoxyethoxy)-6-ethyl-10-methoxy-2-oxo-6,7-dihydrobenzo[a]quinolizine-3-carboxylic acid;
6-Ethyl-10-methoxy-2-oxo-9-(tetrahydropyran-4-ylmethoxy)-6,7-dihydrobenzo[a]quinolizine-3-carboxylic acid;
6-Ethyl-9,10-dimethoxy-2-oxo-6,7-dihydrobenzo[a]quinolizine-3-carboxylic acid;
(6R)-(+)-6-Ethyl-9,10-dimethoxy-2-oxo-6,7-dihydrobenzo[a]quinolizine-3-carboxylic acid;
6-Ethyl-10-methoxy-2-oxo-9-propoxy-6,7-dihydrobenzo[a]quinolizine-3-carboxylic acid;
6,10-Diethyl-9-methoxy-2-oxo-6,7-dihydrobenzo[a]quinolizine-3-carboxylic acid;
10-Cyclopropyl-6-ethyl-9-methoxy-2-oxo-6,7-dihydrobenzo[a]quinolizine-3-carboxylic acid;
6-Isopropyl-9,10-dimethoxy-2-oxo-6,7-dihydrobenzo[a]quinolizine-3-carboxylic acid;
6-Isobutyl-9,10-dimethoxy-2-oxo-6,7-dihydrobenzo[a]quinolizine-3-carboxylic acid;
10-Chloro-6-isobutyl-9-(2-methoxyethoxy)-2-oxo-6,7-dihydrobenzo[a]quinolizine-3-carboxylic acid;
10-Chloro-6-isopropyl-9-(2-methoxyethoxy)-2-oxo-6,7-dihydrobenzo[a]quinolizine-3-carboxylic acid;
10-Fluoro-6-isopropyl-9-(2-methoxyethoxy)-2-oxo-6,7-dihydrobenzo[a]quinolizine-3-carboxylic acid;
9-Benzyloxy-6-ethyl-10-methyl-2-oxo-6,7-dihydrobenzo[a]quinolizine-3-carboxylic acid;
10-Chloro-6-isopropyl-9-(3-methoxypropoxy)-2-oxo-6,7-dihydrobenzo[a]quinolizine-3-carboxylic acid;
10-Methoxy-6-isopropyl-9-(3-methoxypropoxy)-2-oxo-6,7-dihydrobenzo[a]quinolizine-3-carboxylic acid;
(+)-10-Methoxy-6-isopropyl-9-(3-methoxypropoxy)-2-oxo-6,7-dihydrobenzo[a]quinolizine-3-carboxylic acid;
6-Isopropyl-10-methoxy-2-oxo-9-(2,2,2-trifluoroethoxy)-6,7-dihydrobenzo[a]quinolizine-3-carboxylic acid;
(+)-6-Isopropyl-10-methoxy-9-(2-methoxyethoxy)-2-oxo-6,7-dihydrobenzo[a]quinolizine-3-carboxylic acid;
6-tert-Butyl-10-methoxy-9-(3-methoxypropoxy)-2-oxo-6,7-dihydrobenzo[a]quinolizine-3-carboxylic acid;
(+)-6-tert-Butyl-10-methoxy-9-(3-methoxypropoxy)-2-oxo-6,7-dihydrobenzo[a]quinolizine-3-carboxylic acid;
10-Methoxy-9-(2-methoxyethoxy)-6-(1-methylcyclopropyl)-2-oxo-6,7-dihydrobenzo[a]quinolizine-3-carboxylic acid;
(+)-10-Methoxy-9-(2-methoxyethoxy)-6-(1-methylcyclopropyl)-2-oxo-6,7-dihydrobenzo[a]quinolizine-3-carboxylic acid;
11-Chloro-10-fluoro-6-isopropyl-9-(3-methoxypropoxy)-2-oxo-6,7-dihydrobenzo[a]quinolizine-3-carboxylic acid;
10-Fluoro-6-isopropyl-9-(3-methoxypropoxy)-2-oxo-6,7-dihydrobenzo[a]quinolizine-3-carboxylic acid;
6-tert-Butyl-9-(2,2-difluoro-3-hydroxy-propoxy)-10-methoxy-2-oxo-6,7-dihydrobenzo[a]quinolizine-3-carboxylic acid;
(+)-9-(2,2-Difluoroethoxy)-6-isopropyl-10-methoxy-2-oxo-6,7-dihydrobenzo[a]quinolizine-3-carboxylic acid;
9-(3-Hydroxy-2,2-dimethyl-propoxy)-6-isopropyl-10-methoxy-2-oxo-6,7-dihydrobenzo[a]quinolizine-3-carboxylic acid;
9-(3-Hydroxypropoxy)-6-isopropyl-10-methoxy-2-oxo-6,7-dihydrobenzo[a]quinolizine-3-carboxylic acid;
6-Isopropyl-10-methoxy-9-(4-methoxybutoxy)-2-oxo-6,7-dihydrobenzo[a]quinolizine-3-carboxylic acid;
6-tert-Butyl-9-(3-hydroxy-2,2-dimethyl-propoxy)-10-methoxy-2-oxo-6,7-dihydrobenzo[a]quinolizine-3-carboxylic acid;
6-tert-Butyl-9-(5-hydroxypentoxy)-10-methoxy-2-oxo-6,7-dihydrobenzo[a]quinolizine-3-carboxylic acid;
6-tert-Butyl-9-(6-hydroxyhexoxy)-10-methoxy-2-oxo-6,7-dihydrobenzo[a]quinolizine-3-carboxylic acid;
6-tert-Butyl-9-(4-hydroxybutoxy)-10-methoxy-2-oxo-6,7-dihydrobenzo[a]quinolizine-3-carboxylic acid;
9-(6-Aminohexoxy)-6-tert-butyl-10-methoxy-2-oxo-6,7-dihydrobenzo[a]quinolizine-3-carboxylic acid;
9-[8-(tert-Butoxycarbonylamino) octoxy]-6-tert-butyl-10-methoxy-2-oxo-6,7-dihydrobenzo[a]quinolizine-3-carboxylic acid;
9-[5-(tert-Butoxycarbonylamino)pentoxy]-6-tert-butyl-10-methoxy-2-oxo-6,7-dihydrobenzo[a]quinolizine-3-carboxylic acid;
9-(5-Acetamidopentoxy)-6-tert-butyl-10-methoxy-2-oxo-6,7-dihydrobenzo[a]quinolizine-3-carboxylic acid;
6-tert-Butyl-9-[5-(methanesulfonamido)pentoxy]-10-methoxy-2-oxo-6,7-dihydrobenzo[a]quinolizine-3-carboxylic acid;
6-tert-Butyl-9-[3-[2-(ethylamino)ethoxy]propoxy]-10-methoxy-2-oxo-6,7-dihydrobenzo[a]quinolizine-3-carboxylic acid;
6-tert-Butyl-9-(3,3-difluoropropoxy)-10-methoxy-2-oxo-6,7-dihydrobenzo[a]quinolizine-3-carboxylic acid;
6-tert-Butyl-9-(1,1-difluoropropoxy)-10-methoxy-2-oxo-6,7-dihydrobenzo[a]quinolizine-3-carboxylic acid;
6-tert-Butyl-10-methoxy-9-(3-methylsulfanylpropoxy)-2-oxo-6,7-dihydrobenzo[a]quinolizine-3-carboxylic acid;
6-tert-Butyl-10-methoxy-9-(3-methylsulfonylpropoxy)-2-oxo-6,7-dihydrobenzo[a]quinolizine-3-carboxylic acid;
6-tert-Butyl-10-methoxy-9-(2-morpholinoethoxy)-2-oxo-6,7-dihydrobenzo[a]quinolizine-3-carboxylic acid hydrochloride;
6-tert-Butyl-10-methoxy-9-(3-morpholinopropoxy)-2-oxo-6,7-dihydrobenzo[a]quinolizine-3-carboxylic acid hydrochloride;
6-Cyclobutyl-10-methoxy-9-(3-methoxypropoxy)-2-oxo-6,7-dihydrobenzo[a]quinolizine-3-carboxylic acid;
10-Methoxy-9-(3-methoxypropoxy)-2-oxo-6-(2,2,2-trifluoroethyl)-6,7-dihydrobenzo[a]quinolizine-3-carboxylic acid;
6-tert-Butyl-10-chloro-9-(3-methoxypropoxy)-2-oxo-6,7-dihydrobenzo[a]quinolizine-3-carboxylic acid;
10-Chloro-9-(3-methoxypropoxy)-6-(1-methylcyclopropyl)-2-oxo-6,7-dihydrobenzo[a]quinolizine-3-carboxylic acid;
10-Methoxy-9-(3-methoxypropoxy)-6-(1-methylcyclopropyl)-2-oxo-6,7-dihydrobenzo[a]quinolizine-3-carboxylic acid;
(6R*,7S*)-10-Chloro-6-ethyl-9-(2-methoxyethoxy)-7-methyl-2-6,7-dihydrobenzo[a]quinolizine-3-carboxylic acid; and
10,11-Difluoro-6-isopropyl-9-(3-methoxypropoxy)-2-oxo-6,7-dihydrobenzo[a]quinolizine-3-carboxylic acid,
or a pharmaceutically acceptable salt thereof.

52. The method of claim 22, wherein the compound of Formula (II) is: 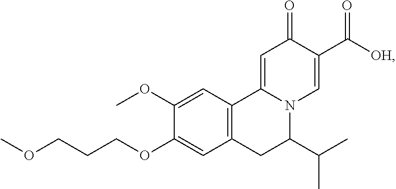 [SMILES: COCCCOc1cc2c(cc1OC)-c1cc(=O)c(C(=O)O)cn1C(C(C)C)C2]
or a pharmaceutically acceptable salt thereof.

53. The method of claim 52, wherein the compound of Formula (II) is: 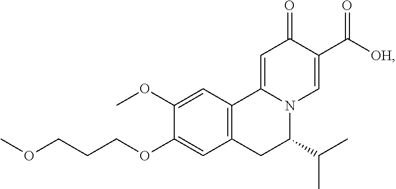 [SMILES: COCCCOc1cc2c(cc1OC)-c1cc(=O)c(C(=O)O)cn1[C@H](C(C)C)C2]
or a pharmaceutically acceptable salt thereof.

54. The method of claim 52, wherein the compound of Formula I is: 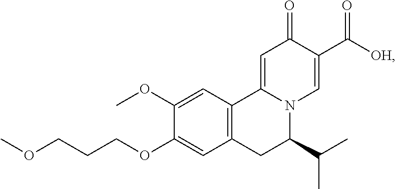 [SMILES: COCCCOc1cc2c(cc1OC)-c1cc(=O)c(C(=O)O)cn1[C@@H](C(C)C)C2]
or a pharmaceutically acceptable salt thereof.

55. The method of claim 22, wherein the compound of Formula (II) is selected from: 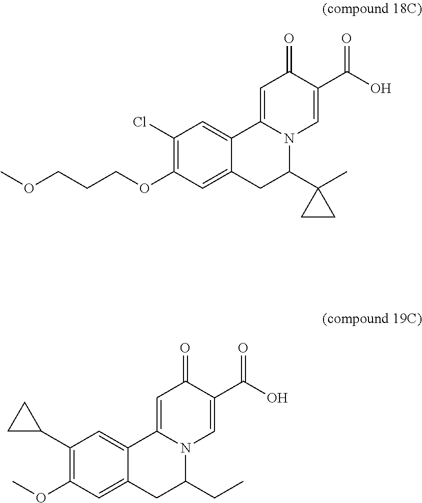 [SMILES: COCCCOc1cc2c(cc1Cl)-c1cc(=O)c(C(=O)O)cn1C(C1(C)CC1)C2 ; CCC1Cc2cc(OC)c(C3CC3)cc2-c2cc(=O)c(C(=O)O)cn21]
or a pharmaceutically acceptable salt thereof.

56. The method of any one of claims 1-55, wherein the disorder associated with telomere or telomerase dysfunction is dyskeratosis congenita, aplastic anemia, pulmonary fibrosis, myelodysplastic syndrome, idiopathic pulmonary fibrosis, hematological disorder, or hepatic fibrosis.

57. The method of any one of claims 1-55, wherein the disorder associated with aging is macular degeneration, diabetes mellitus, osteoarthritis, rheumatoid arthritis, sarcopenia, cardiovascular disease, hypertension, atherosclerosis, coronary artery disease, ischemia/reperfusion injury, cancer, premature death, age-related decline in cognitive function, cardiopulmonary function, muscle strength, vision, or hearing.

58. The method of any one of claims 1-55, wherein the neurodevelopmental disorder is genetic.

59. The method of any one of claim 1-55 or 58, wherein the neurodevelopmental disorder is pontocerebellar hypoplasia.

60. A method of modulating ex vivo expansion of stem cells, the method comprising contacting the cells with an effective amount of a compound as recited in any one of claims 1-55, or a pharmaceutically acceptable salt thereof.

61. A method of modulating non-coding RNAs in a cell, the method comprising contacting the cell with an effective amount of a compound as recited in any one of claims 1-55, or a pharmaceutically acceptable salt thereof.

62. A method of expanding a cell, the method comprising culturing the cell in the presence of an effective amount of a compound as recited in any one of claims 1-55.

63. The method of claim 62, wherein the cell is selected from the group consisting of: stem cell, pluripotent stem cell, hemotopoietic stem cell, and embryonic stem cell.

64. The method of claim 63, wherein the cell is a pluripotent stem cell.

65. The method of claim 63, wherein the cell is a hemotopoietic stem cell.

66. The method of claim 63, wherein the cell is an embryonic stem cell.

67. The method of any of claims 62-66, wherein the cell is collected from a subject with a disease or condition selected from the group consisting of a disorder associated with telomere or telomerase dysfunction, a disorder associated with aging, a pre-leukemic or pre-cancerous condition, and a neurodevelopment disorder.

68. The method of any of claims 62-67, further comprising culturing the cell with a feeder layer in a medium.

69. The method of any one of claims 62-68, wherein the cell has at least one stem cell marker selected from the group consisting of FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, S SEA-4, and Sox-2.

70. The method of claim 69, wherein the stem cell marker is CD34.

71. The method of claim 70, further comprising enriching stem cells by isolating CD34+ cells.

72. The method of claim 67, wherein the subject is a mammal.

73. The method of claim 72, wherein the subject is a human.

74. The method of any one of claims 62-73, comprising culturing the cell in a medium selected from the group consisting of Iscove's modified Dulbecco's Media (IMDM) medium, Dulbecco's Modified Eagle Medium (DMEM), Roswell Park Memorial Institute (RPMI) medium, minimum essential medium alpha medium (α-MEM), Basal Media Eagle (BME) medium, Glasgow Minimum Essential Medium (GMEM), Modified Eagle Medium (MEM), Opti-MEM I Reduced Serum medium, neuroplasma medium, CO₂-Independent medium, and Leibovitz's L-15 medium.

75. The method of claim 62, wherein the cell is a Chimeric Antigen Receptor (CAR) T-Cell.

76. The method of claim 62, wherein the cell is a lymphocyte.

77. The method of claim 62, wherein the cell is a T cell, an engineered T cell, or a natural killer cell (NK).


SEQUENCE LISTING (1 sequence)

SEQ ID NO: 1 - protein, 631 aa, Homo sapiens
MYRSGERLLGSHALPAEQRDFLPLETTNNNNNHHQPGAWARRAGSSASSPPSASSSPHPSAAVPAADPADSASGSSNKRKRDNKASTYGLNYSLLQPSGGRAAGGGRADGGGVVYSGTPWKRRNYNQGVVGLHEEISDFYEYMSPRPEEEKMRMEVVNRIESVIKELWPSADVQIFGSFKTGLYLPTSDIDLVVFGKWENLPLWTLEEALRKHKVADEDSVKVLDKATVPIIKLTDSFTEVKVDISFNVQNGVRAADLIKDFTKKYPVLPYLVLVLKQFLLQRDLNEVFTGGIGSYSLFLMAVSFLQLHPREDACIPNTNYGVLLIEFFELYGRHFNYLKTGIRIKDGGSYVAKDEVQKNMLDGYRPSMLYIEDPLQPGNDVGRSSYGAMQVKQAFDYAYVVLSHAVSPIAKYYPNNETESILGRIIRVTDEVATYRDWISKQWGLKNRPEPSCNGNGVTLIVDTQQLDKCNNNLSEENEALGKCRSKTSESLSKHSSNSSSGPVSSSSATQSSSSDVDSDATPCKTPKQLLCRPSTGNRVGSQDVSLESSQAVGKMQSTQTTNTSNSTNKSQHGSARLFRSSSKGFQGTTQTSHGSLMTNKQHQGKSNNQYYHGKKRKHKRDAPLSDLCR